(19) United States
(12) Patent Application Publication
(10) Pub. No.: US 2018/0147257 A1
(43) Pub. Date: May 31, 2018

(54) BTN3A ECTODOMAIN PROTEINS AND METHODS OF USE

(71) Applicant: The Board of Trustees of the Leland Stanford Junior University, Stanford, CA (US)

(72) Inventors: Daniel Mark Corey, Palo Alto, CA (US); Aaron Michael Ring, New Haven, CT (US); Irving L. Weissman, Stanford, CA (US)

(21) Appl. No.: 15/575,927

(22) PCT Filed: May 20, 2016
(86) PCT No.: PCT/US16/33619

§ 371 Date: Nov. 21, 2017

Related U.S. Application Data
(60) Provisional application No. 62/165,827, filed on May 22, 2015.
(60) Provisional application No. 62/206,454, filed on Aug. 18, 2015.

(51) Int. Cl. A61K 38/17; A61K 39/00; C07K 14/705; A61P 35/00; G01N 33/68

(52) U.S. Cl. CPC A61K 38/177; A61K 39/0011; C07K 14/705; A61K 2039/5158; A61K 2039/5154; G01N 33/68; A61P 35/00

(57) ABSTRACT

BTN3A ectodomain polypeptides are provided, which comprise a BTN3A ectodomain (e.g., a BTN3A1, BTN3A2, or BTN3A3 ectodomain) and lack a BTN3A transmembrane domain (e.g., a BTN3A1, BTN3A2, or BTN3A3 transmembrane domain). Compositions and methods are provided for activating an antigen presenting cell (APC). In some cases, the APC is activated in vivo. For example, in some cases, APC activity is stimulated (an APC is activated) in a mammal by administering a pharmaceutical composition comprising a BTN3A ectodomain polypeptide.

### CROSS-REFERENCE

This application claims the benefit of U.S. Provisional Patent Application Nos. 62/165,827 filed May 22, 2015, and 62/206,454 filed Aug. 18, 2015, which applications are incorporated herein by reference in their entirety.

### GOVERNMENT SUPPORT

This invention was made with Government support under contract CA086065 awarded by the National Institutes of Health. The Government has certain rights in the invention.

### INCORPORATION BY REFERENCE OF SEQUENCE LISTING PROVIDED AS A TEXT FILE

A Sequence Listing is provided herewith as a text file, “STAN-1168WO_SeqList_ST25.txt” created on Mar. 22, 2016 and having a size of 54 KB. The contents of the text file are incorporated by reference herein in their entirety.

### INTRODUCTION

The current model for T-cell activation postulates that naive T-cells require two signals for full activation: (i) a signal provided through the binding of processed antigens presented to the T-cell receptor by major histocompatibility complex (MHC) class I molecules (e.g., via an antigen producing cell (APC)); and (ii) an additional signal provided by the interaction of co-stimulatory molecules on the surface of T-cells and their ligands on antigen presenting cells (APCs). Recognition of an antigen by a naive T-cell is insufficient in itself to trigger T-cell activation. Without a co-stimulatory signal, T-cells may be eliminated either by death or by induction of anergy.

The activation/stimulation of APCs is critical for an appropriate immune response. There is a need in the art for compositions and methods that can activate (e.g., increase the activation of) antigen presenting cells.

The present disclosure provides BTN3A ectodomain polypeptides that activate antigen presenting cells (APCs), which can then stimulate (e.g., cross-prime) immune cells such as T cells (e.g., naive T cells) and thereby enhance an immune response. Methods of use are also provided.

### SUMMARY

Methods and compositions are provided for stimulating the activity of antigen presenting cells (APCs) (stimulating APC activity) in an individual (e.g., in a mammal). In some cases, the methods include a step of administering a composition that includes a BTN3A ectodomain polypeptide (e.g., administering a therapeutic dose of a pharmaceutical composition comprising a BTN3A ectodomain polypeptide). BTN3A ectodomain polypeptides and compositions that include a BTN3A ectodomain polypeptide are also provided. The polypeptides include a BTN3A ectodomain and do not include a BTN3A transmembrane domain. Subject BTN3A ectodomain polypeptides have utility for in vivo and in vitro methods that stimulate antigen presenting cell (APC) activity (e.g., by promoting differentiation of Dendritic Cells (DCs), by activating APCs, etc.).

In the subject methods and compositions, a BTN3A ectodomain polypeptide can be post-translationally modified, for example by glycosylation, PEGylation, etc. A BTN3A ectodomain polypeptide can be a fusion protein (i.e., can include an amino acid sequence in addition to a BTN3A ectodomain), for example a fusion with antibody Fc sequences and/or binding polypeptide (e.g., an antigen binding region of a polypeptide, an ectodomain from a protein other than BTN3A) that provides for specific binding to a target molecule of interest (e.g., an antigen of interest); and the like. BTN3A ectodomain polypeptides can be monomeric or multimeric, i.e. dimer, trimer, tetramer, etc. For example, in some cases, a BTN3A ectodomain polypeptide includes a dimerization moiety. In some cases, a BTN3A ectodomain polypeptide is multispecific, and thus includes a region (in addition to the BTN3A ectodomain) that specifically binds to a target molecule other than BTN3A.

The disclosure also includes pharmaceutical formulations having a BTN3A ectodomain polypeptide in combination with a pharmaceutically acceptable excipient (a pharmaceutical excipient). Such formulations may be provided as a unit dose (a unit dose formulation), e.g. a dose effective to stimulate APC activity. Pharmaceutical formulations also include lyophilized or other preparations of the BTN3A ectodomain polypeptides, which may be reconstituted for use.

In some embodiments, BTN3A ectodomain polypeptides can be administered in combination (co-administered) with another agent, e.g., an opsonizing agent (e.g., an ADCC-inducing antibody) that selectively binds to the targeted cell. For example, in some cases, methods are provided to stimulate an immune response, e.g. by targeting the destruction of living cancer cells by the immune system. In such methods, APC activity is stimulated (e.g., by the administration of a subject BTN3A ectodomain polypeptide), and specific cells are targeted by means of a binding agent (e.g., an antibody, an ectodomain of protein other than BTN3A, etc.) that specifically binds to target cells.

Inflicted individuals that can be treated with a BTN3A ectodomain polypeptide include individuals that have cancer, individuals that harbor an infection (e.g., a chronic infection, a viral infection, etc.), individuals that have an immunological disorder (e.g., a disorder associated with immunosuppression, e.g., primary or combined immunodeficiency), individuals that have an inflammatory disorder, and/or individuals that have other hyper-proliferative conditions, for example sclerosis, fibrosis, and the like, etc. In some cases, cancer cells, e.g. tumor cells, are targeted for elimination by contacting the cells of the immune system with a dose of a BTN3A ectodomain polypeptide that is effective to stimulate APC activity (activate APCs), allowing for increased stimulation of the immune system.

Administration of an effective dose of a BTN3A ectodomain polypeptide to a patient stimulates APC activity (activates APCs) which can increase the clearance of tumor cells and/or infected cells (e.g., chronically infected cells). In some cases, the BTN3A ectodomain polypeptide can be combined (co-administered) with ADCC-inducing antibodies (e.g., opsonizing antibodies, monoclonal antibodies) directed against one or more tumor cell markers, which combination therapy can be synergistic in enhancing elimination of cancer cells as compared to the administration of either agent as a single entity. In other embodiments the BTN3A ectodomain polypeptide comprises a detectable label. Such a labeled reagent can be used, for example, for imaging purposes in vitro or in vivo, e.g. in the imaging of a tumor, in the imaging of APC/T cell interactions, etc. In some cases, a BTN3A ectodomain polypeptide can be used as a diagnostic tool for the detection of cells expressing a receptor (e.g., a counter receptor such as, e.g., LTβR, FLT1, HLA-E, CD163, and/or ROR2) for BTN3A (e.g., BTN3A1, BTN3A2, and/or BTN3A3), and can be used as a companion diagnostic to assess whether a particular treatment regimen has been successful.

Provided are methods for activating an antigen presenting cell (APC). In some embodiments, a subject method includes contacting an APC or a monocyte with a BTN3A ectodomain polypeptide that comprises a BTN3A ectodomain (e.g., a BTN3A1, BTN3A2, or BTN3A3 ectodomain) and lacks a BTN3A transmembrane domain (e.g., a BTN3A1, BTN3A2, or BTN3A3 transmembrane domain), in an amount and for a period of time effect to activate the APC or to induce the monocyte to differentiate and mature into an activated APC. In some cases, the BTN3A ectodomain polypeptide includes a dimerization moiety. In some cases, the BTN3A ectodomain polypeptide is a monomer.

In some cases, the BTN3A ectodomain polypeptide includes a BTN3A ectodomain and a fusion partner. In some cases, the fusion partner is part or whole of an Fc region. In some cases, the Fc region is a human IgG4 Fc region. In some cases, the BTN3A ectodomain polypeptide is a multispecific protein and the fusion partner includes a region that specifically binds to a target molecule that is different from the target molecule bound by the BTN3A ectodomain. In some cases, the BTN3A ectodomain polypeptide is a multimeric protein and the fusion partner includes a region that specifically binds to a target molecule that is different from the target molecule bound by the BTN3A ectodomain. In some cases, the target molecule bound by the BTN3A ectodomain is selected from: LTβR, FLT1, HLA-E, CD163, and ROR2. In some cases, the target molecule bound by the BTN3A ectodomain is LTβR. In some cases, the target molecule bound by the BTN3A ectodomain is FLT1. In some cases, the target molecule bound by the BTN3A ectodomain is HLA-E. In some cases, the target molecule bound by the BTN3A ectodomain is CD163. In some cases, the target molecule bound by the BTN3A ectodomain is ROR2. In some cases, the fusion partner includes a region that specifically binds an antigen selected from: CTLA-4, Lag-3, BTLA, Tim-3, CD244, CD40, CD40L, CD47, SIRPα, PD-1, and PD-L1. In some cases, the BTN3A ectodomain polypeptide includes a detectable label.

In some cases, the contacting includes administering the BTN3A ectodomain polypeptide to an individual with cancer and/or an infectious disease. In some cases, the BTN3A ectodomain polypeptide is co-administered with an ADCC-inducing antibody (e.g., an opsonizing antibody, an ADCC-inducing antibody specifically binds to a tumor antigen, e.g., selected from: CD20, CD52, CD38, HER-2, 17-1A, and EGFR). In some cases, the step of contacting is performed in vitro or ex vivo. In some cases, the method includes contacting the APC or monocyte with a tumor antigen (e.g., a tumor antigen present in a tumor lysate). In some cases, the APC or monocyte is contacted with the tumor antigen and/or the tumor lysate in the presence of the BTN3A ectodomain polypeptide. In some cases, the APC or monocyte is contacted with the tumor antigen and/or tumor lysate prior to or after said contacting with the BTN3A ectodomain polypeptide.

In some cases, the activated APC is introduced into an individual with cancer and/or an infectious disease. In some cases, the activated APC is autologous to the individual. In some cases, the activated APC is used to cross-prime a naive T cell into an antigen specific effector cell. In some cases, the activated APC is contacted in vitro or ex vivo with the naive T cell. In some cases, the antigen specific effector cell is introduced into an individual with cancer and/or an infectious disease. In some cases, the naive T cell is autologous to the individual.

In some cases, the BTN3A ectodomain of a BTN3A ectodomain polypeptide comprises an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in any of SEQ ID NOs: 10, 13, and 15. In some cases, a BTN3A ectodomain polypeptide comprises a BTN3A ectodomain and a fusion partner, where the fusion partner is a dimerization moiety. In some cases, the dimerization moiety comprises an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in any of SEQ ID NOs: 31-34.

Also provided are pharmaceutical compositions (e.g., BTN3A ectodomain compositions). In some embodiments, a subject BTN3A ectodomain composition includes (a) a BTN3A ectodomain polypeptide comprising a BTN3A ectodomain (e.g., a BTN3A1, BTN3A2, or BTN3A3 ectodomain) and lacking a BTN3A transmembrane domain (e.g., a BTN3A1, BTN3A2, or BTN3A3 transmembrane domain); and (b) a pharmaceutical excipient. In some cases, the composition is a unit dose formulation that is effective to activate antigen presenting cells (APCs) in an individual. In some cases, the BTN3A ectodomain polypeptide includes a dimerization moiety. In some cases, the BTN3A ectodomain polypeptide is a monomer. In some cases, the BTN3A ectodomain polypeptide includes a BTN3A ectodomain and a fusion partner. In some cases, the fusion partner is part or whole of an Fc region (e.g., a human IgG4 Fc region). In some cases, the BTN3A ectodomain polypeptide is a multispecific protein and the fusion partner includes a region that specifically binds to a target molecule that is different from the target molecule bound by the BTN3A ectodomain (e.g., different than LTβR, FLT1, HLA-E, CD163, and/or ROR2). In some cases, the BTN3A ectodomain polypeptide is a multimeric protein and the fusion partner includes a region that specifically binds to a target molecule that is different from the target molecule bound by the BTN3A ectodomain (e.g., different than LTβR, FLT1, HLA-E, CD163, and/or ROR2). In some cases, the fusion partner includes a region that specifically binds to a tumor antigen (e.g., a tumor antigen selected from: CTLA-4, Lag-3, BTLA, Tim-3, CD244, CD40, CD40L, CD47, SIRPα, PD-1, and PD-L1). In some cases, the BTN3A ectodomain polypeptide includes a detectable label. In some cases, that composition further includes an ADCC-inducing antibody (e.g., an opsonizing antibody). In some cases, the ADCC-inducing antibody (e.g., the opsonizing antibody) specifically binds to a tumor antigen (e.g., a tumor antigen selected from: CD20, CD52, CD38, HER-2, 17-1A, and EGFR).

Also provided are methods of treating an individual having cancer and/or having a chronic infection. In some embodiments, such methods include administering to the individual, a pharmaceutical BTN3A ectodomain composition, in an amount effective to reduce the number of cancer cells and/or infected cells in the individual. In some cases, the individual is a human. In some cases, the method includes co-administering the pharmaceutical BTN3A ectodomain composition with an ADCC-inducing antibody (e.g., an opsonizing antibody, an antibody that binds to a tumor antigen, e.g., selected from: CD20, CD52, CD38, HER-2, 17-1A, and EGFR). In some cases, the pharmaceutical BTN3A ectodomain composition and the ADCC-inducing antibody (e.g., an opsonizing antibody) are not administered simultaneously. In some cases, the pharmaceutical BTN3A ectodomain composition and the ADCC-inducing antibody (e.g., an opsonizing antibody) are administered simultaneously.

Also provided are BTN3A ectodomain polypeptides, or nucleic acids encoding said BTN3A ectodomain polypeptides. In some cases, a subject BTN3A ectodomain polypeptide includes a BTN3A ectodomain (e.g., a BTN3A1, BTN3A2, or BTN3A3 ectodomain) and a dimerization moiety, and lacks a BTN3A transmembrane domain (e.g., a BTN3A1, BTN3A2, or BTN3A3 transmembrane domain). In some cases, a subject BTN3A ectodomain polypeptide includes a BTN3A ectodomain (e.g., a BTN3A1, BTN3A2, or BTN3A3 ectodomain) and a human IgG4 Fc region, and lacks a BTN3A transmembrane domain (e.g., a BTN3A1, BTN3A2, or BTN3A3 transmembrane domain).

Also provided are methods of enhancing immune responses to an antigenic compound. In some embodiments, such methods include administering to a host: (a) a BTN3A ectodomain polypeptide comprising a BTN3A ectodomain and lacking a BTN3A transmembrane domain; and (b) an antigen. In some cases, the source of the antigen is selected from: a human, a non-human animal, a plant, a bacterial cell, an archaeal cell, a fungus, a virus, a parasite, and a cancer cell. In some cases, the host is human. In some cases, the host is a non-human animal. In some cases, the antigen is a vaccine. In some cases, the vaccine is directed at Tuberculosis, Malaria, Human Immunodeficiency Virus (HIV), RotaVirus, Herpes Simplex Virus (HSV), or Cytomegalovirus (CMV). In some cases, the vaccine is a cancer vaccine.

Also provided are methods of identifying and/or generating a high affinity BTN3A ectodomain polypeptide (e.g., methods of identifying a high affinity BTN3A ectodomain polypeptide). A method of identifying a high affinity BTN3A ectodomain polypeptide can include: (a) contacting population of cells that includes one or more monocytes or one or more antigen presenting cells (APCs), with a candidate agent to generate an agent contacted cell population, where the candidate agent is a candidate high affinity BTN3A ectodomain polypeptide; (b) measuring one or more parameters for cells of the agent contacted cell population selected from: secretion of one or more helper cytokines, the production of one or more costimulatory molecules, and one or more downstream effector functions; (c) determining that said contacting resulted in one or more of: an increase in secretion of the one or more helper cytokines, an increase in the production of the one or more costimulatory molecules, and enhancement of the one or more downstream effector functions, where the increase and/or enhancement is relative to a control value (e.g., the parameter as observed when contacting a comparable cell population with a BTN3A ectodomain polypeptide that is not a high affinity BTN3A ectodomain polypeptide); and (d) determining that the candidate agent is a high affinity BTN3A ectodomain polypeptide. In some cases step (c) includes determining that the candidate agent resulted in, upon contacting the cell population, two or more of: an increase in secretion of the one or more helper cytokines, an increase in the production of the one or more costimulatory molecules, and enhancement of the one or more downstream effector functions, where the increase and/or enhancement is relative to a control value (e.g., the parameter as observed when contacting a comparable cell population with a BTN3A ectodomain polypeptide that is not a high affinity BTN3A ectodomain polypeptide). In some cases step (c) includes determining that the candidate agent resulted in, upon contacting the cell population, an increase in secretion of the one or more helper cytokines, an increase in the production of the one or more costimulatory molecules, and enhancement of the one or more downstream effector functions, where the increase and/or enhancement is relative to a control value (e.g., the parameter as observed when contacting a comparable cell population with a BTN3A ectodomain polypeptide that is not a high affinity BTN3A ectodomain polypeptide). In some embodiments, a method of identifying a high affinity BTN3A ectodomain polypeptide can include measuring the affinity of a candidate high affinity BTN3A ectodomain polypeptide for a target molecule, comparing the affinity to a control value (e.g., the binding affinity of a corresponding wild type BTN3A ectodomain for the target molecule), determining that the candidate high affinity BTN3A ectodomain polypeptide has an greater affinity than the control value, and determining that the candidate high affinity BTN3A ectodomain polypeptide is a high affinity BTN3A ectodomain polypeptide. In some cases, such methods can include a step of mutating a nucleic acid encoding a BTN3A ectodomain polypeptide to generate a nucleic acid encoding a candidate high affinity BTN3A ectodomain polypeptide.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1C. Generation of recombinant human BTN3A1-Fc fusion protein. (FIG. 1A) The ectodomain (extracellular domain; IgV+IgC) of BTN3A1 (BTN3A1) was cloned by PCR into an Fc link vector to generate BTN3A1-IgG4 fusion protein. Plasmid DNA was transfected into 293F cells, then Fc-protein enriched supernatants were collected for protein A purification. (FIG. 1B) Protein sequence of an example of a BTN3A ectodomain polypeptide that includes a BTNA3 ectodomain and a fusion partner, where the fusion partner is a human IgG4 Fc region (a recombinant human BTN3A1-Fc fusion protein with a signal sequence and an IgG4 Fc fusion domain) (SEQ ID NO: 20) (FIG. 1C) Protein sequence of an example of a BTNA ectodomain polypeptide having a dimerization moiety (SEQ ID NO: 21). Amino acids 39-255 of SEQ ID NO: 21 are an example of a BTNA ectodomain; amino acids 259-295 of SEQ ID NO: 21 are an example of a dimerization moiety; amino acids 1-38 of SEQ ID NO: 21 are an example of a signal sequence; and amino acids 296-303 of SEQ ID NO: 21 are an example of a His tag (an affinity tag).

FIG. 2A-2B. Analysis of BTN3A1-Fc binding to human immune cells. (FIG. 2A) BTN3A1-Fc AlexaFlour 647 bioconjugate was applied to freshly isolated PBMCs to evaluate BTN3A1 counter receptor expression. Broad staining was observed across multiple subsets of peripheral blood. (FIG. 2B) Histogram plots demonstrate exposure to γ interferon, TLR ligands, and virus stimulation upregulate CD277 counter-receptor expression.

FIG. 3A-3B. BTN3A1-Fc enhances CD3/CD28 mediated activation. (FIG. 3A) PBMCs (1×10⁵ cells/well) were cultured in 96-well plates with CD3/CD28 dynabeads and the indicated concentrations of either BTN3A1-Fc or isotype control Ig. After 12 hours of culture samples were stained with the T cell activation marker CD137 (4-1BB) for FACs analysis. BTN3A1-Fc enhanced activation of T cells across all subsets measured. (FIG. 3B) In the absence of CD3 engagement, stimulation through BTN3A1 did not alter T cell activation.

FIG. 4A-4E. BTN3A1 ectodomain polypeptides induced activation and maturation of dendritic cells (DCs) and monocyte populations. (FIG. 4A) Heat map of MFI data from mass cytometry displaying signaling changes 30 minutes after administration of engineered BTN3A1 ectodomain polypeptides (50 ug/ml). (FIG. 4B) BTN3A1 proteins promote differentiation of Dendritic Cells from monocytes. Purified monocytes were plated in standard culture media (RPMI+5% Human Serum) and stimulated with BTN3A1-Fc or Dimer proteins for 72 hours. The percentage of CD14-CD11C+MHCII+ cells were quantified by FACs from the bulk culture. (FIG. 4C) MFI FACs data from purified lineage negative CD14+ monocyte populations upon activation with BTN3A1 ectodomain polypeptides showing upregulation of costimulatory molecules CD80 and CD86. (FIG. 4D) Cell surface profiling of monocytes and dendritic cells 24 hours after administration of BTN3A1—Fc fusion proteins (25 ug/mL). (FIG. 4E) BTN3A1 dimer proteins signal through NF-kappa-B complexes. Purified monocytes were stimulated with BTN3A1 dimer proteins at indicated concentrations and stained for the intracellular inhibitor of dimeric NF-kappa-B (I-kappa-B-alpha). Histogram plots indicates rapid degradation of I-kappa-B-alpha, an NF-kappa-B inhibitor, 30 minutes after stimulation with BTN3A1 proteins.

FIG. 5A-5C. BTN3A1 ectodomain polypeptides promoted expansion of antigen specific T cells. (FIG. 5A) SPADE clustering algorithm applied to CYTOF data showing changes in CD40L expression 24 hours after administration of BTN3A1-Fc protein (arrows=dendritic and monocyte populations) (DCs: Dendritic Cells). Median intensity values of protein expression are denoted by the node color (blue:minimum; red:maximum) (FIG. 5B) Micrographs of Day 5 co-culture of naive CD8+ T cells with monocyte derived dendritic cells matured with BTN3A1 ectodomain polypeptides. Clusters of T cells are in close apposition to APCs in the Dimer and Monomer groups, giving a similar morphology to the IgG activated groups. (FIG. 5C) Quantitative comparison of antigen specific (“Tetramer +”) and CD8+ T cell expansion 10 days after maturing monocyte derived DC's with BTN3A1 ectodomain dimer.

FIG. 6A-6B. BTN3A1 ectodomain polypeptides induced differentiation of monocytes into CD16+ monocytes with enhanced costimulatory properties. BTN3A1-Fc promoted differentiation of CD16+ monocytes that exhibited elevated levels of costimulatory molecules, closely resembling DC's. (FIG. 6A) Histogram plots from FACs analysis of PBMCs 24 hours after administration of BTN3A1-Fc showing CD16+ monocytes from PBMC. (FIG. 6B) Luminex cytokine profiling from overnight stimulated PBMC's.

FIG. 7. BTN3A1 ectodomain polypeptides induced B and T lymphocyte activation. Heat map of MFI data from mass cytometry displaying cell surface and intracellular cytokine changes 24 hours after administration of BTN3A1-Fc fusion proteins (25 ug/mL).

FIG. 8. BTN3A1 ectodomain polypeptides augmented Antibody-Dependent Cell-mediated Cytoxicity (ADCC). These experiments were carried out using a 10:1 ratio of PBMCs to target cells.

FIG. 9A-9B. Stained tissue biopsy specimens from individuals with acute viral infection. (FIG. 9A) Tissue biopsy specimens stained with anti-BTN3A1 (green) and CD8 (red) from an individual presenting with an HSV2 skin lesion. In the acute setting, robust BTN3A1 staining is seen within infiltrating CD8 T cell populations compared to contralateral control biopsies. Over the course of the next 3 weeks, BTN3A1 expression diminishes to nearly absent levels. (FIG. 9B) Biopsy specimens from an acute HSV2 skin lesion stained with anti-BTN3A1 (green) and lymphocyte markers (CD4, CD8 (red), CD20, and CD56).

FIG. 10A-10B. Expansion and cytotoxicity of antigen specific CD8 T cells after monocyte priming with BTN3A1 ectodomain polypeptides. (FIG. 10A) Melan-A-specific T cells were expanded under different conditions. Monocytes were matured in the presence of BTN3A1 ectodomain polypeptides (with no additional cytokines) and pulsed with Melan-A peptide and the resulting T cell responses were evaluated on day 12. In the absence of BTN3A1 ectodomain polypeptides, monocytes primed naïve CD8 T cells poorly. Priming monocytes with the monomeric form of the ectodomain of BTN3A1 (independent of Fc) increased the fraction of degranulated tumor specific T cells and their cytotoxicity. (FIG. 10B) Priming monocytes with BTN3A1 ectodomain polypeptides promoted expansion of CD8 T cells in tissue culture.

FIG. 11. BTN3A1 ectodomain polypeptides enhanced killing of T cell engaged targets. The B cell lymphoma cell line (Raji) was treated with a single chain bi-specific antibody linking the variable domains of CD3 and CD19 (“BiTE”) with and without BTN3A1 ectodomain polypeptides. Targets were set-up in a 5:1 Effector: Target ratio using fresh PBMCs (PBMC:Raji 5:1). BTN3A1 ectodomain polypeptides significantly enhanced killing of lymphoma targets.

FIG. 12. A multiple sequence alignment of example sequences that include a BTN3A1 (SEQ ID NO: 10), BTN3A2 (SEQ ID NO: 13), or BTN3A3 (SEQ ID NO: 15) ectodomain. The depicted sequences are from the extracellular region of the wild type BTN3A1, BTN3A2, and BTN3A3 proteins.

FIG. 13A-13B. Schematic of FACs-based selection screen from a pooled lentiCRISPR-Cas9 knockout library (GeCKO) used to pinpoint gene targets involved in BTN3A binding. (FIG. 13A) Flowchart summary of selection process. (FIG. 13B) Histogram overlays assessing BTN3A1-Fc fluorescent staining of the GeCKO library at each round (Rd) of selection. The right panel shows the normalized distribution of sgRNAs in naïve (bottom) and Rd5 libraries (top).

FIG. 14. sgRNAs across the genome are depicted as a boxplot showing the median (horizontal line), 25th to 75th percentiles (within the box), and 1st to 99th percentiles (whiskers). Top binding receptor candidates are indicated as colored dots. [See example 3].

FIG. 15. Yeast cells displaying the full-length BTN3A1 ectodomain were stained with purified LTβR-IgG1 fusion protein (LTBR-IgG1 fusion protein) and measured by FACs for staining. As a control, the closest CD277 structural homologue, PDL1 was displayed. BTN3A1 induced yeast specifically bound to LtβR-IgG1, while yeast displaying PDL1 did not. The top panel is a graphic form of the quantification of the bottom panel (which shows measurement of detected signal at different concentrations of the LTBR-IgG1 fusion protein).

FIG. 16. Silver stain to detect proteins on polyacrylamide gel from purified recombinant protein pulldowns. To evaluate for specific binding interactions, purified LtβR-IgG1 and recombinant BTN3A1 monomers and dimers were incubated overnight. Protein complexes were captured on Protein A agarose affinity beads, washed in PBS, and eluted in SDS-PAGE sample buffer. CD277 monomer and dimers specifically bound to LtβR-IgG1.

### DETAILED DESCRIPTION

BTN3A ectodomain polypeptides are provided, which comprise a BTN3A ectodomain (e.g., a BTN3A1, BTN3A2, or BTN3A3 ectodomain) and lack a BTN3A transmembrane domain (e.g., a BTN3A1, BTN3A2, or BTN3A3 transmembrane domain). Compositions and methods are provided for activating an antigen presenting cell (APC). In some cases, the APC is activated in vivo. For example, in some cases, APC activity is stimulated (an APC is activated) in a mammal by administering a pharmaceutical composition comprising a BTN3A ectodomain polypeptide.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed

### Definitions

In the description that follows, a number of terms conventionally used in the field are utilized. In order to provide a clear and consistent understanding of the specification and claims, and the scope to be given to such terms, the following definitions are provided.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is human.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias.

As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions. In some cases, the cancer cells express PD-L1. In some cases, the cancer cells do not express PD-L1 (e.g., in such cases, cells of the immune system of the individual being treated express PD-L1).

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratoma is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Leukemias are cancers that start in blood-forming tissue, such as the bone marrow, and causes large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Sternberg cell. There are currently 6 recognized types of HL. Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of disease progression (e.g., cancer-attributable death or progression), including recurrence, metastatic spread of cancer, and drug resistance. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{d}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

As used herein, the phrase “disease-free survival,” refers to the lack of such tumor recurrence and/or spread and the fate of a patient after diagnosis, with respect to the effects of the cancer on the life-span of the patient. The phrase “overall survival” refers to the fate of the patient after diagnosis, despite the possibility that the cause of death in a patient is not directly due to the effects of the cancer. The phrases, “likelihood of disease-free survival”, “risk of recurrence” and variants thereof, refer to the probability of tumor recurrence or spread in a patient subsequent to diagnosis of cancer, wherein the probability is determined according to the process of the disclosure.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use in addition to those for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this disclosure can be present in unsalified or unesterified form, or in salified and/or esterified form, and the naming of such compounds is intended to include both the original (unsalified and unesterified) compound and its pharmaceutically acceptable salts and esters. Also, certain compounds named in this disclosure may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The term “target cell” as used herein refers to a cell targeted for destruction by the immune system after administration of a subject BTN3A ectodomain polypeptide. A target cell need not express a receptor or counter receptor for BTN3A (e.g., a target cell need to bind to a subject BTN3A ectodomain polypeptide). Instead, administration of a subject BTN3A ectodomain polypeptide leads to stimulation of the immune system (via stimulation of APC activity), thereby leading to the destruction of the target cell. In some cases, a target cell expresses a receptor (or counter receptor) for BTN3A. In some cases, the target cell is determined by the interactions of the APC and the naive T-cell (e.g., determined by the antigen(s) that are presented by the APC(s)).

In some cases, a target cell is an “inflicted” cell (e.g., a cell from an “inflicted” individual), where the term “inflicted” is used herein to refer to a subject with symptoms, an illness, or a disease that can be treated with a subject BTN3A ectodomain polypeptide. An “inflicted” individual can have cancer, can harbor an infection (e.g., a chronic infection), can have an immunological disorder (e.g., a disorder associated with immunosuppression), can have an inflammatory disorder, and/or can have other hyper-proliferative conditions, for example sclerosis, fibrosis, and the like, etc. “Inflicted cells” can be those cells that cause the symptoms, illness, or disease. As non-limiting examples, the inflicted cells of an inflicted patient can be cancer cells, infected cells, inflammatory cells, and the like. In some cases, the inflicted cell (e.g., cancer cell) does not express a receptor (or counter receptor) for BTN3A (e.g., LTβR, FLT1, HLA-E, CD163, and/or ROR2), but the disease (e.g., cancer) can still be treated using a subject BTN3A ectodomain polypeptide because the BTN3A ectodomain polypeptide is used to increase APC activity (e.g., stimulate APCs).

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, those with an immune disorder, etc.) as well as those in which prevention is desired (e.g., those with increased susceptibility to cancer, those with an increased likelihood of infection, those suspected of having cancer, those suspected of harboring an infection, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The word “label” when used herein refers to a detectable compound or composition which is conjugated directly or indirectly to a subject BTN3A ectodomain polypeptide. The label may itself be directly detectable (detectable by itself, e.g., radioisotope labels, fluorescent labels, etc.) or can be detected indirectly (e.g., an enzymatic label, which may catalyze chemical alteration of a substrate compound or composition which is detectable).

By “solid phase” is meant a non-aqueous matrix to which a BTN3A ectodomain polypeptide of the present disclosure can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas. “Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv see Pluckthun, in The Pharmacology of Monoclonal Antibodies, vol. 113, Rosenburg and Moore eds., Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, IgA₂. The heavy-chain constant domains that correspond to the different classes of immunoglobulins are called a, d, e, g, and m, respectively. The subunit structures and three-dimensional configurations of different classes of immunoglobulins are well known. Engineered variants of immunoglobulin subclasses, including those that increase or decrease immune effector functions, half-life, or serum-stability, are also encompassed by this terminology.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

Unless specifically indicated to the contrary, the term “conjugate” as described and claimed herein is defined as a heterogeneous molecule formed by the covalent attachment of one or more antibody fragment(s) to one or more polymer molecule(s), wherein the heterogeneous molecule is water soluble, i.e. soluble in physiological fluids such as blood, and wherein the heterogeneous molecule is free of any structured aggregate. A conjugate of interest is PEG. In the context of the foregoing definition, the term “structured aggregate” refers to (1) any aggregate of molecules in aqueous solution having a spheroid or spheroid shell structure, such that the heterogeneous molecule is not in a micelle or other emulsion structure, and is not anchored to a lipid bilayer, vesicle or liposome; and (2) any aggregate of molecules in solid or insolubilized form, such as a chromatography bead matrix, that does not release the heterogeneous molecule into solution upon contact with an aqueous phase. Accordingly, the term “conjugate” as defined herein encompasses the aforementioned heterogeneous molecule in a precipitate, sediment, bioerodible matrix or other solid capable of releasing the heterogeneous molecule into aqueous solution upon hydration of the solid.

As used in this disclosure, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

### Compositions

BTN3A ectodomain polypeptides and analogs thereof are provided, which may be referred to generically as BTN3A ectodomain reagents. The present disclosure provides a BTN3A ectodomain polypeptide, where the polypeptide lacks the BTN3A transmembrane domain (and can be a soluble BTN3A ectodomain polypeptide) and includes a BTN3A ectodomain. A subject BTN3A ectodomain polypeptide stimulates (induces, increases) activity of antigen presenting cells) APCs.

### Polypeptides

An extracellular domain of protein that is normally tethered to the plasma membrane of a cell is sometimes referred to in the art as an ectodomain. A “BTN3A ectodomain” or “extracellular domain of BTN3A” as used herein refers to a polypeptide having the portion of a BTN3A protein that is sufficient to stimulate (e.g., increase) antigen presenting cell (APC) activity (e.g, sufficient to activate APCs), but which lacks a transmembrane domain (e.g., lacks the naturally present transmembrane domain of a wild type BTN3A protein). Thus, unlike a naturally existing BTN3A protein, a BTN3A ectodomain is not permanently tethered to a cell membrane by way of a transmembrane domain. A BTN3A ectodomain can be considered to be (or be derived from) an ectodomain of a wild type BTN3A (e.g., BTN3A1, BTN3A2, BTN3A3), or can be considered to include at least a portion of (or a portion that is derived from) the ectodomain of a wild type BTN3A protein (e.g., BTN3A1, BTN3A2, BTN3A3 protein). For example, a BTN3A ectodomain can be a fragment of the extracellular domain of a wild type BTN3A protein that retains sufficient activity to activate an APC (i.e., stimulate APC activity).

In some cases, a BTN3A ectodomain polypeptide consists of a BTN3A ectodomain. In some cases, a BTN3A ectodomain polypeptide consists essentially of a BTN3A ectodomain. In some cases, a BTN3A ectodomain polypeptide consists of a BTN3A ectodomain fused to a fusion partner. In some cases, a BTN3A ectodomain polypeptide consists essentially of a BTN3A ectodomain fused to a fusion partner.

As used herein, the term “APC” or “antigen presenting cell” refers to a cell that expresses major histocompatibility complex class II (MHC class II) proteins on its cell membrane surface and is capable of presenting antigens in complex with MHC class II to T-cells, thereby activating T-cells to the presented antigens. The term APC as used herein encompasses dendritic cells, macrophages, and B cells. Monocytes are precursor cells that can differentiate (and mature) into APCs (e.g., activated APCs), and can be induced/stimulated to do so. In some cases, an APC of the subject methods and/or compositions is a dendritic cell. In some cases, an APC of the subject methods and/or compositions is a macrophage. In some cases, an APC of the subject methods and/or compositions is a B-cell. In some cases, an APC of the subject methods and/or compositions is a dendritic cell, macrophage, or B-cell. In some cases, an APC of the subject methods and/or compositions is a dendritic cell or a macrophage. In some cases, an APC of the subject methods and/or compositions is a dendritic cell or a B-cell.

In some cases, an APC of the subject methods and/or compositions is a not a B-cell. In some cases, an APC of the subject methods and/or compositions is not a macrophage. In some cases, an APC of the subject methods and/or compositions is not a B-cell or a macrophage. In some cases, an APC of the subject methods and/or compositions is not a dendritic cell.

Dendritic Cells.

A dendritic cell (DC) is a type of antigen-presenting cell of the mammalian immune system. The term “dendritic cell” as used herein refers to any member of a diverse population of morphologically similar cell types found in lymphoid or non-lymphoid tissues. These cells are characterized by their distinctive morphology and high levels of surface MHC-class II expression (Steinman, et al., Ann. Rev. Immunol. 9:271 (1991); hereby incorporated by reference for its description of such cells).

Dendritic cells are present in nearly all tissues such as the skin and the inner lining of the nose, lungs, liver, stomach, and intestines, as well as in bone marrow, blood, spleen, and lymph nodes. Once activated, DC migrate to the lymph nodes where they interact with T cells and B cells to initiate and shape the adaptive immune response. At certain development stages DC grow branched projections (the dendrites) that give the cells their name. Examples of dendritic cells include bone marrow-derived dendritic cells (BMDC), plasmacytoid dendritic cells, Langerhans cells, interdigitating cells, veiled cells, and dermal dendritic cells. In some cases, a DC expresses at least one marker selected from: CD11 (e.g., CD11a and/or CD11c), MHC-class II (for example, in the case of human, HLA-DR, HLA-DP and HLA-DQ), CD40, CD80 and CD86. In some cases, a DC is positive for HLA-DR and CD83, and negative for CD14. In general DC can be identified (e.g., the presence of DC can be verified) based on any or all of the markers: CD11c+; CD14−/low; CD80+; CD86++; MHC Class I++, MHC Class II+++; CD40++; CD83+/−; CCR7+/−. In some cases, the DC is CD11b+/Gr1^{neg}/CD11c⁺/MHCII⁺/CD64^{dull}. In some cases, the DC is CD11b^{neg}/CD11c^{hi}/MHCII⁺.

In some cases, the dendritic cell expresses a specific Ig Fc receptor. For example, a dendritic cell can express an Fc-γ receptor which recognizes IgG antibodies, or antibodies that contain an Fc region of an IgG. As another example, the dendritic cell can express an Fc-α receptor which recognizes IgA antibodies, or antibodies that contain an Fc region of an IgA. As yet another example, the dendritic cell can express an Fc-ε receptor which recognizes IgE antibodies, or antibodies that contain an Fc region of an IgE. In some cases, dendritic cells expressing a specific Fc receptor are obtained and loaded with an appropriate bridging molecule (e.g., allogeneic Ig of a class recognized by the dendritic cell Fc receptor).

In some embodiments, subject methods include a step of obtaining or isolating a DC (e.g., isolating enriched populations of DC). Techniques for the isolation, generation, and culture of DC will be known to one of ordinary skill in the art and any convenient technique can be used. In some cases, the DC are autologous to the individual who is being treated (i.e., are cells isolated from the individual or are cells derived from cells of the individual).

Macrophages.

A macrophage is a type of antigen-presenting cell of the mammalian immune system. The term “macrophage” as used herein refers to any member of a diverse population of morphologically similar cell types found in lymphoid or non-lymphoid tissues. These cells are characterized by their distinctive morphology and high levels of surface MHC-class II expression. A macrophage is a monocyte-derived phagocyte which is not a dendritic cell or a cell that derives from tissue macrophages by local proliferation. In the body these cells are tissue specific and refer to e. g. Kupffer cells in the liver, alveolar macrophages in the lung, microglia cells in the brain, osteoclasts in the bone etc. The skilled person is aware how to identify macrophage cells, how to isolate macrophage cells from the body of a human or animal, and how to characterize macrophage cells with respect to their subclass and subpopulation (Kruisbeek, 2001; Davies and Gordon 2005 a and b; Zhang et al., 2008; Mosser and Zhang, 2008; Weischenfeldt and Porse, 2008; Ray and Dittel, 2010; Martinez et al., 2008; Jenkins et al., 2011).

Macrophages can be activated by different mechanisms into different subclasses, including, but not limited to M1, M2, M2a, M2b, and M2c subclasses. Whereas the term M1 is used to describe classically activated macrophages that arise due to injury or bacterial infection and IFN-y activation, M2 is a generic term for numerous forms of macrophages activated differently than M1. The M2 classification has further been divided into subpopulations (Mantovani et al., 2004). The most representative form is M2a macrophages, which commonly occur in helminth infections by exposure to worm induced Th2 cytokines IL-4 and IL-13. M2a macrophages were, among others, shown to be essentially involved in protecting the host from re-infection (Anthony et al., 2006) or in contributing to wound healing and tissue remodeling (Gordon, 2003). Another subpopulation is M2b macrophages that produce high levels of IL-10 and low levels of IL-12 but are not per se anti-inflammatory (Anderson and Mosser, 2002; Edwards et al., 2006). M2b macrophages are elicited by immune complexes that bind to Fc-γ receptors in combination with TLR ligands. Finally, M2c macrophages represent a subtype elicited by IL-10, TGF-β or glucocorticoids (Martinez et al., 2008).

Thus, “M2a macrophages” refers to a macrophage cell that has been exposed to a milieu under Th2 conditions (e g. exposure to Th2 cytokines IL-4 and IL-13) and exhibits a specific phenotype by higher expression of the gene Ym1 and/or the gene CD206 and/or the gene RELM-α and/or the gene Arginase-1. Similarly, “M2b macrophages” refers to a macrophage cell that has been exposed to a milieu of immune complexes in combination with TLR or TNF-alpha stimulation. Said cell is characterized through higher expression of the gene SPHK-1 and/or the gene LIGHT and/or the gene IL-10.

In some cases, the present application refers to a macrophage cell “derived from the body of a patient”. This is meant to designate that either macrophages are obtained from the body of said patient, or macrophage precursor cells are obtained from the body of said patient and subsequently differentiated into macrophage cells in vitro as described in Wahl et al. 2006; Davis and Gordon 2005; Smythies et al., 2006; Zhang et al., 2008; Mosser and Zhang, 2008.

B-cells.

A B-cell is a type of antigen-presenting cell of the mammalian immune system. The term “B-cell” as used herein refers to B-cells from any stage of development (e.g., B-stem cells, progenitor B-cells, differentiated B-cells, plasma cells) and from any source including, but not limited to peripheral blood, a region at, in, or near a tumor, lymph nodes, bone marrow, umbilical chord blood, or spleen cells.

B-cell precursors reside in the bone marrow where immature B-cells are produced. B-cell development occurs through several stages, each stage representing a change in the genome content at the antibody loci. In the genomic heavy chain variable region there are three segments, V, D, and J, which recombine randomly, in a process called VDJ rearrangement to produce a unique variable region in the immunoglobulin of each B-cell. Similar rearrangements occur for the light chain variable region except that there are only two segments involved, V and J. After complete rearrangement, the B-cell reaches the IgM+ immature stage in the bone marrow. These immature B-cells present a membrane bound IgM, i.e., BCR, on their surface and migrate to the spleen, where they are called transitional B cells. Some of these cells differentiate into mature B lymphocytes. Mature B-cells expressing the BCR on their surface circulate the blood and lymphatic system performing the role of immune surveillance. They do not produce soluble antibodies until they become fully activated. Each B-cell has a unique receptor protein that will bind to one particular antigen. Once a B-cell encounters its antigen and receives an additional signal from a T helper cell, it can further differentiate into either a plasma B-cell expressing and secreting soluble antibodies or a memory B-cell.

In the context of the present disclosure, the term “B-cell” refers to any B lymphocyte which presents a fully rearranged, i.e., a mature, BCR on its surface. For example, a B-cell in the context of the present invention may be an immature or a mature B-cell. In some cases, the B-cell is a naïve B-cell, i.e., a B-cell that has not been exposed to the antigen specifically recognized by the BCR on the surface of said B-cell. In some embodiments, the B-cells are CD19+ B-cells, i.e., express CD19 on their surface. In some cases, the B-cells in the context of the present invention are CD19+ B-cells and express a fully rearranged BCR on their surface. The B-cells may also be CD20+ or CD21+ B-cells. In some cases, the CD20+ or CD21+ B-cells carry a BCR on their surface. In some embodiments, the B-cells are memory B-cells, such as IgG+ memory B cells.

A suitable BTN3A ectodomain polypeptide stimulates APC activity (activates APCs) and thereby induces an increased immune response, e.g., increased T cell activity. Activation of an APC is associated with (i) an increased secretion of one or more helper cytokines (e.g., two or more, three or more, four or more, five or more helper cytokines) from the APC, (ii) an increase in the production of one or more costimulatory molecules (e.g., two or more, three or more, four or more, five or more costimulatory molecules), and/or (iii) an increase in one or more downstream effector functions (e.g., two or more, three or more, four or more, five or more downstream effector functions) of APCs (to elicit an immune response). Thus, one or more assays (e.g., one assay, two or more assays, 2 assays, 3 assays, etc.) can be used to determine whether APCs have been activated (e.g., after contacting APCs and/or monocytes with a subject BTN3A ectodomain polypeptide). For example, suitable assays include but are not limited to: (i) assays that measure secretion of one or more helper cytokines (e.g., two or more, three or more, four or more, five or more helper cytokines) from APCs, (ii) assays that measure the production of one or more costimulatory molecules (e.g., two or more, three or more, four or more, five or more costimulatory molecules) by APCs, and (iii) assays that measure one or more downstream effector functions (e.g., two or more, three or more, four or more, five or more downstream effector functions) of APCs (to elicit an immune response). For each of the three assay types listed above, an increase is associated with an increase in APC activity (e.g., an increase relative to the measured level(s) in the absence of contact with the BTN3A ectodomain polypeptide; an increase relative to the measured level(s) prior to contact with the BTN3A ectodomain polypeptide; an increase relative to the measured level(s) after contact with a control molecule, e.g., a polypeptide that is not a BTN3A ectodomain polypeptide; and the like).

Examples of “helper cytokines,” the secretion of which can be stimulated (increased) when an APC is activated, include but are not limited to: IL1, IL-4, IL-6, IL-8, IL-10, IL-12, IL-18, IL-23, IL-27, IP-10, RANTES, IFN-alpha, and TGF-beta. Thus, in some cases, an active APC secretes an increased amount of one or more of (e.g., two or more, three or more, four or more, or five or more of): IL1, IL-4, IL-6, IL-8, IL-10, IL-12, IL-18, IL-23, IL-27, IP-10, RANTES, IFN-alpha, and TGF-beta after being contacted with a subject BTN3A ectodomain polypeptide. Therefore, in some cases, activation of an APC can be determined by measuring one or more of (e.g., two or more, three or more, four or more, or five or more of): IL1, IL-4, IL-6, IL-8, IL-10, IL-12, IL-18, IL-23, IL-27, IP-10, RANTES, IFN-alpha, and TGF-beta.

Examples of “costimulatory molecules,” the production of which can be stimulated (increased) when an APC is activated, included but are not limited to: CD80 (B7-1), CD86 (B7-2), ICOSL (B7-H2), CD40, OX40L, PD-1L, PD-2L, B7-H3, BTLA, 4-1BB-L, CD134L, CD70, CD27, CD30-L, LIGHT, SLAM, CD48, CD58, CD155, CD112, TIM4, GITRL, TL1A, and HVEM. Thus, in some cases, an active APC produces an increased amount of one or more of (e.g., two or more, three or more, four or more, or five or more of CD80 (B7-1), CD86 (B7-2), ICOSL (B7-H2), CD40, OX40L, PD-1L, PD-2L, B7-H3, BTLA, 4-1BB-L, CD134L, CD70, CD27, CD30-L, LIGHT, SLAM, CD48, CD58, CD155, CD112, TIM4, GITRL, TL1A, and HVEM after being contacted with a subject BTN3A ectodomain polypeptide. Therefore, in some cases, activation of an APC can be determined by measuring one or more of (e.g., two or more, three or more, four or more, or five or more of): CD80 (B7-1), CD86 (B7-2), ICOSL (B7-H2), CD40, OX40L, PD-1L, PD-2L, B7-H3, BTLA, 4-1BB-L, CD134L, CD70, CD27, CD30-L, LIGHT, SLAM, CD48, CD58, CD155, CD112, TIM4, GITRL, TL1A, and HVEM.

Examples of “downstream effector functions” of an APC, which function can be stimulated (increased) when an APC is activated, include but are not limited to: antigen-specific priming of CD8+ cells (e.g., ability to prime a naive T-cell into an antigen specific effector cell); endocytosis and/or phagocytosis of antigen positive (+) cells; migration and/or trafficking to sites of inflammation and/or to draining lymph nodes (to present antigen); and engagement in antibody-dependent cell-mediated cytotoxicity. Therefore, in some cases, activation of an APC can be determined by measuring one or more of (e.g., two or more, three or more, four or more, or five or more of): antigen-specific priming of CD8+ cells (e.g., ability to prime a naive T-cell into an antigen specific effector cell) by the APC; endocytosis and/or phagocytosis of antigen positive (+) cells by the APC; migration and/or trafficking to sites of inflammation and/or to draining lymph nodes (to present antigen) by the APC; and engagement in antibody-dependent cell-mediated cytotoxicity by the APC.

In some cases, a BTN3A ectodomain polypeptide will be able to produce an increase in APC activity (i.e., will be able to activate APCs) as measured using one or more (e.g., two or more, or all three) of the three listed assay types above (e.g., assays that measure secretion of one or more helper cytokines from APCs, assays that measure the production of one or more costimulatory molecules by APCs, and/or assays that measure one or more downstream effector functions of APCs), compared to activity in the absence of contact with a BTN3A ectodomain polypeptide, compared to activity prior to contact with the BTN3A ectodomain polypeptide, or compared to activity after contacting APCs and/or monocytes with a control molecule (e.g., a polypeptide that is not a BTN3A ectodomain polypeptide).

Thus, for example, in some cases, a BTN3A ectodomain polypeptide can be identified by using one or more assays (e.g., two or more assays, or all three assays) selected from: (i) an assay that measures secretion of one or more helper cytokines (e.g., two or more, three or more, four or more, five or more helper cytokines) from APCs, (ii) an assay that measures the production of one or more costimulatory molecules (e.g., two or more, three or more, four or more, five or more costimulatory molecules) by APCs, and (iii) an assay that measures one or more downstream effector functions (e.g., two or more, three or more, four or more, five or more downstream effector functions) of APCs to elicit an immune response. For example, in some cases a candidate BTN3A ectodomain polypeptide can be determined to be a BTN3A ectodomain polypeptide if the candidate BTN3A ectodomain polypeptide elicits (upon contact with an APC and/or a monocyte) one or more (e.g., two or more, or all three) of: (i) an increase in secretion of one or more helper cytokines (e.g., two or more, three or more, four or more, five or more helper cytokines) from APCs, (ii) an increase in the production of one or more costimulatory molecules (e.g., two or more, three or more, four or more, five or more costimulatory molecules) by APCs, and (iii) an increase in one or more downstream effector functions (e.g., two or more, three or more, four or more, five or more downstream effector functions) of APCs to elicit an immune response; when compared to measured control level(s) (e.g., level(s) measured in the absence of contact, e.g., level(s) measured prior to contacting APCs and/or monoctyes with a candidate BTN3A ectodomain polypeptide; and/or level(s) measured after contacting APCs and/or monoctyes with a control molecule, e.g., a polypeptide that is not a BTN3A ectodomain polypeptide).

In some cases, a BTN3A ectodomain polypeptide can be identified by using two or more assays (e.g., all three assays) selected from: (i) an assay that measures secretion of one or more helper cytokines (e.g., two or more, three or more, four or more, five or more helper cytokines) from APCs, (ii) an assay that measures the production of one or more costimulatory molecules (e.g., two or more, three or more, four or more, five or more costimulatory molecules) by APCs, and (iii) an assay that measures one or more downstream effector functions (e.g., two or more, three or more, four or more, five or more downstream effector functions) of APCs to elicit an immune response. For example, in some cases a candidate BTN3A ectodomain polypeptide can be determined to be a BTN3A ectodomain polypeptide if the candidate BTN3A ectodomain polypeptide elicits (upon contact with an APC and/or a monocyte) two or more (e.g., all three) of: (i) an increase in secretion of one or more helper cytokines (e.g., two or more, three or more, four or more, five or more helper cytokines) from APCs, (ii) an increase in the production of one or more costimulatory molecules (e.g., two or more, three or more, four or more, five or more costimulatory molecules) by APCs, and (iii) an increase in one or more downstream effector functions (e.g., two or more, three or more, four or more, five or more downstream effector functions) of APCs to elicit an immune response; when compared to measured control level(s) (e.g., level(s) measured in the absence of contact, e.g., level(s) measured prior to contacting APCs and/or monoctyes with a candidate BTN3A ectodomain polypeptide; and/or level(s) measured after contacting APCs and/or monoctyes with a control molecule, e.g., a polypeptide that is not a BTN3A ectodomain polypeptide).

In some cases, a BTN3A ectodomain polypeptide can be identified by using the following three assays: (i) an assay that measures secretion of one or more helper cytokines (e.g., two or more, three or more, four or more, five or more helper cytokines) from APCs, (ii) an assay that measures the production of one or more costimulatory molecules (e.g., two or more, three or more, four or more, five or more costimulatory molecules) by APCs, and (iii) an assay that measures one or more downstream effector functions (e.g., two or more, three or more, four or more, five or more downstream effector functions) of APCs to elicit an immune response. For example, in some cases a candidate BTN3A ectodomain polypeptide can be determined to be a BTN3A ectodomain polypeptide if the candidate BTN3A ectodomain polypeptide elicits (upon contact with an APC and/or a monocyte): (i) an increase in secretion of one or more helper cytokines (e.g., two or more, three or more, four or more, five or more helper cytokines) from APCs, (ii) an increase in the production of one or more costimulatory molecules (e.g., two or more, three or more, four or more, five or more costimulatory molecules) by APCs, and (iii) an increase in one or more downstream effector functions (e.g., two or more, three or more, four or more, five or more downstream effector functions) of APCs to elicit an immune response; when compared to measured control level(s) (e.g., level(s) measured in the absence of contact, e.g., level(s) measured prior to contacting APCs and/or monoctyes with a candidate BTN3A ectodomain polypeptide; and/or level(s) measured after contacting APCs and/or monoctyes with a control molecule, e.g., a polypeptide that is not a BTN3A ectodomain polypeptide).

In some cases, a BTN3A ectodomain polypeptide induces an increase in the secretion of one or more helper cytokines (e.g., two or more, three or more, four or more, five or more helper cytokines) from APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the secretion of helper cytokines from control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.).

In some cases, a BTN3A ectodomain polypeptide induces an increase in the production of one or more costimulatory molecules (e.g., two or more, three or more, four or more, five or more costimulatory molecules) by APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the production of costimulatory molecules from control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.).

In some cases, a high affinity BTN3A ectodomain polypeptide induces an increase in one or more of the downstream effector functions of APCs (e.g., to elicit an immune response) by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the downstream effector functions of APCs (to elicit an immune response) of control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.).

In some cases, a BTN3A ectodomain polypeptide (a) induces an increase in the secretion of one or more helper cytokines (e.g., two or more, three or more, four or more, five or more helper cytokines) from APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the secretion of helper cytokines from control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.); and (b) induces an increase in the production of one or more costimulatory molecules (e.g., two or more, three or more, four or more, five or more costimulatory molecules) by APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the production of costimulatory molecules from control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.).

In some cases, a BTN3A ectodomain polypeptide (a) induces an increase in the secretion of one or more helper cytokines (e.g., two or more, three or more, four or more, five or more helper cytokines) from APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the secretion of helper cytokines from control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.); and (b) induces an increase in one or more of the downstream effector functions of APCs (e.g., to elicit an immune response) by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the downstream effector functions of APCs (to elicit an immune response) of control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.).

In some cases, a BTN3A ectodomain polypeptide (a) induces an increase in the production of one or more costimulatory molecules (e.g., two or more, three or more, four or more, five or more costimulatory molecules) by APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the production of costimulatory molecules from control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.); and (b) induces an increase in one or more of the downstream effector functions of APCs (e.g., to elicit an immune response) by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the downstream effector functions of APCs (to elicit an immune response) of control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.).

In some cases, a BTN3A ectodomain polypeptide (a) induces an increase in the secretion of one or more helper cytokines (e.g., two or more, three or more, four or more, five or more helper cytokines) from APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the secretion of helper cytokines from control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.); (b) induces an increase in the production of one or more costimulatory molecules (e.g., two or more, three or more, four or more, five or more costimulatory molecules) by APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the production of costimulatory molecules from control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.); and (c) induces an increase in one or more of the downstream effector functions of APCs (e.g., to elicit an immune response) by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the downstream effector functions of APCs (to elicit an immune response) of control cells (e.g., APCs and/or monoctyes that have not been contacted with a BTN3A ectodomain polypeptide, e.g., APCs and/or monoctyes prior to contact with a BTN3A ectodomain polypeptide, APCs and/or monoctyes contacted with a molecule that is not a BTN3A ectodomain polypeptide, etc.).

### BTN3A Proteins

A wild type BTN3A protein has a transmembrane domain and is expressed on the cell surface. A wild type BTN3A protein, expressed on the surface of a first cell, specifically binds to its counter receptor (e.g., LTβR, FLT1, HLA-E, CD163, and/or ROR2), expressed on the surface of a second cell. BTN3A proteins (e.g., BTN3A1, BTN3A2, BTN3A3) include those from any species, e.g., a mammalian BTN3A protein, a rodent BTN3A protein, a primate BTN3A protein, a rat BTN3A protein, a mouse BTN3A protein, a pig BTN3A protein, a cow BTN3A protein, a sheep BTN3A protein, a rabbit BTN3A protein, a dog BTN3A protein, a human BTN3A protein, etc. Sequences for various wild type BTN3A polypeptide sequences (e.g., canine, bovine, sheep, equine, porcine, rodent, mouse, rat, feline, primate, monkey, ape, chimpanzee, and the like) can easily be found and are readily available to one of ordinary skill in the art. For example, isoforms of the human BTN3A1, BTN3A2, and BTN3A3 proteins (set forth as SEQ ID NOs: 1-7) are listed below.

Wild Type Human BTN3A1

(also known as CD277, BTN3.1, BT3.1, BTF5, and
″butyrophilin, subfamily 3, member A1″)
NP_008979.3 (isoform a)
(SEQ ID NO: 1)
MKMASFLAFLLLNFRVCLLLLQLLMPHSAQFSVLGPSGPILAMVGEDAD
LPCHLFPTMSAETMELKWVSSSLRQVVNVYADGKEVEDRQSAPYRGRTS
ILRDGITAGKAALRIHNVTASDSGKYLCYFQDGDFYEKALVELKVAALG
SDLHVDVKGYKDGGIHLECRSTGWYPQPQIQWSNNKGENIPTVEAPVVA
DGVGLYAVAASVIMRGSSGEGVSCTIRSSLLGLEKTASISIADPFFRSA
QRWIAALAGTLPVLLLLLGGAGYFLWQQQEEKKTQFRKKKREQELREMA
WSTMKQEQSTRVKLLEELRWRSIQYASRGERHSAYNEWKKALFKPADVI
LDPKTANPILLVSEDQRSVQRAKEPQDLPDNPERFNWHYCVLGCESFIS
GRHYWEVEVGDRKEWHIGVCSKNVQRKGWVKMTPENGFWTMGLTDGNKY
RTLTEPRTNLKLPKPPKKVGVFLDYETGDISFYNAVDGSHIHTFLDVSF
SEALYPVFRILTLEPTALTICPA bold: example of a
transmembrane domain (as discussed below)-amino
acids 248-271 underline: example of a sequence
that includes a BTN3A1 ectodomain-amino acids 30-
246 (SEQ ID NO: 10)
NP_919423.1 (isoform b)
(SEQ ID NO: 2)
MKMASFLAFLLLNFRVCLLLLQLLMPHSAQFSVLGPSGPILAMVGEDAD
LPCHLFPTMSAETMELKWVSSSLRQVVNVYADGKEVEDRQSAPYRGRTS
ILRDGITAGKAALRIHNVTASDSGKYLCYFQDGDFYEKALVELKVAALG
SDLHVDVKGYKDGGIHLECRSTGWYPQPQIQWSNNKGENIPTVEAPVVA
DGVGLYAVAASVIMRGSSGEGVSCTIRSSLLGLEKTASISIADPFFRSA
QRWIAALAGTLPVLLLLLGGAGYFLWQQQEEKKTQFRKKKREQELREMA
WSTMKQEQSTRVKLLEELRWRSIQYASRGERHSAYNEWKKALFKPGEEM
LQMRLHFVK bold: example of a transmembrane domain
(as discussed below)-amino acids 248-271
underline: example of a sequence that includes a
BTN3A1 ectodomain-amino acids 30-246 (SEQ ID
NO: 11)
NP_001138481.1 (isoform d)
(SEQ ID NO: 3)
MKMASFLAFLLLNFRVCLLLLQLLMPHSAQFSVLGPSGPILAMVGEDADL
PCHLFPTMSAETMELKWVSSSLRQVVNVYADGKEVEDRQSAPYRGRTSIL
RDGITAGKAALRIHNVTASDSGKYLCYFQDGDFYEKALVELKVAALGSDL
HVDVKGYKDGGIHLECRSTGWYPQPQIQWSNNKGENIPTVEAPVVADGVG
LYAVAASVIMRGSSGEGVSCTIRSSLLGLEKTASISIADPFFRSAQRWIA
ALAGTLPVLLLLLGGAGYFLWQQQEEKKTQFRKKKREQELREMAWSTMKQ
EQSTRVKLLEELRWRSIQYASRGERHSAYNEWKKALFKPGPPIGQTQQQT
RGQGSPVALSQESAQRTDSWGPEEGGESA bold: example of a
transmembrane domain (as discussed below)-amino
acids 248-271 underline: example of a sequence
that includes a BTN3A1 ectodomain- amino acids
30-246 (SEQ ID NO: 12)

### Wild Type Human BTN3A2

(also known as BTN3.2, BT3.2, BTF3, BTF4,
″butyrophilin, subfamily 3, member A2″)
NP_001184175.1; NP_001184176.1; NP_008978.2
(isoform a)
(SEQ ID NO: 4)
MKMASSLAFLLLNFHVSLLLVQLLTPCSAQFSVLGPSGPILAMVGEDAD
LPCHLFPTMSAETMELKWVSSSLRQVVNVYADGKEVEDRQSAPYRGRTS
ILRDGITAGKAALRIHNVTASDSGKYLCYFQDGDFYEKALVELKVAALG
SNLHVEVKGYEDGGIHLECRSTGWYPQPQIQWSNAKGENIPAVEAPVVA
DGVGLYEVAASVIMRGGSGEGVSCIIRNSLLGLEKTASISIADPFFRSA
QPWIAALAGTLPILLLLLAGASYFLWRQQKEITALSSEIESEQEMKEMG
YAATEREISLRESLQEELKRKKIQYLTRGEESSSDTNKSA bold:
example of a transmembrane domain (as discussed
below)-amino acids 248-271 underline: example of
a sequence that includes a BTN3A2 ectodomain-
amino acids 30-246 (SEQ ID NO: 13)
NP_001184177.1 (isoform b)
(SEQ ID NO: 5)
MGIPRAQFSVLGPSGPILAMVGEDADLPCHLFPTMSAETMELKWVSSSL
RQVVNVYADGKEVEDRQSAPYRGRTSILRDGITAGKAALRIHNVTASDS
GKYLCYFQDGDFYEKALVELKVAALGSNLHVEVKGYEDGGIHLECRSTG
WYPQPQIQWSNAKGENIPAVEAPVVADGVGLYEVAASVIMRGGSGEGVS
CIIRNSLLGLEKTASISIADPFFRSAQPWIAALAGTLPILLLLLAGASY
FLWRQQKEITALSSEIESEQEMKEMGYAATEREISLRESLQEELKRKKI
QYLTRGEESSSDTNKSA bold: example of a trans-
membrane domain (as discussed below)-amino acids
225-248 underline: example of a sequence that
includes a BTN3A2 ectodomain-amino acids 7-223
(SEQ ID NO: 14)

Wild Type Human BTN3A3

(also known as BTN3.3, BTF3, ″butyrophilin,
subfamily 3, member A3″) NP_008925.1 (isoform a)
(SEQ ID NO: 6)
MKMASSLAFLLLNFHVSLFLVQLLTPCSAQFSVLGPSGPILAMVGEDAD
LPCHLFPTMSAETMELRWVSSSLRQVVNVYADGKEVEDRQSAPYRGRTS
ILRDGITAGKAALRIHNVTASDSGKYLCYFQDGDFYEKALVELKVAALG
SDLHIEVKGYEDGGIHLECRSTGWYPQPQIKWSDTKGENIPAVEAPVVA
DGVGLYAVAASVIMRGSSGGGVSCIIRNSLLGLEKTASISIADPFFRSA
QPWIAALAGTLPISLLLLAGASYFLWRQQKEKIALSRETEREREMKEMG
YAATEQEISLREKLQEELKWRKIQYMARGEKSLAYHEWKMALFKPADVI
LDPDTANAILLVSEDQRSVQRAEEPRDLPDNPERFEWRYCVLGCENFTS
GRHYWEVEVGDRKEWHIGVCSKNVERKKGWVKMTPENGYWTMGLTDGNK
YRALTEPRTNLKLPEPPRKVGIFLDYETGEISFYNATDGSHIYTFPHAS
FSEPLYPVFRILTLEPTALTICPIPKEVESSPDPDLVPDHSLETPLTPG
LANESGEPQAEVTSLLLPAHPGAEVSPSATTNQNHKLQARTEALY
bold: example of a transmembrane domain (as
discussed below)-amino acids 248-271 underline:
example of a sequence that includes a BTN3A3
ectodomain-amino acids 30-246 (SEQ ID NO: 15)
NP_932078.2 (isoform b)
(SEQ ID NO: 7)
MVGEDADLPCHLFPTMSAETMELRWVSSSLRQVVNVYADGKEVEDRQSA
PYRGRTSILRDGITAGKAALRIHNVTASDSGKYLCYFQDGDFYEKALVE
LKVAALGSDLHIEVKGYEDGGIHLECRSTGWYPQPQIKWSDTKGENIPA
VEAPVVADGVGLYAVAASVIMRGSSGGGVSCIIRNSLLGLEKTASISIA
DPFFRSAQPWIAALAGTLPISLLLLAGASYFLWRQQKEKIALSRETERE
REMKEMGYAATEQEISLREWRKIQYMARGEKSLAYHEWKMALFKPADVI
LDPDTANAILLVSEDQRSVQRAEEPRDLPDNPERFEWRYCVLGCENFTS
GRHYWEVEVGDRKEWHIGVCSKNVERKKGWVKMTPENGYWTMGLTDGNK
YRALTEPRTNLKLPEPPRKVGIFLDYETGEISFYNATDGSHIYTFPHAS
FSEPLYPVFRILTLEPTALTICPIPKEVESSPDPDLVPDHSLETPLTPG
LANESGEPQAEVTSLLLPAHPGAEVSPSATTNQNHKLQARTEALY
bold: example of a transmembrane domain (as
discussed below)-amino acids 206-229 underline:
example of a sequence that includes a BTN3A3
ectodomain-amino acids 1-204 (SEQ ID NO: 16)

Sequences for various additional wild type BTN3A polypeptide sequences (e.g., BTN3A1, BTN3A2, and BTN3A3 polypeptide sequences) (e.g., canine, bovine, sheep, equine, porcine, rodent, mouse, rat, feline, primate, monkey, ape, chimpanzee, and the like) can easily be found and are readily available to one of ordinary skill in the art.

### BTN3A Ectodomain Polypeptides

Because the ectodomains of wild type BTN3A1, BTN3A2, and BTN3A3 are highly conserved, the term “BTN3A” is used herein as a generic term that encompasses BTN3A subfamily members. For example, the term “BTN3A polypeptide” encompasses “BTN3A1 polypeptide”, “BTN3A2 polypeptide”, and “BTN3A3 polypeptide.” Likewise, the term “BTN3A ectodomain” encompasses “BTN3A1 ectodomain”, “BTN3A2 ectodomain”, and “BTN3A3 ectodomain.” Thus, referral to a BTN3A ectodomain encompasses BTN3A1, BTN3A2, and BTN3A3 ectodomains. Referral to a BTN3A ectodomain polypeptide encompasses a BTN3A ectodomain polypeptide having a BTN3A1, BTNA2, or BTNA3 ectodomain (unless otherwise explicitly defined).

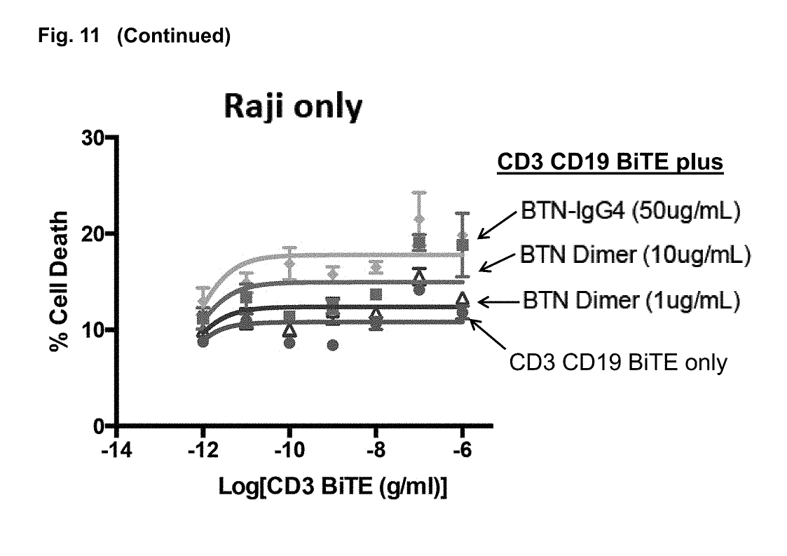

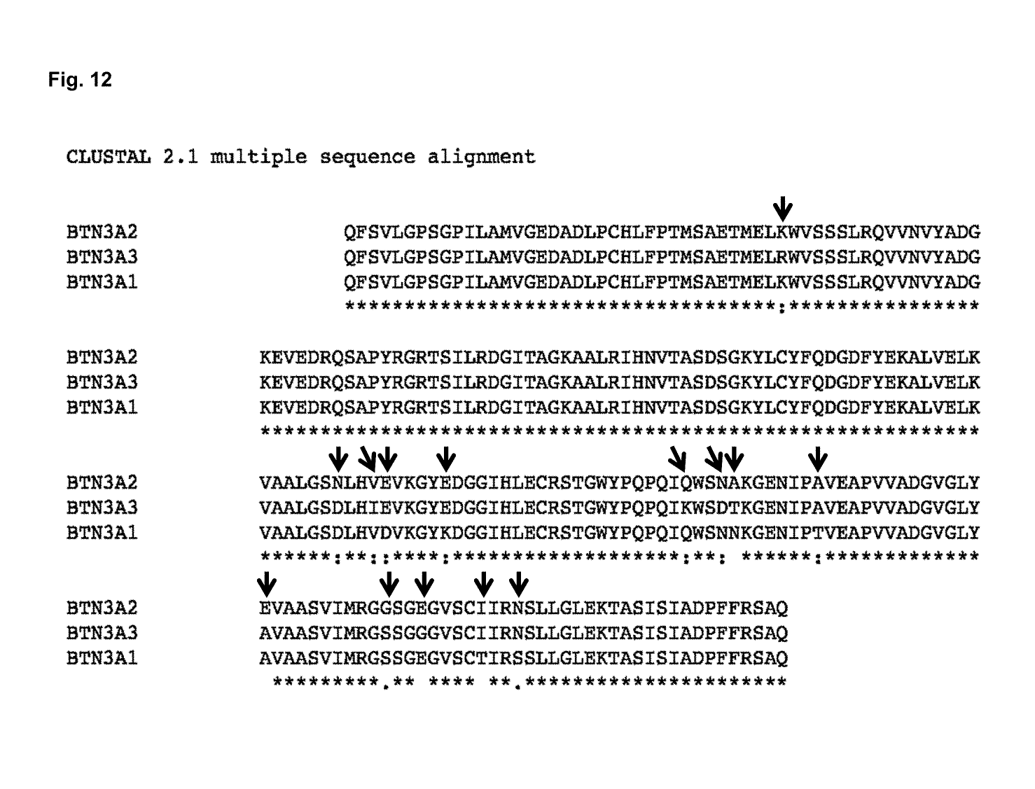

To illustrate the high level of sequence conservation among the ectodomains of the wild type human BTN3A proteins, alignments (see FIG. 12) from the examples above (underlined regions above of sequences that include a BTN3A1, BTNA2, or BTNA3 ectodomain) reveal that the amino acid sequence set forth as SEQ ID NO: 10 (an example of a sequence that includes a BTN3A1 ectodomain) has 96% amino acid sequence identity with the amino acid sequence set forth as SEQ ID NO: 13 (an example of a sequence that includes a BTN3A2 ectodomain) over the entire 217 amino acids. Likewise, the amino acid sequence set forth as SEQ ID NO: 10 (an example of a sequence that includes a BTN3A1 ectodomain) has 95% amino acid sequence identity with the amino acid sequence set forth as SEQ ID NO: 15 (an example of a sequence that includes a BTN3A3 ectodomain) over the entire 217 amino acids. Similarly, the amino acid sequence set forth as SEQ ID NO: 13 (an example of a sequence that includes a BTN3A2 ectodomain) has 96% amino acid sequence identity with the amino acid sequence set forth as SEQ ID NO: 15 (an example of a sequence that includes a BTN3A3 ectodomain) over the entire 217 amino acids. A multiple sequence alignment of the 3 sequences is presented as FIG. 12.

A subject “BTN3A ectodomain polypeptide” includes a BTN3A ectodomain (i.e., a portion of a BTN3A protein that is sufficient to activate an APC—induce/increase APC activity) and lacks a BTN3A transmembrane domain (i.e. a transmembrane domain of a BTN3A protein). In a wild type BTN3A protein, the ectodomain is between the signal sequence and the transmembrane domain. The BTN3A ectodomain portion of a subject BTN3A ectodomain polypeptide can include all of the amino acids between the signal sequence and the transmembrane domain of the wild type protein, or a portion thereof (or can included one or more amino acid mutations compared to a corresponding wild type BTN3A protein) that is sufficient to stimulate (increase, induce) APC activity (i.e., activate an APC).

In some cases, a BTN3A ectodomain polypeptide includes a BTN3A ectodomain of a BTN3A1 protein. In some cases, a BTN3A ectodomain polypeptide includes a BTN3A ectodomain of a BTN3A2 protein. In some cases, a BTN3A ectodomain polypeptide includes a BTN3A ectodomain of a BTN3A3 protein.

In some cases a BTN3A ectodomain polypeptide includes a signal sequence (e.g., a signal sequence from a wild type BTN3A protein, e.g., BTN3A1, BTN3A2, or BTN3A3). In some cases a BTN3A ectodomain polypeptide includes a heterologous signal sequence (i.e., a signal sequence not associated with a BTN3A ectodomain in nature) (e.g., a Gp67 signal peptide, an IL-2 signal peptide, and the like). In some cases a BTN3A ectodomain polypeptide does not include a signal sequence.

### Ectodomain

In some cases, the BTN3A ectodomain of a BTN3A ectodomain polypeptide includes an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in any of SEQ ID NOs: 10, 13, and 15. For example, in some cases, the BTN3A ectodomain of a BTN3A ectodomain polypeptide includes an amino acid sequence having 70% or more sequence identity (e.g., 80% or more, 85% or more, 90% or more, 95% or more, 98% or more, 99% or more, 99.5% or more, or 100% sequence identity) with the amino acid sequence of the BTN3A1 ectodomain sequence set forth in SEQ ID NO: 10. In some cases, the BTN3A ectodomain of a BTN3A ectodomain polypeptide includes an amino acid sequence having 70% or more sequence identity (e.g., 80% or more, 85% or more, 90% or more, 95% or more, 98% or more, 99% or more, 99.5% or more, or 100% sequence identity) with the amino acid sequence of the BTN3A2 ectodomain sequence set forth in SEQ ID NO: 13. In some cases, the BTN3A ectodomain of a BTN3A ectodomain polypeptide includes an amino acid sequence having 70% or more sequence identity (e.g., 80% or more, 85% or more, 90% or more, 95% or more, 98% or more, 99% or more, 99.5% or more, or 100% sequence identity) with the amino acid sequence of the BTN3A3 ectodomain sequence set forth in SEQ ID NO: 15.

In some cases, a BTN3A ectodomain includes one of the immunoglobulin domains of a wild type BTN3A protein (or a functionally similar polypeptide having one or more amino acid mutations relative to a corresponding wild type BTN3A protein). In some cases, a BTN3A ectodomain includes both of the immunoglobulin domains of a wild type BTN3A protein (or a functionally similar polypeptide having one or more amino acid mutations relative to a corresponding wild type BTN3A protein). The immunoglobulin domains of the ectodomain of the wild type BTN3A protein can readily be identified by one of ordinary skill in the art. For example, a scan of the wild type human BTN3A1, BTN3A2, and BTN3A3 amino acid sequences set forth in SEQ ID NOs: 1, 4, and 6, respectively reveals that the region from amino acids 26-236 (BTN3A1), 26-228 (BTN3A2), or 26-236 (BTN3A3) contains two immunoglobulin domains (Table 1).

TABLE 1
Immunoglobulin domains of wild type BTN3A1, BTN3A2, and
BTN3A3 identified by various sequence analysis software.

| | | |
|---|---|---|
| Amino acids | Domain | Database |

BTN3A1 - SEQ ID NO: 1

| | | |
|---|---|---|
| 26-139 | Immunoglobulin-like domain | PROSITE |
| 164-236 | Immunoglobulin-like domain | |
| 30-143 | Immunoglobulin V-set domain | Pfam |
| 154-231 | CD80-like C2-set immunoglobulin domain | |
| 37-144 | Immunoglobulin (IG) domain | SMART |
| 36-145, 161-186 | CATH Domain 3d9aH01 (Ig heavy chain V region 3-6 | GENE3D/CATH |
| | (Ig heavy chain V region M315)) | |
| 37-143 | IG_like; Immunoglobulin like | NCBI |
| 45-144 | Ig_MOG_like; Immunoglobulin (Ig)-like domain of | |
| | myelin oligodendrocyte glycoprotein (MOG) | |
| 164-228 | Ig; Immunoglobulin domain | |
| 30-139 | Ig-like V-type | UniProt |
| 145-236 | Ig-like V-type | |
| 30-254 | Extracellular | |

BTN3A2 - SEQ ID NO: 4

| | | |
|---|---|---|
| 26-139 | Ig-like | PROSITE |
| 30-143 | Immunoglobulin V-set domain | Pfam |
| 37-144 | Immunoglobulin (IG) domain | SMART |
| 36-145, 157-183 | CATH Domain 3d9aH01(Ig heavy chain V region 3-6 | GENE3D/CATH |
| | (Ig heavy chain V region M315)) | |
| 37-143 | IG_like; Immunoglobulin like | NCBI |
| 45-144 | Ig_MOG_like; Immunoglobulin (Ig)-like domain of | |
| | myelin oligodendrocyte glycoprotein (MOG) | |
| | Ig (“Immunoglobulin domain; cl11960”) | |
| 164-228 | Ig; Immunoglobulin domain | |
| 30-139 | Ig-like V-type | UniProt |
| 30-248 | Extracellular | |

BTN3A3 - SEQ ID NO: 6

| | | |
|---|---|---|
| 26-139 | Ig-like | PROSITE |
| 164-236 | Ig-like | |
| 30-143 | Immunoglobulin V-set domain | Pfam |
| 37-144 | IG | SMART |
| 36-145, 158-182 | CATH Domain 3d9aH01(Ig heavy chain V region 3-6 | GENE3D/CATH |
| | (Ig heavy chain V region M315)) | |
| 37-143 | IG_like; Immunoglobulin like | NCBI |
| 45-144 | Ig_MOG_like; Immunoglobulin (Ig)-like domain of | |
| | myelin oligodendrocyte glycoprotein (MOG) | |
| | Ig (“Immunoglobulin domain; cl11960”) | |
| 30-139 | Ig-like V-type | UniProt |
| 145-236 | Ig-like V-type | |
| 30-248 | Extracellular | |

As can be seen in Table 1, the ectodomain of the wilde type BTN3A1 protein (isoform a) set forth in SEQ ID NO: 1 includes two immunoglobulin-like domains, the first located at amino acids 26-145 (e.g., 26-139, 30-143, 37-144, 36-145, 37-143, 45-144, 30-139) (of isoform a above) and the second located at amino acids 145-236 (e.g., 164-228, 164-236, 154-231, 161-186, 145-236) (of isoform a above). The ectodomain of the wilde type BTN3A2 protein (isoform a) set forth in SEQ ID NO: 4 includes two immunoglobulin-like domains, the first located at amino acids 26-145 (e.g., 26-139, 30-143, 37-144, 36-145, 37-143, 45-144, 30-139) (of isoform a above) and the second located at amino acids 157-228 (e.g., 157-183, 164-228) (of isoform a above). The ectodomain of the wilde type BTN3A3 protein (isoform a) set forth in SEQ ID NO: 6 includes two immunoglobulin-like domains, the first located at amino acids 26-145 (e.g., 26-139, 30-143, 37-144, 36-145, 37-143, 45-144, 30-139) (of isoform a above) and the second located at amino acids 145-236 (e.g., 164-236, 158-182, 145-236) (of isoform a above).

In some cases, the BTN3A ectodomain of a BTN3A ectodomain polypeptide includes an immunoglobulin domain (e.g., amino acids 26-145, 26-139, 30-143, 37-144, 36-145, 37-143, 45-144, 30-139, 145-236, 164-228, 164-236, 154-231, 161-186, and/or 145-236 of the wild type BTN3A1 amino acid sequence set forth as SEQ ID NO: 1, or the corresponding region of another wild type BTN3A1 polypeptide; amino acids 26-145, 26-139, 30-143, 37-144, 36-145, 37-143, 45-144, 30-139, 157-228, 157-183, and/or 164-228 of the wild type BTN3A2 amino acid sequence set forth as SEQ ID NO: 4, or the corresponding region of another wild type BTN3A2 polypeptide; amino acids 26-145, 26-139, 30-143, 37-144, 36-145, 37-143, 45-144, 30-139, 164-236, 158-182, and/or 145-236 of the wild type BTN3A3 amino acid sequence set forth as SEQ ID NO: 6, or the corresponding region of another wild type BTN3A3 polypeptide)(e.g., an Immunoglobulin-like domain; an Immunoglobulin V-set domain; an Immunoglobulin (IG) domain, an IG_like domain, an Ig_MOG_like domain; an Ig-like V-type 1 domain, an CD80-like C2-set immunoglobulin domain, and Ig-like V-type 2 domain, and the like).

In some cases, the BTN3A ectodomain of a BTN3A ectodomain polypeptide includes two immunoglobulin domains. For example, in some cases, the BTN3A ectodomain of a BTN3A ectodomain polypeptide includes (i) a first immunoglobulin domain (e.g., amino acids 26-145, 26-139, 30-143, 37-144, 36-145, 37-143, 45-144, and/or 30-139 of the wild type BTN3A1 amino acid sequence set forth as SEQ ID NO: 1, or to the corresponding region of another wild type BTN3A1 polypeptide; amino acids 26-145, 26-139, 30-143, 37-144, 36-145, 37-143, 45-144, and/or 30-139 of the wild type BTN3A2 amino acid sequence set forth as SEQ ID NO: 4, or to the corresponding region of another wild type BTN3A2 polypeptide; amino acids 26-145, 26-139, 30-143, 37-144, 36-145, 37-143, 45-144, and/or 30-139 of the wild type BTN3A3 amino acid sequence set forth as SEQ ID NO: 6, or to the corresponding region of another wild type BTN3A3 polypeptide)(e.g., an Immunoglobulin-like domain; an Immunoglobulin V-set domain; an Immunoglobulin (IG) domain, an IG_like domain, an Ig_MOG_like domain; an Ig-like V-type 1 domain, and the like); and also includes (ii) a second immunoglobulin domain (e.g., amino acids 145-236, 164-228, 164-236, 154-231, 161-186, and/or 145-236 of the wild type BTN3A1 amino acid sequence set forth as SEQ ID NO: 1, or the corresponding region of another wild type BTN3A1 polypeptide; amino acids 157-228, 157-183, and/or 164-228 of the wild type BTN3A2 amino acid sequence set forth as SEQ ID NO: 4, or the corresponding region of another wild type BTN3A2 polypeptide; amino acids 164-236, 158-182, and/or 145-236 of the wild type BTN3A3 amino acid sequence set forth as SEQ ID NO: 6, or the corresponding region of another wild type BTN3A3 polypeptide) (e.g., an immunoglobulin-like domain, an CD80-like C2-set immunoglobulin domain, and Ig-like V-type 2 domain, and the like).

Thus, in some cases, the BTN3A ectodomain of a BTN3A ectodomain polypeptide includes: (i) a first immunoglobulin domain that is classified as an Immunoglobulin-like domain; an Immunoglobulin V-set domain; an Immunoglobulin (IG) domain, an IG_like domain, an Ig_MOG_like domain; and/or an Ig-like V-type 1 domain, and (ii) a second immunoglobulin domain that is classified as an immunoglobulin-like domain, an CD80-like C2-set immunoglobulin domain, and/or an Ig-like V-type 2 domain.

In some cases, the BTN3A ectodomain of a BTN3A ectodomain polypeptide includes an immunoglobulin domain of an ectodomain of a wild type BTN3A protein (e.g., BTN3A1, BTN3A2, BTN3A3) (e.g., amino acids 26-145, 26-139, 30-143, 37-144, 36-145, 37-143, 45-144, 30-139 145-236, 164-228, 164-236, 154-231, 161-186, and/or 145-236 of the wild type BTN3A1 amino acid sequence set forth as SEQ ID NO: 1, or to the corresponding region of another wild type BTN3A1 polypeptide; amino acids 26-145, 26-139, 30-143, 37-144, 36-145, 37-143, 45-144, 30-139, 157-228, 157-183, and/or 164-228 of the wild type BTN3A2 amino acid sequence set forth as SEQ ID NO: 4, or to the corresponding region of another wild type BTN3A2 polypeptide; amino acids 26-145, 26-139, 30-143, 37-144, 36-145, 37-143, 45-144, 30-139, 164-236, 158-182, and/or 145-236 of the wild type BTN3A3 amino acid sequence set forth as SEQ ID NO: 6, or to the corresponding region of another wild type BTN3A3 polypeptide)(e.g., an Immunoglobulin-like domain; an Immunoglobulin V-set domain; an Immunoglobulin (IG) domain, an IG_like domain, an Ig_MOG_like domain; an Ig-like V-type 1 domain, an CD80-like C2-set immunoglobulin domain, and Ig-like V-type 2 domain, and the like.

In some cases, the BTN3A ectodomain of a BTN3A ectodomain polypeptide includes two immunoglobulin domains of an ectodomain of a wild type BTN3A protein. For example, in some cases, the BTN3A ectodomain of a BTN3A ectodomain polypeptide includes (i) a first immunoglobulin domain of an ectodomain of a wild type BTN3A protein (e.g., BTN3A1, BTN3A2, BTN3A3) (e.g., amino acids 26-145, 26-139, 30-143, 37-144, 36-145, 37-143, 45-144, and/or 30-139 of the wild type BTN3A1 amino acid sequence set forth as SEQ ID NO: 1, or to the corresponding region of another wild type BTN3A1 polypeptide; amino acids 26-145, 26-139, 30-143, 37-144, 36-145, 37-143, 45-144, and/or 30-139 of the wild type BTN3A2 amino acid sequence set forth as SEQ ID NO: 4, or the corresponding region of another wild type BTN3A2 polypeptide; amino acids 26-145, 26-139, 30-143, 37-144, 36-145, 37-143, 45-144, and/or 30-139 of the wild type BTN3A3 amino acid sequence set forth as SEQ ID NO: 6, or the corresponding region of another wild type BTN3A3 polypeptide)(e.g., an Immunoglobulin-like domain; an Immunoglobulin V-set domain; an Immunoglobulin (IG) domain, an IG_like domain, an Ig_MOG_like domain; an Ig-like V-type 1 domain, and the like); and also includes a second immunoglobulin domain of an ectodomain of a wild type BTN3A protein (e.g., BTN3A1, BTN3A2, BTN3A3) (e.g., amino acids 145-236, 164-228, 164-236, 154-231, 161-186, and/or 145-236 of the wild type BTN3A1 amino acid sequence set forth as SEQ ID NO: 1, or to the corresponding region of another wild type BTN3A1 polypeptide; amino acids 157-228, 157-183, and/or 164-228 of the wild type BTN3A2 amino acid sequence set forth as SEQ ID NO: 4, or the corresponding region of another wild type BTN3A2 polypeptide; amino acids 164-236, 158-182, and/or 145-236 of the wild type BTN3A3 amino acid sequence set forth as SEQ ID NO: 6, or the corresponding region of another wild type BTN3A3 polypeptide)(e.g., an immunoglobulin-like domain, an CD80-like C2-set immunoglobulin domain, and Ig-like V-type 2 domain, and the like).

In some cases, the BTN3A ectodomain of a BTN3A ectodomain polypeptide includes an amino acid sequence having 85% or more sequence identity (e.g., 90% or more, 95% or more, 98% or more, 99% or more, 99.2% or more, 99.5% or more, 99.8% or more, 99.9% or more, or 100% sequence identity) to an immunoglobulin domain of an ectodomain of a wild type BTN3A protein (e.g., BTN3A1, BTN3A2, BTN3A3)(e.g., amino acids 26-145, 26-139, 30-143, 37-144, 36-145, 37-143, 45-144, 30-139 145-236, 164-228, 164-236, 154-231, 161-186, and/or 145-236 of the wild type BTN3A1 amino acid sequence set forth as SEQ ID NO: 1, or to the corresponding region of another wild type BTN3A1 polypeptide; amino acids 26-145, 26-139, 30-143, 37-144, 36-145, 37-143, 45-144, 30-139, 157-228, 157-183, and/or 164-228 of the wild type BTN3A2 amino acid sequence set forth as SEQ ID NO: 4, or the corresponding region of another wild type BTN3A2 polypeptide; amino acids 26-145, 26-139, 30-143, 37-144, 36-145, 37-143, 45-144, 30-139, 164-236, 158-182, and/or 145-236 of the wild type BTN3A3 amino acid sequence set forth as SEQ ID NO: 6, or the corresponding region of another wild type BTN3A3 polypeptide).

In some cases, the BTN3A ectodomain of a BTN3A ectodomain polypeptide includes two immunoglobulin domains. For example, in some cases, the BTN3A ectodomain of a BTN3A ectodomain polypeptide includes (i) an amino acid sequence having 85% or more sequence identity (e.g., 90% or more, 95% or more, 98% or more, 99% or more, 99.2% or more, 99.5% or more, 99.8% or more, 99.9% or more, or 100% sequence identity) to a first immunoglobulin domain of an ectodomain of a wild type BTN3A protein (e.g., BTN3A1, BTN3A2, BTN3A3) (e.g., amino acids 26-145, 26-139, 30-143, 37-144, 36-145, 37-143, 45-144, and/or 30-139 of the wild type BTN3A1 amino acid sequence set forth as SEQ ID NO: 1, or to the corresponding region of another wild type BTN3A1 polypeptide; amino acids 26-145, 26-139, 30-143, 37-144, 36-145, 37-143, 45-144, and/or 30-139 of the wild type BTN3A2 amino acid sequence set forth as SEQ ID NO: 4, or the corresponding region of another wild type BTN3A2 polypeptide; amino acids 26-145, 26-139, 30-143, 37-144, 36-145, 37-143, 45-144, and/or 30-139 of the wild type BTN3A3 amino acid sequence set forth as SEQ ID NO: 6, or the corresponding region of another wild type BTN3A3 polypeptide); and also includes (ii) an amino acid sequence having 85% or more sequence identity (e.g., 90% or more, 95% or more, 98% or more, 99% or more, 99.2% or more, 99.5% or more, 99.8% or more, 99.9% or more, or 100% sequence identity) to a second immunoglobulin domain of an ectodomain of a wild type BTN3A protein (e.g., BTN3A1, BTN3A2, BTN3A3) (e.g., amino acids 145-236, 164-228, 164-236, 154-231, 161-186, and/or 145-236 of the wild type BTN3A1 amino acid sequence set forth as SEQ ID NO: 1, or to the corresponding region of another wild type BTN3A1 polypeptide; amino acids 157-228, 157-183, and/or 164-228 of the wild type BTN3A2 amino acid sequence set forth as SEQ ID NO: 4, or the corresponding region of another wild type BTN3A2 polypeptide; amino acids 164-236, 158-182, and/or 145-236 of the wild type BTN3A3 amino acid sequence set forth as SEQ ID NO: 6, or the corresponding region of another wild type BTN3A3 polypeptide).

In some cases, the BTN3A ectodomain includes a first domain from a wild type BTN3A protein and a second domain from a different wild type BTN3A protein. Thus, in some cases, a subject BTN3A ectodomain polypeptide is chimeric such that it has a first domain (e.g., an immunoglobulin domain such as an Immunoglobulin V-set domain; an Immunoglobulin (IG) domain, an IG_like domain, an Ig_MOG_like domain; and/or an Ig-like V-type 1 domain) from one BTN3A polypeptide and a second domain (e.g., an immunoglobulin domain such as an immunoglobulin-like domain, an CD80-like C2-set immunoglobulin domain, and/or an Ig-like V-type 2 domain) from a different BTN3A polypeptide. As an illustrative non-limiting example, a subject BTN3A ectodomain polypeptide can include an ectodomain having a first immunoglobulin domain (e.g., an Immunoglobulin V-set domain; an Immunoglobulin (IG) domain, an IG_like domain, an Ig_MOG_like domain; an Ig-like V-type 1 domain) from BTN3A1 and a second immunoglobulin domain (e.g., an immunoglobulin-like domain, an CD80-like C2-set immunoglobulin domain, an Ig-like V-type 2 domain) from BTN3A2. Any and all combinations are contemplated (e.g., BTN3A1/BTN3A2; BTN3A1/BTN3A3; BTN3A2/BTN3A1; BTN3A2/BTN3A3; BTN3A3/BTN3A1; BTN3A3/BTN3A2).

In some cases, the BTN3A ectodomain of a BTN3A ectodomain polypeptide includes an amino acid sequence having 85% or more sequence identity (e.g., 90% or more, 95% or more, 98% or more, 99% or more, 99.2% or more, 99.5% or more, 99.8% or more, 99.9% or more, or 100% sequence identity) to an immunoglobulin domain of a wild type BTN3A polypeptide (e.g., BTN3A1, BTN3A2, or BTN3A3) (e.g., an Immunoglobulin-like domain; an Immunoglobulin V-set domain; an Immunoglobulin (IG) domain, an IG_like domain, an Ig_MOG_like domain; an Ig-like V-type 1 domain, an CD80-like C2-set immunoglobulin domain, and Ig-like V-type 2 domain, and the like).

In some cases, the BTN3A ectodomain of a BTN3A ectodomain polypeptide includes two immunoglobulin domains. For example, in some cases, the BTN3A ectodomain of a BTN3A ectodomain polypeptide includes an amino acid sequence having 85% or more sequence identity (e.g., 90% or more, 95% or more, 98% or more, 99% or more, 99.2% or more, 99.5% or more, 99.8% or more, 99.9% or more, or 100% sequence identity) to a first immunoglobulin domain of a wild type BTN3A polypeptide (e.g., BTN3A1, BTN3A2, or BTN3A3) (e.g., an Immunoglobulin-like domain; an Immunoglobulin V-set domain; an Immunoglobulin (IG) domain, an IG_like domain, an Ig_MOG_like domain; an Ig-like V-type 1 domain, and the like) and also includes an amino acid sequence having 85% or more sequence identity (e.g., 90% or more, 95% or more, 98% or more, 99% or more, 99.2% or more, 99.5% or more, 99.8% or more, 99.9% or more, or 100% sequence identity) to a second immunoglobulin domain of a wild type BTN3A polypeptide (e.g., BTN3A1, BTN3A2, or BTN3A3) (e.g., an immunoglobulin-like domain, an CD80-like C2-set immunoglobulin domain, and Ig-like V-type 2 domain, and the like).

A BTN3A ectodomain polypeptide (e.g., a BTN3A ectodomain monomer, a BTN3A ectodomain dimer, a BTN3A ectodomain-Fc fusion) can include all or a portion of the immunoglobulin domains of the BTN3A ectodomain (e.g., BTN3A1 ectodomain, BTN3A2 ectodomain, BTN3A3 ectodomain,); and can further comprise one or more amino acids from a BTN3A polypeptide (e.g., BTN3A1, BTN3A2, BTN3A3) outside of the immunoglobulin domains; and can comprise amino acid sequences other than BTN3A sequences, which can include without limitation immunoglobulin Fc region sequences, sequences that confer binding to a target molecule other than the target molecule bound by the BTN3A ectodomain (e.g., other than LTβR, FLT1, HLA-E, CD163, and/or ROR2), dimerization sequences, signal sequences, detectable labels (e.g., a sequence that confers fluorescence, an affinity tag, and the like), etc. In some cases, the target molecule bound by the BTN3A ectodomain is selected from: LTβR, FLT1, HLA-E, CD163, and ROR2. In some cases, the target molecule bound by the BTN3A ectodomain is LTβR (also sometimes referred to in the art as LTBR, lymphotoxin beta receptor, TNFR superfamily member 3, CD18, TNFCR, TNFR3, TNFR-RP, TNFRSF3, TNFR2-RP, LT-BETA-R, and TNF-R-III). In some cases, the target molecule bound by the BTN3A ectodomain is FLT1 (also sometimes referred to in the art as fms-related tyrosine kinase 1, FLT, FLT-1, VEGF receptor 1, VEGFR1, and VEGFR-1). In some cases, the target molecule bound by the BTN3A ectodomain is HLA-E (also sometimes referred to in the art as a non-classical MHC molecule, major histocompatibility complex class I E, MHC, QA1, EA1.2, EA2.1, and HLA-6.2). In some cases, the target molecule bound by the BTN3A ectodomain is CD163 (also sometimes referred to in the art as a macrophage and γδ T cell scavenger receptor, M130, and MM130). In some cases, the target molecule bound by the BTN3A ectodomain is ROR2 (also sometimes referred to in the art as a Wnt receptor, receptor tyrosine kinase, receptor tyrosine kinase-like orphan receptor 2, BDB, BDB1, NTRKR2).

### Transmembrane Domain

The transmembrane domain of a wild type BTN3A (e.g., BTN3A1, BTN3A2, BTN3A3) is readily identifiable. As an illustrative example, the transmembrane domain as depicted on the NCBI reference sequence page was compared to the transmembrane domains as determined using three different prediction programs (TMHMM, TMpred, and TOPCONS) that were run on the wild type human BTN3A1, BTN3A2, and BTN3A3 amino acid sequences set forth in SEQ ID NOs: 1, 4, and 6, respectively.

The following overlapping amino acid regions of BTN3A1 were determined to define a transmembrane domain: 255-271, 249-271, 248-269, 248-271, and 248-266. Thus, a transmembrane domain is present at amino acids 248-271 (e.g., 255-271, 248-266, 248-269, 248-271, and/or 249-271) of the wild type human BTN3A1 protein set forth in SEQ ID NO: 1. Thus, in some cases, a BTN3A1 ectodomain lacks amino acids 248-271, 255-271, 248-266, 244-269, and/or 249-271 of the wild type human BTN3A1 protein set forth in SEQ ID NO: 1, or the corresponding region of another wild type BTN3A1 protein (e.g., another isoform, a BTN3A1 from a different species, etc.). In some cases, a subject BTN3A1 ectodomain polypeptide lacks a BTN3A1 transmembrane domain. In some cases, a BTN3A1 ectodomain polypeptide lacks a transmembrane domain (i.e., the BTN3A1 ectodomain polypeptide does not include a transmembrane domain, e.g., of any kind). In some cases, a subject BTN3A1 ectodomain polypeptide lacks a BTN3A1 transmembrane domain, but includes a heterologous transmembrane domain (i.e., a transmembrane domain form a protein other than BTN3A1). In some cases, a subject BTN3A1 ectodomain polypeptide includes a transmembrane domain (e.g., a heterologous transmembrane domain, a BTN3A1 transmembrane domain), and includes a cleavable linker between the BTN3A1 ectodomain and the transmembrane domain.

The transmembrane domain of a wild type BTN3A2 is readily identifiable. As an illustrative example, the transmembrane domain as depicted on the NCBI reference sequence page was compared to the transmembrane domains as determined using three different prediction programs (TMHMM, TMpred, and TOPCONS) that were run on the wild type human BTN3A2 set forth in SEQ ID NO: 4. The following overlapping amino acid regions were determined to define a transmembrane domain: 249-269, 248-270, 251-271, and 248-269. Thus, a transmembrane domain is present at amino acids 248-271 (e.g., 249-269, 248-270, 251-271, and/or 248-269) of the wild type human BTN3A2 protein set forth in SEQ ID NO: 4. Thus, in some cases, a BTN3A2 ectodomain lacks amino acids 248-271, 249-269, 248-270, 251-271, and/or 248-269 of the wild type human BTN3A2 protein set forth in SEQ ID NO: 4, or the corresponding region of another wild type BTN3A2 protein (e.g., another isoform, a BTN3A2 from a different species, etc.). In some cases, a subject BTN3A2 ectodomain polypeptide lacks a BTN3A2 transmembrane domain. In some cases, a BTN3A2 ectodomain polypeptide lacks a transmembrane domain (i.e., the BTN3A2 ectodomain polypeptide does not include a transmembrane domain, e.g., of any kind). In some cases, a subject BTN3A2 ectodomain polypeptide lacks a BTN3A2 transmembrane domain, but includes a heterologous transmembrane domain (i.e., a transmembrane domain form a protein other than BTN3A2). In some cases, a subject BTN3A2 ectodomain polypeptide includes a transmembrane domain (e.g., a heterologous transmembrane domain, a BTN3A2 transmembrane domain), and includes a cleavable linker between the BTN3A2 ectodomain and the transmembrane domain.

The transmembrane domain of a wild type BTN3A3 is readily identifiable. As an illustrative example, the transmembrane domain as depicted on the NCBI reference sequence page was compared to the transmembrane domains as determined using three different prediction programs (TMHMM, TMpred, and TOPCONS) that were run on the wild type human BTN3A3 set forth in SEQ ID NO: 6. The following overlapping amino acid regions were determined to define a transmembrane domain: 249-269, 248-270, 247-271, and 248-269. Thus, a transmembrane domain is present at amino acids 247-271 (e.g., 249-269, 248-270, and/or 248-269) of the wild type human BTN3A3 protein set forth in SEQ ID NO: 6. Thus, in some cases, a BTN3A3 ectodomain lacks amino acids 249-269, 248-270, 247-271, and/or 248-269 of the wild type human BTN3A3 protein set forth in SEQ ID NO: 6, or the corresponding region of another wild type BTN3A3 protein (e.g., another isoform, a BTN3A3 from a different species, etc.). In some cases, a subject BTN3A3 ectodomain polypeptide lacks a BTN3A3 transmembrane domain. In some cases, a BTN3A3 ectodomain polypeptide lacks a transmembrane domain (i.e., the BTN3A3 ectodomain polypeptide does not include a transmembrane domain, e.g., of any kind). In some cases, a subject BTN3A3 ectodomain polypeptide lacks a BTN3A3 transmembrane domain, but includes a heterologous transmembrane domain (i.e., a transmembrane domain form a protein other than BTN3A3). In some cases, a subject BTN3A3 ectodomain polypeptide includes a transmembrane domain (e.g., a heterologous transmembrane domain, a BTN3A3 transmembrane domain), and includes a cleavable linker between the BTN3A3 ectodomain and the transmembrane domain.

In some cases, a subject BTN3A ectodomain polypeptide lacks a transmembrane domain (i.e., does not include a transmembrane domain). Thus, in some embodiments, a subject BTN3A ectodomain polypeptide is soluble. In some cases, a subject BTN3A ectodomain polypeptide includes a transmembrane domain that is fused to a BTN3A ectodomain via a cleavable linker. Such a transmembrane domain can be removed from the BTN3A ectodomain polypeptide by cleavage of the linker. Any convenient cleavable linker can be used and many suitable cleavable linkers will be known to one of ordinary skill in the art.

### High Affinity BTN3A Ectodomain Polypeptide.

A “high affinity BTN3A ectodomain polypeptide” is a BTN3A ectodomain polypeptide (as defined above, and thus lacks a transmembrane domain of a wild type BTN3A protein) that has an amino acid mutation (i.e., an amino acid change) relative to a corresponding wild type BTN3A protein (e.g., BTN3A1, BTN3A2, BTN3A3) (e.g., relative to the corresponding region of a corresponding wild type BTN3A protein, relative to the ectodomain of a corresponding wild type BTN3A protein, relative to one or more immunoglobulin domains of a corresponding wild type BTN3A protein, relative to a native BTN3A ectodomain polypeptide, etc.), where the amino acid mutation increases the affinity of the BTN3A ectodomain polypeptide for its target molecule (binding partner) such that the affinity for the binding partner of the high affinity BTN3A ectodomain polypeptide is greater than the affinity for the binding partner of a corresponding wild type BTN3A protein (e.g., greater than the affinity for the binding partner of a corresponding ectodomain of a corresponding BTN3A polypeptide; greater than the affinity for the binding partner of a corresponding native BTN3A ectodomain polypeptide for the binding partner; greater than the affinity for the binding partner of the BTN3A ectodomain polypeptide prior to mutation; greater than the affinity for the binding partner of the corresponding unmutated BTN3A ectodomain polypeptide; etc.). For example, the amino acid mutation can increase the affinity by decreasing the off-rate by at least 10-fold, at least 20-fold, at least 50-fold, at least 100-fold, at least 500-fold, or more.

Binding can be determined by, for example, measuring the ability of an unlabeled BTN3A ectodomain polypeptide to compete with a labeled BTN3A ectodomain (e.g., a labeled native BTN3A ectodomain polypeptide, as defined above) for binding to a binding partner. Accordingly, relative biding can be assessed by comparing the results using a candidate unlabeled high-affinity BTN3A ectodomain polypeptide to results using an unlabeled native BTN3A ectodomain polypeptide (as defined above, a BTN3A ectodomain polypeptide that does not have an amino acid change relative to the corresponding sequence of a corresponding wild type BTN3A protein).

A high affinity BTN3A ectodomain polypeptide includes a BTN3A ectodomain (e.g., a BTN3A1 ectodomain, a BTN3A2 ectodomain, a BTN3A3 ectodomain) having an amino acid change (mutation) (e.g., 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 amino acid changes (mutations)) relative to a corresponding wild type BTN3A ectodomain (e.g., relative to the corresponding region of a corresponding wild type BTN3A polypeptide, e.g., a mammalian wild type BTN3A polypeptide such as the human wild type BTN3A protein set forth in any of SEQ ID NOs: 1, 4, and 6.).

In some cases, a high affinity BTN3A ectodomain polypeptide includes an amino acid change (mutation)(e.g., 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 amino acid changes (mutations)) relative to the immunoglobulin domain of a wild type BTN3A polypeptide (e.g, BTN3A1, BTN3A2, BTN3A3) (e.g., amino acids 26-145, 26-139, 30-143, 37-144, 36-145, 37-143, 45-144, 30-139, 145-236, 164-228, 164-236, 154-231, 161-186, and/or 145-236 of the wild type human BTN3A1 polypeptide amino acid sequence set forth in SEQ ID NO: 1, or the corresponding region of another wild type BTN3A1 protein, e.g., another mammalian wild type BTN3A1 protein; amino 26-145, 26-139, 30-143, 37-144, 36-145, 37-143, 45-144, 30-139, 157-228, 157-183, and/or 164-228 of the wild type human BTN3A2 polypeptide amino acid sequence set forth in SEQ ID NO: 4, or the corresponding region of another wild type BTN3A2 protein, e.g., another mammalian wild type BTN3A2 protein; amino acids 26-145, 26-139, 30-143, 37-144, 36-145, 37-143, 45-144, 30-139, 164-236, 158-182, and/or 145-236 of the wild type human BTN3A3 polypeptide amino acid sequence set forth in SEQ ID NO: 6, or the corresponding region of another wild type BTN3A3 protein, e.g., another mammalian wild type BTN3A3 protein).

In some cases, a high affinity BTN3A ectodomain polypeptide includes an amino acid change (e.g., 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 amino acid changes) relative to the ectodomain of a wild type BTN3A polypeptide.

In some cases, a high affinity BTN3A ectodomain polypeptide includes an amino acid change (e.g., 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 amino acid changes) relative to the ectodomain of a wild type BTN3A polypeptide, and the BTN3A ectodomain has an amino acid sequence having 85% or more sequence identity (e.g., 90% or more, 95% or more, 98% or more, 99% or more, 99.2% or more, 99.5% or more, 99.8% or more, 99.9% or more, or 100% sequence identity) to a corresponding BTN3A ectodomain (e.g, BTN3A1 ectodomain, BTN3A2 ectodomain, BTN3A3 ectodomain) (e.g., a polypeptide having (i) amino acids 26-145, 26-139, 30-143, 37-144, 36-145, 37-143, 45-144, and/or 30-139 and (ii) amino acids 145-236, 164-228, 164-236, 154-231, 161-186, and/or 145-236 of the wild type BTN3A1 amino acid sequence set forth as SEQ ID NO: 1; a polypeptide having (i) amino acids 26-145, 26-139, 30-143, 37-144, 36-145, 37-143, 45-144, and/or 30-139 and (ii) amino acids 157-228, 157-183, and/or 164-228 of the wild type BTN3A2 amino acid sequence set forth as SEQ ID NO: 4; a polypeptide having (i) amino acids 26-145, 26-139, 30-143, 37-144, 36-145, 37-143, 45-144, and/or 30-139 and (ii) amino acids 164-236, 158-182, and/or 145-236 of the wild type BTN3A3 amino acid sequence set forth as SEQ ID NO: 6).

In some cases, a BTN3A ectodomain polypeptide of the disclosure includes one or more (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 14 or more, 15 or more) amino acid changes relative to a corresponding BTN3A wild type protein (e.g., BTN3A1, BTN3A2, BTN3A3).

A BTN3A ectodomain polypeptide having no mutations relative to the corresponding region of a wild type BTN3A protein (i.e., where the BTN3A ectodomain polypeptide is a fragment of a wild type protein) is sometimes referred to herein as a “native BTN3A ectodomain polypeptide.” A native BTN3A ectodomain polypeptide can be used as a control in various instances, for example, in some cases when determining whether a BTN3A ectodomain polypeptide having one or more mutations relative to a wild type BTN3A ectodomain is a “high affinity BTN3A ectodomain polypeptide.”

According to the present disclosure, amino acid mutations (i.e., changes) include any naturally occurring or man-made amino acid modifications known or later discovered in the field. In some embodiments, amino acid changes include, e.g., substitution, deletion, addition, insertion, etc. of one or more amino acids. In some embodiments, amino acid changes include replacing an existing amino acid with another amino acid. In related embodiments, amino acid changes include replacing one or more existing amino acids with non-natural amino acids, or inserting one or more non-natural amino acids. Amino acid changes may be made in 1 or more (e.g, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, etc.) amino acid residues relative to a wild type sequence. The one or more amino acid changes can confer various properties to the high affinity BTN3A ectodomain polypeptide, e.g., affecting the stability, binding activity and/or specificity, etc.

Methods of generating and/or identifying a high affinity BTN3A ectodomain polypeptide are described below.

### Fusion Proteins

In some embodiments, a BTN3A ectodomain polypeptide includes a heterologous amino acid sequence (e.g., a region that specifically binds a target molecule other than the target molecule bound by the BTN3A ectodomain portion; a dimerization region, e.g., a protein-protein interaction domain such as a leucine zipper motif; an Fc region, and the like). In some cases, the target molecule bound by the BTN3A ectodomain is selected from: LTβR, FLT1, HLA-E, CD163, and ROR2. In some cases, the target molecule bound by the BTN3A ectodomain is LTβR. In some cases, the target molecule bound by the BTN3A ectodomain is FLT1. In some cases, the target molecule bound by the BTN3A ectodomain is HLA-E. In some cases, the target molecule bound by the BTN3A ectodomain is CD163. In some cases, the target molecule bound by the BTN3A ectodomain is ROR2. Thus, in some cases a BTN3A ectodomain polypeptide includes a heterologous amino acid sequence that specifically binds a target molecule other than LTβR, FLT1, HLA-E, CD163, or ROR2. In some cases a BTN3A ectodomain polypeptide includes a heterologous amino acid sequence that specifically binds a target molecule other than LTβR. In some cases a BTN3A ectodomain polypeptide includes a heterologous amino acid sequence that specifically binds a target molecule other than FLT1. In some cases a BTN3A ectodomain polypeptide includes a heterologous amino acid sequence that specifically binds a target molecule other than HLA-E. In some cases a BTN3A ectodomain polypeptide includes a heterologous amino acid sequence that specifically binds a target molecule other than CD163. In some cases a BTN3A ectodomain polypeptide includes a heterologous amino acid sequence that specifically binds a target molecule other than ROR2.

In some embodiments, a BTN3A ectodomain polypeptide of the present disclosure is a fusion protein, e.g., a BTN3A ectodomain fused in frame with a second polypeptide (a fusion partner). In some embodiments, the second polypeptide (the fusion partner) is capable of increasing the size of the fusion protein, e.g., so that the fusion protein will not be cleared from the circulation rapidly. In some cases, tissue penetration (i.e., the ability to penetrate tissues) can be a distinct advantage of using a subject BTN3A ectodomain polypeptide due to its relatively small size (e.g., compared to a much larger protein such as an antibody). In some cases, a BTN3A ectodomain of a BTN3A ectodomain polypeptide is not fused to a second polypeptide. In some cases, a fusion partner is a second polypeptide that is small enough so as not to limit the tissue penetration of the BTN3A ectodomain polypeptide to an unacceptable level (which would depend on the context of the particular method and/or desired outcome). Thus, in some cases, the second polypeptide (i.e., the polypeptide to which a subject BTN3A ectodomain is fused) is 200 amino acids or less (e.g., 190 amino acids or less, 180 amino acids or less, 170 amino acids or less, 160 amino acids or less, 150 amino acids or less, 140 amino acids or less, 130 amino acids or less, 120 amino acids or less, 110 amino acids or less, 100 amino acids or less, 90 amino acids or less, 80 amino acids or less, 70 amino acids or less, 60 amino acids or less, 50 amino acids or less, 40 amino acids or less, or 30 amino acids or less).

In some embodiments, the second polypeptide (the fusion partner for a BTN3A ectodomain of a subject BTN3A ectodomain polypeptide) is part or whole of an immunoglobulin Fc region (i.e., an antibody Fc sequence). In other embodiments, the second polypeptide is any suitable polypeptide that is substantially similar to Fc, e.g., providing increased size, multimerization domains, and/or additional binding or interaction with Ig molecules. These fusion proteins can facilitate purification, multimerization, and show an increased half-life in vivo. Fusion proteins having disulfide-linked multimeric structures can also, in some cases, be more efficient in binding and neutralizing other molecules than a monomeric BTN3A ectodomain polypeptide.

When fused to a heterologous polypeptide, the portion corresponding to the BTN3A ectodomain can be referred to as the “BTN3A ectodomain portion” of a subject BTN3A ectodomain polypeptide. In some cases, the BTN3A ectodomain (e.g., the BTN3A ectodomain portion) can be 100 amino acids or more in length (e.g., 110 amino acids or more, 125 amino acids or more, 150 amino acids or more, 90 amino acids or more, 95 amino acids or more, 100 amino acids or more, 105 amino acids or more, 110 amino acids or more, 115 amino acids or more, 120 amino acids or more, 125 amino acids or more, or 130 amino acids or more), up to the full-length of the portion of the wild-type protein that is N-terminal to the transmembrane domain (e.g., 247-254 amino acids for the human BTN3A1 protein), and can further be fused to a heterologous polypeptide, e.g. an immunoglobulin Fc.

In some cases, a BTN3A ectodomain polypeptide (e.g., the BTN3A ectodomain portion) has a length in a range of from 100 amino acids to 250 amino acids (e.g., from 100 amino acids to 225 amino acids, from 100 amino acids to 200 amino acids, from 100 amino acids to 175 amino acids, from 100 amino acids to 150 amino acids, from 150 amino acids to 250 amino acids, from 150 amino acids to 225 amino acids, from 150 amino acids to 200 amino acids, or from 150 amino acids to 175 amino acids).

In some embodiments, a BTN3A ectodomain polypeptide is fused or otherwise joined to an immunoglobulin sequence to form a chimeric protein. The immunoglobulin sequence can be an immunoglobulin constant domain(s). The immunoglobulin moiety in such chimeras may be obtained from any species, usually human, and includes IgG1, IgG2, IgG3 or IgG4 subtypes, IgA, IgE, IgD or IgM. Included in the constant regions of interest are human IgG4 constant regions with the amino acid substitution S241P (see, for example, Angal et al. (1993) Mol Immunol. 30(1):105-8. The immunoglobulin moiety may comprise one or more domains, e.g. CH1, CH2, CH3, etc.

Chimeras constructed from a sequence linked to an appropriate immunoglobulin constant domain sequence are known in the art. In such fusions, the encoded chimeric polypeptide may retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion or binding characteristics of the BTN3A ectodomain:immunoglobulin chimeras. In some embodiments, the BTN3A ectodomain:immunoglobulin chimeras are assembled as monomers, or hetero- or homo-multimers, and in some cases as dimers or tetramers.

Although the presence of an immunoglobulin light chain is not required, an immunoglobulin light chain may be included, either covalently associated to a BTN3A ectodomain:immunoglobulin heavy chain fusion polypeptide, or directly fused to the polypeptide. A single chain construct may be used to provide both heavy and light chain constant regions.

In other fusion protein constructs, the second polypeptide is a marker sequence (e.g., an affinity tag), such as a peptide that facilitates purification of the fused polypeptide. For example, the marker amino acid sequence can be a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824, 1989, for instance, hexa-histidine provides for convenient purification of the fusion protein. Another peptide tag useful for purification, the “HA” tag, corresponds to an epitope derived from the influenza hemagglutinin protein. Wilson et al., Cell 37: 767, 1984. The addition of peptide moieties to facilitate handling of polypeptides are familiar and routine techniques in the art.

In some embodiments, a BTN3A ectodomain polypeptide has a BTN3A ectodomain (e.g., a BTN3A1 ectodomain, a BTN3A2 ectodomain, a BTN3A3 ectodomain) and includes a fusion partner that is part or whole of an Fc region. In some cases, the Fc region is a human IgG4 Fc region.

A subject BTN3A ectodomain polypeptide can be modified, e.g., joined to a wide variety of other oligopeptides or proteins for a variety of purposes. For example, post-translationally modified, for example by prenylation, acetylation, amidation, carboxylation, glycosylation, PEGylation (covalent attachment of polyethylene glycol (PEG) polymer chains), etc. Such modifications can also include modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. In some embodiments, a subject BTN3A ectodomain polypeptide has one or more phosphorylated amino acid residues, e.g. phosphotyrosine, phosphoserine, or phosphothreonine.

In some other embodiments, BTN3A ectodomain polypeptides of the disclosure include reagents further modified to improve their resistance to proteolytic degradation or to optimize solubility properties or to render them more suitable as a therapeutic agent. For example, variants of the present disclosure further include analogs containing residues other than naturally occurring L-amino acids, e.g. D-amino acids or non-naturally occurring synthetic amino acids. D-amino acids may be substituted for some or all of the amino acid residues.

In addition to use as a treatment for various disorders and diseases, BTN3A ectodomain polypeptides are useful, for example, as an adjuvant to increase immune function, (e.g., when combined with a specific binding agent, e.g. an antibody, in some cases to a tumor cell specific antibody as defined herein)(e.g., by stimulating APC activity). BTN3A ectodomain polypeptides are also useful as imaging agents, e.g. when conjugated to a detectable label, which can be used for various purposes, e.g., as diagnostic reagents.

### Dimerization Moiety

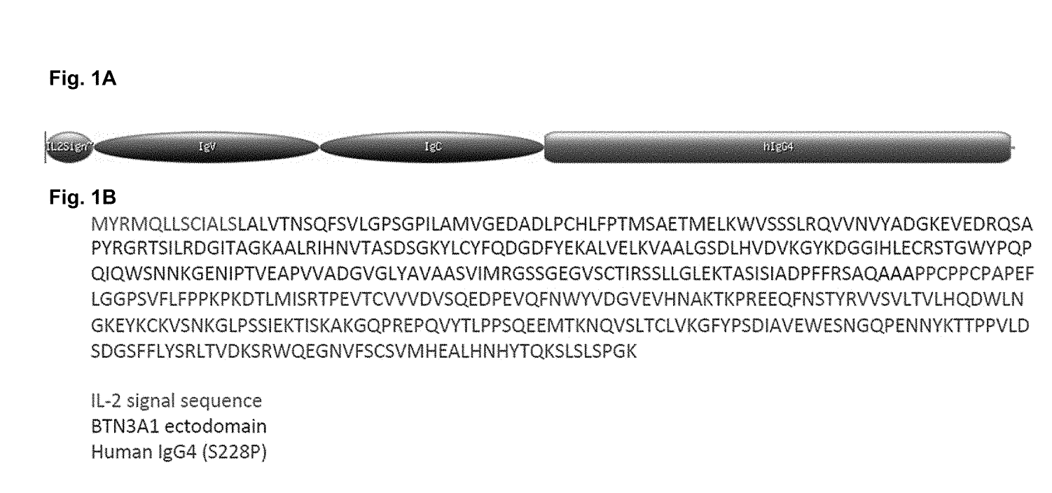

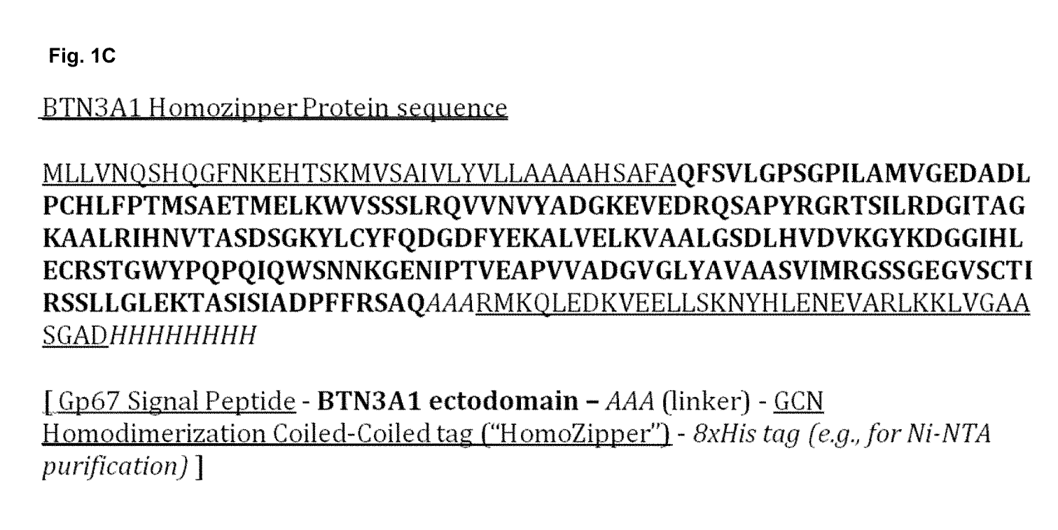

BTN3A ectodomain polypeptides can be monomeric or multimeric, i.e. dimer, trimer, tetramer, etc. For example, one or more BTN3A ectodomains can be covalently or non-covalently linked, e.g. as a fusion protein; disulfide bonded; through biotin binding to avidin, streptavidin; as a fusion protein where the fusion partner is a protein-protein interaction domain such as a leucine zipper motif, a CH3 domain from an antibody Fc region; etc. Such monomeric or multimeric (e.g. dimeric) BTN3A ectodomain polypeptides can be used as agents to stimulate an immune response (e.g., to stimulate a general immune response and/or to stimulate a response directed to a cell, e.g., a cancer cell expressing a target antigen, e.g., via co-administration with an opsonizing agent such as an ADCC-inducing antibody such as a monoclonal antibody). The term “dimerization moiety” as used herein is meant to encompass any moiety (including heterologous amino acid sequences that are present via fusion to a BTN3A ectodomain) that leads to dimerization of a subject BTN3A ectodomain polypeptide. In some embodiments, the second polypeptide (the fusion partner for a BTN3A ectodomain of a subject BTN3A ectodomain polypeptide) is a dimerization moiety. Thus, in some embodiments, a subject BTN3A ectodomain polypeptide includes a dimerization moiety. Examples of suitable dimeraization moieties include but are not limited to: a GCN Homodimerization Coiled-Coiled tag (see SEQ ID NO: 31 and FIG. 1C). The protein sequence of FIG. 1C (SEQ ID NO: 21) is an illustrative example of a BTNA ectodomain polypeptide having a dimerization moiety. Amino acids 39-255 of SEQ ID NO: 21 are an example of a BTNA ectodomain; amino acids 259-295 of SEQ ID NO: 21 (which amino acids are separately disclosed as SEQ ID NO: 31) are an example of a dimerization moiety; amino acids 1-38 of SEQ ID NO: 21 are an example of a signal sequence; and amino acids 296-303 of SEQ ID NO: 21 are an example of a His tag (an affinity tag).

Thus, in some cases, a subject BTN3A ectodomain polypeptide includes an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in SEQ ID NO: 31 (amino acids 259-295 of SEQ ID NO: 21). Thus, in some cases, a subject BTN3A ectodomain polypeptide includes a dimerization moiety that includes an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in SEQ ID NO: 31.

In some cases, a subject BTN3A ectodomain polypeptide is a BTN3A1 ectodomain polypeptide having a dimerization moiety and includes an amino acid sequence having 70% or more sequence identity (e.g., 80% or more, 85% or more, 90% or more, 95% or more, 98% or more, 99% or more, 99.5% or more, or 100% sequence identity) with amino acids 39-295 of the amino acid sequence set forth in SEQ ID NO: 21. In some cases, a subject BTN3A ectodomain polypeptide is a BTN3A1 ectodomain polypeptide having a dimerization moiety and includes an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in SEQ ID NO: 21.

Additional suitable examples of dimerization moieties include but are not limited to synzip polypeptides such as

SYNZIP14:
(SEQ ID NO: 32)
NDLDAYEREAEKLEKKNEVLRNRLAALENELATLRQEVASMKQELQS
SYNZIP17:
(SEQ ID NO: 33)
NEKEELKSKKAELRNRIEQLKQKREQLKQKIANLRKEIEAYK
SYNZIP18:
(SEQ ID NO: 34)
SIAATLENDLARLENENARLEKDIANLERDLAKLEREEAYF

Thus, in some cases, a subject BTN3A ectodomain polypeptide includes a dimerization moiety that includes an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in any of SEQ ID NOs: 31-34. In some cases, a subject BTN3A ectodomain polypeptide includes a dimerization moiety that includes an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in SEQ ID NO: 32. In some cases, a subject BTN3A ectodomain polypeptide includes a dimerization moiety that includes an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in SEQ ID NO: 33. In some cases, a subject BTN3A ectodomain polypeptide includes a dimerization moiety that includes an amino acid sequence having 70% or more sequence identity (e.g., 80% or more, 85% or more, 90% or more, 95% or more, 98% or more, 99% or more, 99.5% or more, or 100% sequence identity) with the amino acid sequence set forth in SEQ ID NO: 34.

### Detectable Labels

In some embodiments of the disclosure, the BTN3A ectodomain polypeptide includes a detectable label. Suitable detectable labels include directly detectable and indirectly detectable labels. Suitable detectable labels can be detected by any convenient method (e.g., spectroscopic, photochemical, biochemical, immunochemical, electrical, optical, chemical, or other means). Examples of suitable detectable labels include, but are not limited to: biotin (e.g., which can be indirectly detected using streptavidin), a fluorescent dye (a detectable label) (e.g., fluorescein, Texas red, rhodamine, green fluorescent protein, and the like), a radiolabel (e.g., ³H, ¹²⁵I, ³⁵S, ¹⁴O, or ³²P), an enzyme (indirectly detectable) (e.g., peroxidase, alkaline phosphatase, galactosidase, and others commonly used in an ELISA), a fluorescent protein (e.g., green fluorescent protein, red fluorescent protein, yellow fluorescent protein, and the like), a metal label, a colorimetric label, a binding pair member, and the like. Any binding pair member can be suitable for use as an indirectly detectable label moiety. In some cases, a subject BTN3A ectodomain polypeptide can be conjugated to (e.g., fused with) a fluorescent polypeptide (e.g., a fluorescent protein such as GFP, CFP, YFP, RFP, and the like).

In some embodiments of the disclosure, the BTN3A ectodomain polypeptide is coupled or conjugated to one or more imaging moieties, i.e. a detectable label. As used herein, “cytotoxic moiety” refers to a moiety that inhibits cell growth or promotes cell death when proximate to or absorbed by the cell. Suitable cytotoxic moieties in this regard include radioactive isotopes (radionuclides), chemotoxic agents such as differentiation inducers and small chemotoxic drugs, toxin proteins, and derivatives thereof.

As utilized herein, “imaging moiety”, or detectable label, refers to a moiety that can be utilized to increase contrast between a tumor and the surrounding healthy tissue in a visualization technique, e.g., radiography, positron-emission tomography (PET), magnetic resonance imaging (MRI), direct or indirect visual inspection. Thus, suitable imaging moieties include radiography moieties, e.g. heavy metals and radiation emitting moieties, positron emitting moieties, magnetic resonance contrast moieties, and optically visible moieties (e.g., fluorescent or visible-spectrum dyes, visible particles, etc. It will be appreciated by one of ordinary skill that some overlap exists between what is a therapeutic moiety and what is an imaging moiety.

In general, therapeutic or imaging agents can be conjugated to (included as part of) a BTN3A ectodomain polypeptide by any suitable technique, with appropriate consideration of the need for pharmacokinetic stability and reduced overall toxicity to the patient. A direct reaction between an agent and target molecule is possible when each possesses a functional group capable of reacting with the other. For example, a nucleophilic group, such as an amino or sulfhydryl group, may be capable of reacting with a carbonyl-containing group, such as an anhydride or an acid halide, or with an alkyl group containing a good leaving group (e.g., a halide). Alternatively, a suitable chemical linker group may be used. A linker group can function as a spacer in order to avoid interference with binding capabilities.

It will be evident to those skilled in the art that a variety of bifunctional or polyfunctional reagents, both homo- and hetero-functional (such as those described in the catalog of the Pierce Chemical Co., Rockford, Ill.), may be employed as a linker group. Coupling may be effected, for example, through amino groups, carboxyl groups, sulfhydryl groups or oxidized carbohydrate residues. There are numerous references describing such methodology. Alternatively a BTN3A ectodomain polypeptide is linked to the cytotoxic or imaging moiety by the use of a non-covalent binding pair, such as streptavidin/biotin, or avidin/biotin. In these embodiments, one member of the pair is covalently coupled to a BTN3A ectodomain polypeptide and the other member of the binding pair is covalently coupled to the cytotoxic or imaging moiety. It may be desirable to couple more than one cytotoxic and/or imaging moiety. By poly-derivatizing the BTN3A ectodomain polypeptide, several strategies may be simultaneously implemented, an antibody may be made useful as a contrasting agent for several visualization techniques, or a therapeutic antibody may be labeled for tracking by a visualization technique.

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear an agent by non-covalent associations, such as non-covalent bonding or by encapsulation

Carriers and linkers specific for radionuclide agents include radiohalogenated small molecules and chelating compounds. A radionuclide chelate may be formed from chelating compounds that include those containing nitrogen and sulfur atoms as the donor atoms for binding the metal, or metal oxide, radionuclide.

Radiographic moieties for use as imaging moieties in the present disclosure include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the disclosure. Such moieties may be conjugated to the BTN3A ectodomain polypeptide through an acceptable chemical linker or chelation carrier. Positron emitting moieties for use in the present disclosure include ¹⁸F, which can be easily conjugated by a fluorination reaction with the BTN3A ectodomain polypeptide.

Magnetic resonance contrast moieties can include chelates of chromium(III), manganese(II), iron(II), nickel(II), copper(II), praseodymium(III), neodymium(III), samarium(III) and ytterbium(III) ion. Because of their very strong magnetic moment, the gadolinium(III), terbium(III), dysprosium(III), holmium(III), erbium(III), and iron(III) ions.

Optically visible moieties for use as imaging moieties include fluorescent dyes, or visible-spectrum dyes, visible particles, and other visible labeling moieties. Fluorescent dyes such as fluorescein, coumarin, rhodamine, bodipy Texas red, and cyanine dyes, are useful when sufficient excitation energy can be provided to the site to be inspected visually. Endoscopic visualization procedures may be more compatible with the use of such labels. Acceptable dyes include FDA-approved food dyes and colors, which are non-toxic, although pharmaceutically acceptable dyes which have been approved for internal administration are preferred.

The effective amount of an imaging conjugate compositions to be given to a particular patient will depend on a variety of factors, several of which will be different from patient to patient. A competent clinician will be able to determine an effective amount to facilitate the visualization of a tumor. Dosage will depend on the treatment of the tumor, route of administration, the nature of the therapeutics, sensitivity of the tumor to the therapeutics, etc. Utilizing ordinary skill, the competent clinician will be able to optimize the dosage of a particular therapeutic or imaging composition in the course of routine clinical trials.

A typical dose may be from 0.001 to 100 milligrams of conjugate per kilogram subject body weight. Relatively large doses, in the range of 0.1 to 10 mg per kilogram of patient body weight may be used for imaging conjugates with a relatively non-toxic imaging moiety. The amount utilized will depend on the sensitivity of the imaging method, and the relative toxicity of the imaging moiety.

### Multispecific BTN3A Ectodomain Polypeptides

In some embodiments, the second polypeptide (the fusion partner for a BTN3A ectodomain of a subject BTN3A ectodomain polypeptide) specifically binds to a target molecule other than the target molecule bound by the BTN3A ectodomain portion of the BTN3A polypeptide (e.g., other than LTβR, FLT1, HLA-E, CD163, and/or ROR2). Thus, in some embodiments, a subject BTN3A ectodomain polypeptide is multispecific (e.g., bispecific). The terms “multispecific” or “bispecific” are commonly used when referring to antibodies that recognize two or more different antigens by virtue of possessing at least one region (e.g., derived from a variable region of a first antibody) that is specific for a first antigen, and at least a second region (e.g., derived from a variable region of a second antibody) that is specific for a second antigen (These antibodies are also known as bifunctional antibodies or multifunctional antibodies). A bispecific antibody specifically binds to two target antigens and is thus one type of multispecific antibody.

In some embodiments, a subject BTN3A ectodomain polypeptide is multispecific (e.g., bispecific), such that a first region of the polypeptide corresponds to a subject BTN3A ectodomain polypeptide sequence (i.e., the first region includes a BTN3A ectodomain), and a second region that specifically binds to another target molecule (e.g., antigen). For example, in some cases, a BTN3A ectodomain is fused to a second polypeptide that binds specifically to a target molecule other than the target molecule bound by the BTN3A ectodomain. In some cases, the target molecule bound by the BTN3A ectodomain is selected from: LTβR, FLT1, HLA-E, CD163, and ROR2. In some cases, the target molecule bound by the BTN3A ectodomain is LTβR. In some cases, the target molecule bound by the BTN3A ectodomain is FLT1. In some cases, the target molecule bound by the BTN3A ectodomain is HLA-E. In some cases, the target molecule bound by the BTN3A ectodomain is CD163. In some cases, the target molecule bound by the BTN3A ectodomain is ROR2. Thus, in some cases a BTN3A ectodomain is fused to a second polypeptide that binds specifically to a target molecule other than LTβR, FLT1, HLA-E, CD163, or ROR2. In some cases a BTN3A ectodomain is fused to a second polypeptide that binds specifically to a target molecule other than LTβR. In some cases a BTN3A ectodomain is fused to a second polypeptide that binds specifically to a target molecule other than FLT1. In some cases a BTN3A ectodomain is fused to a second polypeptide that binds specifically to a target molecule other than HLA-E. In some cases a BTN3A ectodomain is fused to a second polypeptide that binds specifically to a target molecule other than CD163. In some cases a BTN3A ectodomain is fused to a second polypeptide that binds specifically to a target molecule other than ROR2.

In some cases, the second region includes an antibody derived sequence (e.g., a binding region of an antibody, e.g., comprising the CDRs of the antibody). In some cases, the second region includes binding sequences from antibodies that specifically bind tumor antigens. Tumor antigens of interest include, but are not limited to CD20, CD52, CD38, HER-2, 17-1A, and EGFR. Examples of antibodies with CDRs that provide specific binding to a cancer cell marker include, but are not limited to: CETUXIMAB (binds EGFR), PANITUMUMAB (binds EGFR), RITUXIMAB (binds CD20), TRASTUZUMAB (binds HER2), PERTUZUMAB (binds HER2), ALEMTUZUMAB (binds CD52), BRENTUXIMAB (binds CD30), and the like. In some cases, the second region includes binding sequences from an antibody that specifically binds an antigen selected from: CD47, CD19, CD20, CD22, CD24, CD25, CD30, CD33, CD38, CD44, CD52, CD56, CD70, CD96, CD97, CD99, CD123, CD279 (PD-1), PD-1L, EGFR, HER2, CD117, C-Met, PTHR2, and HAVCR2 (TIM3).

In some cases, the second region of a multispecific BTN3A polypeptide includes an ectodomain of a protein other than a BTN3A protein. Examples of proteins from which such an ectodomain can be derived include, but are not limited to: PD-1, PD-L1, CD47, and SIRPα. (e.g., a high affinity SIRPα variant/polypeptide).

In some cases, the second region of a multispecific BTN3A ectodomain polypeptide specifically binds an antigen selected from: CTLA-4, Lag-3, BTLA, Tim-3, CD244, CD40, CD40L, CD47, SIRPα, PD-1, and PD-L1. As such, in some cases, a subject BTN3A ectodomain polypeptide includes a fusion partner for the BTN3A ectodomain, where the fusion partner includes a region that specifically binds an antigen selected from: CTLA-4, Lag-3, BTLA, Tim-3, CD244, CD40, CD40L, CD47, SIRPα, PD-1, and PD-L1.

In some embodiments, a subject BTN3A ectodomain polypeptide includes a linker (e.g., a linker polypeptide). For example, in some embodiments, a subject BTN3A ectodomain polypeptide and a fusion partner are separated by a linker (e.g., a linker polypeptide). A linker polypeptide may have any of a variety of amino acid sequences. Proteins can be joined by a linker polypeptide can be of a flexible nature (e.g., a flexible linker polypeptide), although other chemical linkages are not excluded. Suitable linkers include polypeptides of between about 6 amino acids and about 40 amino acids in length, or between about 6 amino acids and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that the in some case, linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)ₙ, glycine-serine polymers (including, for example, (GS)ₙ, GSGGSₙ (SEQ ID NO: 35), GGSGGSₙ (SEQ ID NO: 36), and GGGSₙ (SEQ ID NO: 37), where n is an integer of at least one (e.g., where n is an integer of one, two, three, four, five, six, seven, eight, nine, ten, or greater than ten), glycine-alanine polymers, alanine-serine polymers. Exemplary linkers can comprise amino acid sequences including, but not limited to, GGSG (SEQ ID NO: 38), GGSGG (SEQ ID NO: 39), GSGSG (SEQ ID NO: 40), GSGGG (SEQ ID NO: 41), GGGSG (SEQ ID NO: 42), GSSSG (SEQ ID NO: 43), and the like. The ordinarily skilled artisan will recognize that design of a peptide conjugated to any elements described above can include linkers that are all or partially flexible, such that the linker can include a flexible linker as well as one or more portions that confer less flexible structure.

### Producing a BTN3A Ectodomain Polypeptide

BTN3A ectodomain polypeptides of the present disclosure can be produced by any suitable means known or later discovered in the field, e.g., produced from eukaryotic or prokaryotic cells, synthesized in vitro, etc. Where the protein is produced by prokaryotic cells, it may be further processed by unfolding, e.g. heat denaturation, DTT reduction, etc. and may be further refolded, using methods known in the art.

The polypeptides may be prepared by cell-free translation systems, or synthetic in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA encoding the polypeptides of interest may be chemically synthesized or transcribed in vitro. One of skill in the art can readily utilize well-known codon usage tables and synthetic methods to provide a suitable coding sequence for any of the polypeptides of the disclosure. The nucleic acids may be isolated and obtained in substantial purity. The nucleic acids, either as DNA or RNA, can be obtained substantially free of other nucleic acid sequences, generally being at least about 50%, usually at least about 90% pure. Subject nucleic acids can be “recombinant,” e.g., flanked by one or more nucleotides with which it is not normally associated on a naturally occurring chromosome. The nucleic acids of the disclosure can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the disclosure can be introduced into suitable host cells using a variety of techniques available in the art.

### Nucleic Acids and Production of a BTN3A Polypeptide

Compositions are provided that include a nucleic acid (e.g., RNA or DNA) encoding a subject BTN3A ectodomain polypeptide (i.e., a nucleic acid that includes a nucleotide sequence that encodes a subject BTN3A ectodomain polypeptide). The sequence encoding a subject BTN3A ectodomain polypeptide can be operably linked to a promoter operable in a desired cell type (e.g., a prokaryotic cell, a eukaryotic cell, a eukaryotic cell of a particular tissue type, a mammalian cell, a human cell, etc.).

The disclosure also provides isolated nucleic acids encoding a subject BTN3A ectodomain polypeptide, vectors and host cells comprising the nucleic acid, and recombinant techniques for the production of BTN3A ectodomain polypeptides.

For recombinant production of a subject BTN3A ectodomain polypeptide, a nucleic acid encoding the BTN3A ectodomain polypeptide can be inserted into a replicable vector for further cloning (amplification of the DNA) or for expression. DNA encoding a subject BTN3A ectodomain polypeptide can be readily isolated and sequenced using conventional procedures. Many vectors are available. The vector components can include, but are not limited to, one or more of the following: a signal sequence (i.e., a nucleotide sequence encoding a signal sequence that will be fused in frame with the BTN3A ectodomain of the BTN3A ectodomain polypeptide, which provides for secretion of the BTN3A ectodomain polypeptide), an origin of replication, one or more marker genes, an enhancer element, a promoter, and a transcription termination sequence.

A subject BTN3A ectodomain polypeptide of this disclosure may be produced recombinantly not only directly, but also as a fusion polypeptide with a heterologous polypeptide, which can include a signal sequence or other polypeptide having a specific cleavage site at the N-terminus of the mature protein or polypeptide. Thus, a BTN3A ectodomain polypeptide can include a signal sequence, which is generally cleaved away from the protein during secretion from a cell. A signal sequence can be any polypeptide (amino acid sequence) that is recognized and processed (i.e., cleaved by a signal peptidase) by the host cell. For example, a signal sequence can be the BTN3A signal sequence or can be a heterologous signal sequence (e.g., a Gp67 signal peptide (SEQ ID NO: 26), a IL-2 signal sequence (SEQ ID NO: 27), etc.). For prokaryotic host cells that do not recognize and process a native eukaryotic signal sequence, the signal sequence can be substituted by a prokaryotic signal sequence.

An “isolated” nucleic acid molecule is a nucleic acid molecule that is identified and separated from at least one contaminant nucleic acid molecule with which it is ordinarily associated prior to isolation. An isolated nucleic acid molecule is other than in the form or setting in which it can be found in nature. Isolated nucleic acid molecules therefore are distinguished from the nucleic acid molecule as it exists in natural cells. In the present disclosure, a BTN3A ectodomain polypeptide by definition is not naturally occurring in that it does not include a BTN3A transmembrane domain.

Examples of suitable host cells for cloning or expressing subject nucleic acids include, but are not limited to prokaryote, yeast, or higher eukaryote cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1.982)); MRC 5 cells; FS4 cells; and a human hepatoma line (Hep G2). Host cells can be transformed with the above-described expression or cloning vectors for BTN3A ectodomain polypeptide production and cultured in conventional nutrient media modified as appropriate for inducing promoters, selecting transformants, or amplifying the genes encoding the desired sequences.

### Introduction of Nucleic Acids

In some cases, a subject BTN3A ectodomain polypeptide is administered to an individual (and/or introduced into a cell) by providing the BTN3A ectodomain polypeptide as a nucleic acid (e.g., an RNA, e.g., an mRNA; or a DNA, e.g., a recombinant expression vector, a linear DNA, a circular DNA, a plasmid, a viral vector, etc.) encoding the BTN3A ectodomain polypeptide. This disclosure provides such methods and also the nucleic acids for such methods.

For example, an mRNA encoding a subject BTN3A ectodomain polypeptide can be introduced into a cell, and the cell can then secret the translated protein. As another example, a DNA (e.g., a recombinant expression vector, a linear DNA, a circular DNA, a plasmid, a viral vector, etc.) encoding a subject BTN3A ectodomain polypeptide can be introduced into a cell and the cell can then produce and secret the encoded protein. Therefore, in some cases, a nucleic acid encoding a subject BTN3A ectodomain polypeptide includes a nucleotide sequence encoding a signal sequence (e.g., upstream of and in frame with the nucleotide sequence that encodes the BTN3A ectodomain polypeptide). As would be readily recognized by one of ordinary skill in the art, a signal sequence as referred to here is an amino acid sequence at or near the amino terminus of a nascent protein that can be recognized by the signal recognition particle of a eukaryotic cell, resulting in transport of the protein into the secretory pathway of the cell, thus facilitating secretion of a protein from the cell (e.g., the signal sequence can be cleaved from the protein). Any convenient signal sequence can be used.

In some cases, a nucleic acid encoding a subject BTN3A ectodomain polypeptide is introduced into a cell (e.g., in vivo, ex vivo, in vitro) and the cell can then produce and secret the encoded protein. In some cases, the cell is in vitro. In some cases, the cell is ex vivo. In some cases, the cell is in vivo (e.g., in some cases, a nucleic acid encoding a subject BTN3A ectodomain polypeptide is administered to an individual, e.g., systemically, locally, injected, injected intratumorally, injected locally, etc.). For example, in some cases, a nucleic acid encoding a BTN3A ectodomain polypeptide is introduced into a cell that is in vivo (e.g., in some cases, a nucleic acid encoding a BTN3A ectodomain polypeptide is introduced into a cell in vivo by administering the nucleic acid to an individual). In some cases, a nucleic acid encoding a subject BTN3A ectodomain polypeptide is introduced into a cell (e.g., ex vivo, in vitro) and the cell is then introduced into an individual. In some cases, the cell is autologous to the individual (e.g., the cell was isolated from the individual or is the progeny of a cell that was isolated from the individual).

In some cases (e.g., in any of the above scenarios, e.g., in vitro, ex vivo, in vivo), the cell into which a nucleic acid encoding a subject BTN3A ectodomain polypeptide is introduced is an immune cell (e.g., a leukocyte, a T cell, a CD8 T cell, a CD4 T cell, a memory/effector T cell, a B cell, a myeloid cell, an antigen presenting cell (APC), a dendritic cell, a macrophage, a monocyte, an NK cell, and the like). In some cases (e.g., in any of the above scenarios, e.g., in vitro, ex vivo, in vivo), the cell into which a nucleic acid encoding a subject BTN3A ectodomain polypeptide is introduced is a stem cell (e.g., a hematopoietic stem cell, a pluripotent stem cell, a multipotent stem cell, a tissue restricted stem cell, a self-renewing T cell, a long term memory T cell, etc.). In some cases (e.g., in any of the above scenarios, e.g., in vitro, ex vivo, in vivo), the cell into which a nucleic acid encoding a subject BTN3A ectodomain polypeptide is introduced is an immune cell (e.g., a lymphocyte, a leukocyte, a T cell, a CD8 T cell, a CD4 T cell, a regulatory T cell, a memory T cell, an effector T cell, a memory/effector T cell, a B cell, an antigen presenting cell (APC), a dendritic cell, a macrophage, a monocyte, an NK cell, and the like) or a stem cell (e.g., a hematopoietic stem cell, a pluripotent stem cell, a multipotent stem cell, a tissue restricted stem cell, a self-renewing T cell, a long term memory T cell, etc.). In some cases (e.g., in any of the above scenarios, e.g., in vitro, ex vivo, in vivo), the cell into which a nucleic acid encoding a subject BTN3A ectodomain polypeptide is introduced is a cancer cell (e.g., a subject nucleic acid can be introduced into a tumor, i.e., into a cell of a tumor).

In some cases (e.g., in any of the above scenarios, e.g., in vitro, ex vivo, in vivo), the cell into which a nucleic acid encoding a subject BTN3A ectodomain polypeptide is introduced is a T cell with an engineered T cell receptor (TCR) (such a cell is also referred to herein as a “TCR-engineered T cell”). As used herein the term “TCR-engineered T cell” refers to any T-cell having a T cell receptor that is heterologous to the T cell. Suitable examples include, but are not limited to (i) a T cell that includes a chimeric antigen receptor (CAR) (such a cell is also referred to herein as a “CAR-T cell” or an “engineered CAR-T cell”); and (ii) a T cell that includes a heterologous TCR that binds to an antigen such as a cancer antigen, e.g., MART1, NY-ESO-1, p53, and the like (e.g., such a cell can include a nucleic acid encoding the TcR-alpha and TcR-beta polypeptides of a heterologous TCR, such as a TCR that binds to an antigen such as a cancer antigen, e.g., MART1, NY-ESO-1, p53, and the like). In some cases, a T cell that includes a chimeric antigen receptor (CAR) is an ‘armored CAR T cell (e.g., a CAR-containing T cell that secretes one or more cytokines).

In some cases, a suitable TCR-engineered T cell can have an engineered TCR (e.g., a CAR, a heterologous TCR that binds to an antigen, etc.) that binds to a cancer marker (e.g., CD19, CD20, CD22, CD24, CD25, CD30, CD33, CD38, CD44, CD52, CD56, CD70, CD96, CD97, CD99, CD123, CD279 (PD-1), ROR1, EGFR, HER2, CD117, C-Met, PTHR2, and/or HAVCR2 (TIM3)). In some cases, a suitable TCR-engineered T cell can have an engineered TCR (e.g., a CAR, a heterologous TCR that binds to an antigen, etc.) that binds to a target antigen (e.g., any desired target antigen).

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” or “expression vector” and similar terms of the art are used interchangeably herein to refer to a DNA molecule comprising a vector and at least one insert. Recombinant expression vectors can be generated for the purpose of expressing and/or propagating the insert(s), or for the construction of other recombinant nucleotide sequences. The insert(s) (e.g., a nucleotide sequence encoding a subject BTN3A ectodomain polypeptide) may or may not be operably linked to a promoter sequence and may or may not be operably linked to DNA regulatory sequences. Thus in some cases, a nucleotide sequence encoding a subject BTN3A ectodomain polypeptide is operably linked to a promoter (e.g., one that is operable in a desired cell type, e.g., a eukaryotic cell, a mammalian cell, a primate cell, a human cell, an immune cell, a leukocyte, a T cell, a CD8 T cell, a CD4 T cell, a memory/effector T cell, a B cell, an antigen presenting cell (APC), a dendritic cell, a macrophage, a monocyte, an NK cell, a stem cell, a hematopoietic stem cell, a pluripotent stem cell, a multipotent stem cell, a tissue restricted stem cell, etc.).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a nucleic acid encoding a subject site-directed modifying polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle in mice).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase II-alpha (CamKlla) promoter (see, e.g., Mayford et al. (1996) Proc. Natl. Acad. Sci. USA 93:13250; and Casanova et al. (2001) Genesis 31:37); a CMV enhancer/platelet-derived growth factor-β promoter (see, e.g., Liu et al. (2004) Gene Therapy 11:52-60); and the like.

Adipocyte-specific spatially restricted promoters include, but are not limited to aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (see, e.g., Akyurek et al. (2000) Mol. Med. 6:983; and U.S. Pat. No. 7,169,874); a smoothelin promoter (see, e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csnl polypeptide) and/or regulate translation of an encoded polypeptide.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Also provided in this disclosure are cells that include a nucleic acid (e.g., as described above) that includes a nucleotide sequence encoding a subject BTN3A ectodomain polypeptide. Such a cell can be a cell from any organism (e.g., a bacterial cell, an archaeal cell, a cell of a single-cell eukaryotic organism, a plant cell, an algal cell, a fungal cell (e.g., a yeast cell), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal, a cell from a rodent, a cell from a human, etc.).

### Pharmaceutical Compositions

According to the present disclosure, BTN3A ectodomain polypeptides (and/or a nucleic acid encoding the same) can be provided in pharmaceutical compositions (pharmaceutical formulations) suitable for therapeutic use, e.g. for human treatment. In some embodiments, pharmaceutical compositions of the present disclosure include one or more therapeutic entities of the present disclosure or pharmaceutically acceptable salts, esters or solvates thereof. In some other embodiments, pharmaceutical compositions of the present disclosure include one or more therapeutic entities of the present disclosure in combination with another therapeutic agent, e.g., an anti-tumor agent.

Therapeutic entities of the present disclosure (e.g., a subject BTN3A ectodomain polypeptide) are often administered as pharmaceutical compositions (pharmaceutical formulations) comprising an active therapeutic agent (e.g., a subject BTN3A ectodomain polypeptide) and a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some cases, a subject composition (e.g., a therapeutic composition) consists of a BTN3A ectodomain polypeptide. In some cases, a subject composition (e.g., a therapeutic composition) consists essentially of a BTN3A ectodomain polypeptide. In some cases, a subject composition (e.g., a therapeutic composition) consists of a BTN3A ectodomain polypeptide and a pharmaceutically acceptable excipient. In some cases, a subject composition (e.g., a therapeutic composition) consists essentially of a BTN3A ectodomain polypeptide and a pharmaceutically acceptable excipient.

In still some other embodiments, pharmaceutical compositions of the present disclosure can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

### Methods of Use

Aspects of the disclosure include methods and compositions for inducing an immune response in an individual. For example, aspects of the disclosure include methods and compositions for activating an APC. Because such methods can be used to treat an individual, such methods can also be referred to as methods of treating an individual. Methods are provided for treating, reducing and/or or preventing cancer; treating, reducing and/or or preventing infection (e.g., chronic infection); and/or for treating, reducing and/or or preventing an immunological disease or disorder (e.g., an inflammatory disease, a condition associated with immunosuppression, etc.)(e.g., multiple sclerosis, arthritis, and the like). For example, in some cases, a subject BTN3A ectodomain polypeptide can be used as an immune stimulant (e.g., used for immunopotentiation). In some cases, a subject method is a method of treating an individual having cancer and/or having a chronic infection. In some cases, a subject method is a method of treating an individual who is immunosuppressed (e.g., an individual having primary or combined immunodeficiency) (e.g., an individual having Bruton's Agammaglobulinemia, an individual having Common Variable Immunodeficiency, an individual having X linked SCJD, and the like).

In general, the methods can include contacting an APC and/or a monocyte with a BTN3A ectodomain polypeptide in an amount and for a period of time effect to activate the APC or to induce the monocyte to differentiate and mature into an activated APC. Any of the BTN3A ectodomain polypeptides described herein (e.g., those having a fusion partner, e.g., a dimerization moiety, a signal sequence, an Fc region, a human IgG4 Fc, an affinity tag, etc.; monomeric forms; dimeric forms; multispecific forms; and the like) can be suitable for such methods. In some cases (e.g., when the method includes administering a subject BTN3A ectodomain polypeptide to an individual) an endogenous APC (an APC present in the individual) is contacted in vivo with the administered composition. Thus, the method can be considered an in vivo method of treating an individual. For example, a subject composition can be administered to an individual (e.g., systemically or locally, e.g., injected into or near a tumor, into or near a site of tumor resection, and the like) and endogenous APCs are thereby contacted with the BTN3A ectodomain polypeptide. The activated APCs can then contact endogenous naive T cells in vivo. As noted above, methods of treating, contacting, etc. can be performed by introducing a nucleic acid encoding a subject BTN3A ectodomain polypeptide into a cell (e.g., administering such a nucleic acid to an individual, introducing such a nucleic into a cell and then introducing/administering the cell into an individual, etc.).

In some cases, methods of treating can include a step of administering to the individual a BTN3A ectodomain polypeptide (e.g., in an amount (e.g., in a unit dose formulation) that is effective to treat the individual, e.g., reduce the number of cancer cells, reduce the number of infected cells, increase the number of activated APCs, increase the number of activated T cells, increase activity level of the immune system, and the like). In some cases, the method includes a step of measuring whether APCs of the individual were activated (e.g., see the assays and molecules to measure described above).

In some embodiments, subject methods include a step of obtaining or isolating an APC from an individual (e.g., isolating enriched populations of APCs, e.g., dendritic cells (DCs)). Techniques for the isolation, generation, and culture of APCs will be known to one of ordinary skill in the art and any convenient technique can be used. In some cases, the APCs are autologous to an individual who is being treated (i.e., are cells isolated from the individual or are cells derived from cells of the individual).

In some cases, a method of treating includes a step of introducing an activated APC (e.g., a population of activated APCs) into an individual. In some cases, a method of treating includes a step of contacting an activated APC with a naive T-cell to generate a (cross-primed) antigen specific effector cell. Such a step can take place in vitro or in vivo. In some cases, a method of treatment includes introducing into an individual the cross-primed antigen specific effector T cell. In some cases, the method includes a step of measuring whether APCs of the individual were activated (e.g., see the assays and molecules to measure described above) and/or a step of measuring whether naive T-cells were cross-primed into antigen specific effector T cells.

In some embodiments, an APC, (e.g., dendritic cell (DC), B cell, macrophage, and the like) and/or a monocyte is contacted with a subject BTN3A ectodomain polypeptide in vivo (e.g., the method includes administering the BTN3A ectodomain polypeptide to an individual). In some embodiments, an APC, (e.g., dendritic cell (DC), B cell, macrophage, and the like) and/or a monocyte is contacted with a subject BTN3A ectodomain polypeptide in vitro and/or ex vivo. The APC and/or monocyte can be from any source (e.g., can be from an in vitro cell culture, e.g., an established cell line; can be isolated from an individual, e.g., can be a primary cell; etc.). In some such cases, the APC and/or monocyte is isolated from an individual or derived from cells of the individual (e.g., APCs can be derived from isolated monocytes of the individual). In some cases, a subject method (e.g., any of the methods described herein) includes a step of measuring whether an activated APC was generated (e.g., whether contacting the APC and/or monocyte with a resulted in an activated APC, e.g., a population of activated APCs). Assays and molecules to measure whether an APC is activated are described above.

After an APC and/or monocyte is activated (e.g., via contact with a subject BTN3A ectodomain polypeptide in vitro or ex vivo), a resulting activated APC (e.g., a population of activated APCs) can be introduced into an individual (e.g., as a method of treatment). In cases where the APC and/or monocyte was obtained/isolated from the same individual or derived from cells of the same individual (e.g., APCs can be derived from isolated monocytes of the individual) into which the activated APC(s) are introduced, the cells can be considered autologous to the individual. In some cases, the activated APC (e.g., population of activated APCs) is not autologous to the individual (e.g., the APC and/or monocyte that was contacted with the BTN3A ectodomain polypeptide was not obtained/isolated from the same individual).

### Loading an APC

In some cases, a subject method includes a step of loading an APC. For example, an APC and/or monocyte can be contacted with a target antigen prior to, simultaneous with, or after contact with a subject BTN3A ectodomain polypeptide (e.g., to load the APC prior to contact with a T cell). In some embodiments, an APC and/or monocyte is contacted with a target antigen and a subject BTN3A ectodomain polypeptide at a dose and for a period of time effective for the uptake of the target antigen by the APC, thereby producing a loaded APC. In some cases, the APC and/or monocyte is contacted with the BTN3A ectodomain polypeptide prior to contact with a target antigen (and thus the target antigen is contacted by an activated APC). In some cases, the APC and/or monocyte is contacted with the BTN3A ectodomain polypeptide in the presence of (i.e., simultaneous with) a target antigen. In some cases, the APC and/or monocyte is contacted with the BTN3A ectodomain polypeptide after the APC has been contacted with a target antigen (e.g., contacted in vitro or ex vivo with a target antigen, e.g., a tumor cell lysate).

A target antigen can be any antigen which will be taken up by the APC. If the antigen is a protein, the APC will process it and subsequently present certain peptide components to T cells. In some cases, a target antigen can be a polypeptide, a protein complex, a mixture of polypeptides, and the like. In some cases, the target antigen is a cell (e.g., a cell from an individual). For example, in some cases, contacting the APC, comprises contacting an autologous APC with a cell (e.g., a cancer cell from the individual, e.g., a cell or cells from a tumor). In some cases, a target antigen is present in a complex mixture (e.g., a cellular lysate, a collection of plasma membrane proteins, etc, e.g., a lysate from a tumor). Thus, in some embodiments, a target antigen is present in a cellular lystate. In some such cases, a subject method can include contacting an APC with a lysate from cancer cells of the individual (i.e., a cancer cell cellular lysate, a lysate enriched for plasma membrane proteins, a lysate containing plasma membrane proteins, etc.). Cancer cells of the individual, which can be the source of the target antigen (e.g., the source of a cellular lysate) or can be the target antigen, can be any cancer cell of the individual (e.g., cells from primary and/or metastatic tumors; cancerous cells from the blood; lymph node cells; cells from pleural effusions (e.g., malignant pleural effusions), e.g., from a patient with lung cancer; cells from peritoneal effusions (e.g., malignant peritoneal effusions), e.g., from a patient with ovarian cancer; the involved skin of patients with mycosis fungoides; etc.).

Target antigens can be tumor specific or tumor associated antigens (e.g., whole tumor or cancer cells, a tumor cell lysate, tumor cell membrane preparations (e.g., a membrane fraction), tumor cell plasma membrane preparations (e.g., a plasma membrane fraction), isolated or partially isolated antigens from tumors, fusion proteins, liposomes, and the like), viral particles or other preparations comprising viral antigens, and any other antigen or fragment of an antigen, e.g., a peptide or polypeptide antigen. The antigen can also be a bacterial cell, bacterial lysate, membrane fraction from a cellular lysate, or any other source. The antigen can be expressed or produced recombinantly, or even chemically synthesized. The recombinant antigen can also be expressed on the surface of a host cell (e.g., bacteria, yeast, insect, vertebrate or mammalian cells)(e.g., expressed on the plasma membrane), can be present in a lysate, or can be purified from the lysate. Alternatively, the antigen can be encoded by nucleic acids which can be ribonucleoic acid (RNA) or deoxyribonucleic acid (DNA), that are purified or amplified from a tumor cell.

A target antigen can be present in a sample from a subject. For example, a tissue sample from a hyperproliferative or other condition in a subject can be used as a source of antigen. Such a sample can be obtained, for example, by biopsy or by surgical resection. Such an antigen can be used as a lysate or as an isolated preparation. Alternatively, a membrane preparation of cells from a subject (e.g., a cancer patient), or an established cell line also can be used as an antigen or source of antigen or nucleic acid encoding the antigen.

In some cases, the APC (e.g., APC, monocyte, activated APC) is contacted with 1×10² or more target cells (e.g., 1×10³ or more cells, 1×10⁴ or more cells, 1×10⁵ or more cells, or 1×10⁶ or more target cells) (i.e., cells that include a target antigen)(e.g., cancer cells from the individual). In some cases, APC (e.g., APC, monocyte, activated APC) is contacted with target cells in a range of from 1×10² to 1×10¹⁰ cells (1×10² to 1×10⁸ cells, 1×10³ to 1×10⁷ cells, 1×10⁴ to 1×10⁶ cells, 5×10⁴ to 5×10⁵ cells, or 1×10⁵ cells).

In some cases, where the target antigen is a cell and where the cells are lysed to produce a lysate, the APC (e.g., APC, monocyte, activated APC) can be contacted with lysate (e.g., a lysate having surface expressed antigens; an unfractionated lysate; a lysate that has been enriched for surface expressed antigens, i.e., plasma membrane expressed antigens; a membrane enriched fraction of a lysate; etc.) from 1×10² or more cells (e.g., cancer cells from an individual) (e.g., 1×10³ or more cells, 1×10⁴ or more cells, 1×10⁵ or more cells, or 1×10⁶ or more cells).

In some cases, the methods include verifying that the APC, have been loaded (i.e., verifying the presence of loaded APC). Any convenient method for determining whether an APC, is a loaded APC, can be used. For example, in some cases, the morphology alone of the APC, is indicative that the APC, is loaded. In some cases, upregulation of MHCII (e.g., HLA-DR), CD40, and/or CD86 is indicative that an APC, is loaded. For example, in some cases, upregulation of MHCII (e.g., HLA-DR) and/or CD86 is indicative that a DC is loaded. In some cases, upregulation of CD40 and/or CD86 is indicative that a DC is loaded. For example, an increase in the fraction (%) of DC that co-express CD40 and CD86 after contacting APC; relative to the fraction prior to contact, or relative to the fraction in control APC (e.g., APC not contacted in the same way and/or with the same composition); can be considered to be indicative that APC are loaded.

### Contacting a T Cell

In some cases, an activated APC (e.g., an activated and loaded APC) is used to cross-prime a naive T cell into an antigen specific effector cell. For example, in some cases, an activated APC is contacted with a naive T-cell, resulting in cross priming and the production of an antigen specific effector cell. An activated APC can be contacted with a naive T-cell in vivo (e.g., by introducing the activated APC into an individual). On the other hand, an activated APC can be contacted with a naive T-cell in vivo and/or ex vivo. In some such cases, the method (e.g., a method of treatment) includes a stop of introducing into an individual an antigen specific effector T cell that was generated by contacting a naive T cell in vitro or ex vivo with an activated APC.

In some embodiments, a naive T cell is contacted with an activated APC, e.g., an activated and loaded APC. During contact, the activated APC presents antigens to the T cell to produce a contacted T cell (e.g, which can be referred to as a cross-primed antigen specific effector cell, a cross-primed antigen specific effector T cell, etc.), and the contacted T cell generates an immune response specific to the presented antigens. The T cells can be CD4+ T cells, CD8+ T cells, or a combination of CD4+ and CD8+ T cells.

Contacting a T cell with an activated APC, e.g., an activated and loaded APC, can be in vitro or in vivo. Thus, contacting a T cell (e.g., contacting a naive T cell) encompasses both in vitro and in vivo contact. If the contact is in vivo, activated APCs, can be administered to the individual and the APCs then contact endogenous T cells of the individual to induce an immune response. Thus, a step of “contacting a naive T cell of an individual with an activated APC, when performed in vivo, can in some cases be written: “introducing into an individual an activated APC.”

If the contact is in vitro, then an autologous naive T cell (e.g., a population of autologous T cells) from the individual can be contacted with an activated APC to produce a contacted T cell (e.g., a population of contacted T cells)(cross-primed antigen specific effector cells). A T cell can be contacted with an activated APC for a period of time sufficient to activate the T cell such that the T cell will induce an immune response when administered to the individual. T cells (either prior to or after contact with an activated APC) can be expanded in vitro and/or modified (e.g., genetically modified) prior to being administered to the individual.

In some cases, a T cell is contacted in vitro with an activated APC for a period of time in a range of from 5 minutes to 24 hours (e.g., 5 minutes to 18 hours, 5 minutes to 12 hours, 5 minutes to 8 hours, 5 minutes to 6 hours, 5 minutes to 4 hours, 5 minutes to 2 hours, 5 minutes to 60 minutes, 5 minutes to 45 minutes, 5 minutes to 30 minutes, 15 minutes to 18 hours, 15 minutes to 12 hours, 15 minutes to 8 hours, 15 minutes to 6 hours, 15 minutes to 4 hours, 15 minutes to 2 hours, 15 minutes to 60 minutes, 15 minutes to 45 minutes, 15 minutes to 30 minutes, 20 minutes to 18 hours, 20 minutes to 12 hours, 20 minutes to 8 hours, 20 minutes to 6 hours, 20 minutes to 4 hours, 20 minutes to 2 hours, 20 minutes to 60 minutes, 20 minutes to 45 minutes, 30 minutes to 18 hours, 30 minutes to 12 hours, 30 minutes to 8 hours, 30 minutes to 6 hours, 30 minutes to 4 hours, 30 minutes to 2 hours, 30 minutes to 60 minutes, 30 minutes to 45 minutes, 45 minutes to 18 hours, 45 minutes to 12 hours, 45 minutes to 8 hours, 45 minutes to 6 hours, 45 minutes to 4 hours, 45 minutes to 2 hours, 45 minutes to 60 minutes, 1 hour to 18 hours, 1 hour to 12 hours, 1 hour to 8 hours, 1 hour to 6 hours, 1 hour to 4 hours, 1 hour to 2 hours, or 1 hour to 90 minutes).

In some cases, a population of T cells (e.g., naive T cells)(e.g., 1×10² or more cells (e.g., 1×10³ or more cells, 1×10⁴ or more cells, 1×10⁵ or more cells, or 1×10⁶ or more cells)) is contacted in vitro with an activated APC (e.g., a population of activated APCs, a population having activated APCs, etc.). In some cases, a population of T cells (e.g., in a range of from 1×10² to 1×10¹⁰ cells (1×10² to 1×10⁸ cells, 1×10³ to 1×10⁷ cells, 1×10⁴ to 1×10⁶ cells, 5×10⁴ to 5×10⁵ cells, or 1×10⁵ cells)) is contacted in vitro with an activated APC (e.g., a population of activated APCs, a population having activated APCs, etc.). In some cases, a T cell (e.g., a population of T cells) is contacted with a cell population (e.g., 1×10² or more cells (e.g., 1×10³ or more cells, 1×10⁴ or more cells, 1×10⁵ or more cells, or 1×10⁶ or more cells)) having activated APCs (e.g., a cell population of activated APCs). The contacted T cell (a cross-primed antigen specific effector cell) (e.g., cells of a contacted T cell population) can be administered to an individual as described below for the “administering cells”.

In some embodiments, an autologous APC, from an individual is contacted with a subject BTN3A ectodomain polypeptide to produce a stimulated APC; an autologous target antigen (e.g., a cancer cell from the individual) is contacted with a subject antibody composition to produce an immune complex; and the stimulated APC, is contacted with the immune complex, for a period of time and at a concentration effective to induce the uptake of the target antigen (e.g., the immune complex) by the stimulated APC; thereby producing a loaded APC; and the loaded APC, is contacted with a T cell (as described in greater detail above) to produce a contacted T cell, and the contacted T cell generates an immune response specific to the presented antigens.

Administering Cells and/or Compositions

In some cases, cells (e.g., activated APCs, e.g., activated DCs, activated macrophages, activated B-cells; APCs, DCs, macrophages, B-cells; and/or contacted T cells) are cultured for a period of time prior to transplantation (i.e., administration to the individual). Cells (e.g., activated APCs, e.g., activated DCs, activated macrophages, activated B-cells; APCs, e.g., DCs, macrophages, B-cells; and/or contacted T cells) can be provided to the individual (i.e., administered into the individual) alone or with a suitable substrate or matrix, e.g. to support their growth and/or organization in the tissue to which they are being transplanted (e.g., target organ, tumor tissue, blood stream, and the like). In some embodiments, the matrix is a scaffold (e.g., an organ scaffold). In some embodiments, 1×10³ or more cells will be administered, for example 5×10³ or more cells, 1×10⁴ or more cells, 5×10⁴ or more cells, 1×10⁵ or more cells, 5×10⁵ or more cells, 1×10⁶ or more cells, 5×10⁶ or more cells, 1×10⁷ or more cells, 5×10⁷ or more cells, 1×10⁸ or more cells, 5×10⁸ or more cells, 1×10⁹ or more cells, 5×10⁹ or more cells, or 1×10¹⁰ or more cells. In some embodiments, subject cells are administered into the individual on microcarriers (e.g., cells grown on biodegradable microcarriers).

Subject cells (e.g., activated APCs, e.g., activated DCs, activated macrophages, activated B-cells; APCs, macrophages, B-cells; and/or contacted T cells, e.g., antigen specific effector T cells) and/or compositions (e.g., a subject BTN3A ectodomain polypeptide) can be administered in any physiologically acceptable excipient (e.g., William's E medium), where the cells may find an appropriate site for survival and function (e.g., organ reconstitution). The cells and/or compositions may be introduced by any convenient method (e.g., injection, catheter, or the like). The cells and/or compositions can be encapsulated into liposomes or other biodegradable constructs.

The cells and/or compositions may be introduced to the subject (i.e., administered to the individual) via any of the following routes: parenteral, subcutaneous (s.c.), intravenous (i.v.), intracranial (i.c.), intraspinal, intraocular, intradermal (i.d.), intramuscular (i.m.), intralymphatic (i.l.), or into spinal fluid. The cells and/or compositions may be introduced by injection (e.g., systemic injection, direct local injection, local injection into or near a tumor and/or a site of tumor resection, etc.), catheter, or the like. Examples of methods for local delivery (e.g., delivery to a tumor and/or cancer site) include, e.g., by bolus injection, e.g. by a syringe, e.g. into a joint, tumor, or organ, or near a joint, tumor, or organ; e.g., by continuous infusion, e.g. by cannulation, e.g. with convection (see e.g. US Application No. 20070254842, incorporated here by reference); or by implanting a device upon which cells have been reversably affixed (see e.g. US Application Nos. 20080081064 and 20090196903, incorporated herein by reference).

The number of administrations of treatment to a subject may vary. Introducing cells and/or compositions into an individual may be a one-time event; but in certain situations, such treatment may elicit improvement for a limited period of time and require an on-going series of repeated treatments. In other situations, multiple administrations of cells and/or compositions may be required before an effect is observed. As will be readily understood by one of ordinary skill in the art, the exact protocols depend upon the disease or condition, the stage of the disease and parameters of the individual being treated.

A “therapeutically effective dose” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of cells and/or compositions is an amount that is sufficient, when administered to (e.g., transplanted into) the individual, to palliate, ameliorate, stabilize, reverse, prevent, slow or delay the progression of the disease state (e.g., tumor size, tumor growth, tumor presence, cancer presence, etc.) by, for example, inducing an immune response against antigenic cells (e.g., cancer cells).

In some embodiments, a therapeutically effective dose of cells (e.g., activated APC; contacted T cells; etc.) is 1×10³ or more cells (e.g., 5×10³ or more, 1×10⁴ cells, 5×10⁴ or more, 1×10⁵ or more, 5×10⁵ or more, 1×10⁶ or more, 2×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more).

In some embodiments, a therapeutically effective dose of cells is in a range of from 1×10³ cells to 1×10¹⁰ cells (e.g, from 5×10³ cells to 1×10¹⁰ cells, from 1×10⁴ cells to 1×10¹⁰ cells, from 5×10⁴ cells to 1×10¹⁰ cells, from 1×10⁵ cells to 1×10¹⁰ cells, from 5×10⁵ cells to 1×10¹⁰ cells, from 1×10⁶ cells to 1×10¹⁰ cells, from 5×10⁶ cells to 1×10¹⁰ cells, from 1×10⁷ cells to 1×10¹⁰ cells, from 5×10⁷ cells to 1×10¹⁰ cells, from 1×10⁸ cells to 1×10¹⁰ cells, from 5×10⁸ cells to 1×10¹⁰, from 5×10³ cells to 5×10⁹ cells, from 1×10⁴ cells to 5×10⁹ cells, from 5×10⁴ cells to 5×10⁹ cells, from 1×10⁵ cells to 5×10⁹ cells, from 5×10⁵ cells to 5×10⁹ cells, from 1×10⁶ cells to 5×10⁹ cells, from 5×10⁶ cells to 5×10⁹ cells, from 1×10⁷ cells to 5×10⁹ cells, from 5×10⁷ cells to 5×10⁹ cells, from 1×10⁸ cells to 5×10⁹ cells, from 5×10⁸ cells to 5×10⁹, from 5×10³ cells to 1×10⁹ cells, from 1×10⁴ cells to 1×10⁹ cells, from 5×10⁴ cells to 1×10⁹ cells, from 1×10⁵ cells to 1×10⁹ cells, from 5×10⁵ cells to 1×10⁹ cells, from 1×10⁶ cells to 1×10⁹ cells, from 5×10⁶ cells to 1×10⁹ cells, from 1×10⁷ cells to 1×10⁹ cells, from 5×10⁷ cells to 1×10⁹ cells, from 1×10⁸ cells to 1×10⁹ cells, from 5×10⁸ cells to 1×10⁹, from 5×10³ cells to 5×10⁸ cells, from 1×10⁴ cells to 5×10⁸ cells, from 5×10⁴ cells to 5×10⁸ cells, from 1×10⁵ cells to 5×10⁸ cells, from 5×10⁵ cells to 5×10⁸ cells, from 1×10⁶ cells to 5×10⁸ cells, from 5×10⁶ cells to 5×10⁸ cells, from 1×10⁷ cells to 5×10⁸ cells, from 5×10⁷ cells to 5×10⁸ cells, or from 1×10⁸ cells to 5×10⁸ cells).

In some embodiments, the concentration of cells (e.g., activated APCs; contacted T cells; and the like) to be administered is in a range of from 1×10⁵ cells/ml to 1×10⁹ cells/ml (e.g., from 1×10⁵ cells/ml to 1×10⁸ cells/ml, from 5×10⁵ cells/ml to 1×10⁸ cells/ml, from 5×10⁵ cells/ml to 5×10⁷ cells/ml, from 1×10⁶ cells/ml to 1×10⁸ cells/ml, from 1×10⁶ cells/ml to 5×10⁷ cells/ml, from 1×10⁶ cells/ml to 1×10⁷ cells/ml, from 1×10⁶ cells/ml to 6×10⁶ cells/ml, or from 2×10⁶ cells/ml to 8×10⁶ cells/ml).

In some embodiments, the concentration of cells (e.g., activated APCs; contacted T cells; and the like) to be administered is 1×10⁵ cells/ml or more (e.g., 1×10⁵ cells/ml or more, 2×10⁵ cells/ml or more, 3×10⁵ cells/ml or more, 4×10⁵ cells/ml or more, 5×10⁵ cells/ml or more, 6×10⁵ cells/ml or more, 7×10⁵ cells/ml or more, 8×10⁵ cells/ml or more, 9×10⁵ cells/ml or more, 1×10⁶ cells/ml or more, 2×10⁶ cells/ml or more, 3×10⁶ cells/ml or more, 4×10⁶ cells/ml or more, 5×10⁶ cells/ml or more, 6×10⁶ cells/ml or more, 7×10⁶ cells/ml or more, or 8×10⁶ cells/ml or more).

The cells and/or compositions of this disclosure can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

Cells of the subject methods may be genetically modified to enhance survival, control proliferation, and the like. Cells may be genetically altered by transfection or transduction with a suitable vector, homologous recombination, or other appropriate technique, so that they express a gene of interest. In some embodiments, a selectable marker is introduced, to provide for greater purity of the desired cell.

In some cases, a subject BTN3A ectodomain polypeptide can be used as an adjuvant (e.g., to enhance the efficacy of a vaccine, e.g., for cancer or for an infectious disease). Methods are provided for enhancing immune responses to an antigen. The antigen can be from any source (e.g., a human, a non-human animal, a plant, a bacterial cell, an archaeal cell, a fungus, a virus, a parasite, a cancer cell, etc.). The individual into which a BTN3A ectodomain polypeptide is introduced as an adjuvant can be any multicellular organism (e.g., a human, a non-human animal, a mammal, a primate, a rodent, etc.). In some cases, the antigen is a vaccine (e.g., a cancer vaccine, a vaccine directed at a particular disease, pathogen, and/or virus). For example, in some cases, the vaccine is directed at Tuberculosis, Malaria, Human Immunodeficiency Virus (HIV), RotaVirus, Herpes Simplex Virus (HSV), or Cytomegalovirus (CMV).

The subject therapeutic agents (e.g., a subject BTN3A ectodomain polypeptide) can activate immune cells (e.g., monocytes and APCs such as dendritic cells, macrophages, and B cells; and therefore T cells), and therefore enhance immune cell functions such as inhibiting cancer cell growth and/or viral infection, and restore immune surveillance and immune memory function to treat human disease. Examples of symptoms, illnesses, and/or diseases that can be treated with a subject BTN3A ectodomain polypeptide include, but are not limited to cancer (any form of cancer, including but not limited to: carcinomas, soft tissue tumors, sarcomas, teratomas, melanomas, leukemias, lymphomas, brain cancers, solid tumors, mesothelioma (MSTO), etc.); infection (e.g., chronic infection); and/or an immunological disease or disorder (e.g., an inflammatory disease, e.g., multiple sclerosis, arthritis, and the like). An individual with primary or combined immunodeficiency (e.g., an individual with Bruton's Agammaglobulinemia, Common Variable Immunodeficiency, X linked SCJD, etc.) can also be treated with a subject BTN3A ectodomain polypeptide. For example, in some cases, a subject BTN3A ectodomain polypeptide can be used as an immune stimulant (e.g., used for immunopotentiation). Any disease, disorder or ailment that involves immunosuppression can be treated using a subject BTN3A ectodomain polypeptide.

The terms “co-administration”, “co-administer”, and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

For example, “concomitant administration” of a therapeutic drug (e.g., cancer therapeutic drug, e.g., a tumor-directed antibody; a therapeutic drug to treat an infection; etc.) with a subject BTN3A polypeptide (e.g., as a pharmaceutical composition) of the present disclosure means administration with a subject BTN3A polypeptide at such time that both the therapeutic drug and the subject BTN3A polypeptide will have a therapeutic effect. Such concomitant administration may involve concurrent (i.e. at the same time), prior, or subsequent administration of the therapeutic drug with respect to the administration of the BTN3A polypeptide. A person of ordinary skill in the art would have no difficulty determining the appropriate timing, sequence and dosages of administration for particular drugs and compositions of the present disclosure.

Because a subject BTN3A ectodomain polypeptide activates APCs and stimulates the immune system, in some cases, the agent which is co-administered with the BTN3A ectodomain polypeptide is administered at a dose that is lower when the agent is administered in the absence of the BTN3A ectodomain polypeptide. For example, if the agent is known to have side effects at higher doses, co-administration with a BTN3A ectodomain polypeptide can allow for dosage to be adjusted to reduce the level of side effects. In some cases, the agent which is co-administered with the BTN3A ectodomain polypeptide is administered at a dose that is considered sub-therapeutic when the agent is administered in the absence of the BTN3A ectodomain polypeptide. In some cases, the agent which is co-administered with the BTN3A ectodomain polypeptide is an agent that is not effective when administered without the BTN3A ectodomain polypeptide. Thus, co-administration with a BTN3A ectodomain polypeptide can render agents effective that in the past have been considered to be ineffective.

In some embodiments, a subject BTN3A ectodomain polypeptide is administered in combination (co-administration) with another agent, e.g., an immune stimulant, an APC stimulatory agent, an agent to treat chronic infection, a cytotoxic agent, a vaccine, a BiTE (bispecific T cell engaging) antibody, a chimeric antigen receptor (CAR)/TCR-engineered T cell, and the like. One example class of cytotoxic agents are chemotherapeutic agents. Exemplary chemotherapeutic agents include, but are not limited to, an anti-CD47 antibody, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine and vinorelbine tartrate.

Suitable agents for co-administration with a subject BTN3A ectodomain polypeptide include agents that block the binding of CD47 on a first cell to SIRPα on a second cell (e.g., a binding protein (or fragment thereof) that binds to CD47, e.g., an anti-CD47 antibody, a SIRPα polypeptide derived from the ectodomain of SIRPα, etc.; a binding protein (or fragment thereof) that binds to SIRPα, e.g., an anti-SIRPα antibody, a CD47 polypeptide derived from the ectodomain of CD47, etc.).

Suitable agents for co-administration with a subject BTN3A ectodomain polypeptide include agents that block the binding of PD-1 on a first cell to PDL-1 on a second cell (e.g., a binding protein (or fragment thereof) that binds to PD-1, e.g., an anti-PD-1 antibody, a PD-L1 polypeptide derived from the ectodomain of PD-L1, etc.; a binding protein (or fragment thereof) that binds to PD-L1, e.g., an anti-PD-L1 antibody, a PD-1 polypeptide derived from the ectodomain of PD-1, etc.)

A subject BTN3A ectodomain polypeptide can be co-administered with an agent (e.g., an antibody) that specifically binds to a target molecule other than BTN3A (e.g., CD19, CD20, CD22, CD24, CD25, CD30, CD33, CD38, CD44, CD52, CD56, CD70, CD96, CD97, CD99, CD123, CD279 (PD-1), PD-L1, EGFR, HER2, CD117, C-Met, PTHR2, HAVCR2 (TIM3), etc.) Examples of antibodies with CDRs that provide specific binding to a cancer cell marker (and therefore can be used in a combination therapy (co-administered with a subject BTN3A ectodomain polypeptide) include, but are not limited to: CETUXIMAB (binds EGFR), PANITUMUMAB (binds EGFR), RITUXIMAB (binds CD20), TRASTUZUMAB (binds HER2), PERTUZUMAB (binds HER2), ALEMTUZUMAB (binds CD52), and BRENTUXIMAB (binds CD30).

In some cases, a subject BTN3A ectodomain polypeptide is co-administered with a T cell with an engineered T cell receptor (TCR) (such a cell is also referred to herein as a “TCR-engineered T cell”). Non-limiting suitable examples of a TCR-engineered T cell are: (i) a T cell that includes a chimeric antigen receptor (CAR); and (ii) a T cell that includes a heterologous TCR that binds to an antigen such as a cancer antigen. (TCR-engineered T cells are described in more detail in the section on introducing nucleic acids).

A subject BTN3A ectodomain polypeptide can be co-administered with any convenient immunomodulatory agent (e.g., an anti-CTLA4 antibody, an anti-PD-1 antibody, a CD40 agonist, a 4-1BB modulator (e.g., a 41BB-agonist), and the like).

In some embodiments, a subject BTN3A polypeptide is administered in combination (co-administration) with an opsonizing agent (e.g., an opsonizing antibody, an ADCC-inducing antibody). An “opsonizing agent” or an “agent that opsonizes a target cell” (e.g., an “opsonizing antibody”) is any agent that can bind to a target cell (e.g., a cancer cell, a cell harboring an intracellular pathogen, etc.) and opsonize the target cell. For example, any antibody (or fragment thereof, or antibody mimic, etc.) that can bind to a target cell, where the antibody has an FC region, is considered to be an opsonizing agent. In some cases, the opsonizing agent is an antibody that binds to a target cell (e.g., an anti-tumor antibody, an anti-cancer antibody, an anti-infection antibody, and the like). In some cases, an opsonizing agent is an antibody that induces Antibody-Dependent Cell-mediated Cytotoxicity (ADCC). Such an antibody can be referred to as an ADCC-inducing antibody (e.g., an opsonizing antibody).

A number of opsonizing agents (e.g., ADCC-inducing antibodies, opsonizing antibodies) are currently in clinical use for the treatment of cancer, and others are in varying stages of clinical development. For example, there are a number of antigens and corresponding monoclonal antibodies for the treatment of B cell malignancies. One target antigen is CD20. Rituximab is a chimeric unconjugated monoclonal antibody directed at the CD20 antigen. CD20 has an important functional role in B cell activation, proliferation, and differentiation. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Two new monoclonal antibodies targeting CD20, tositumomab and ibritumomab, have been submitted to the Food and Drug Administration (FDA). These antibodies are conjugated with radioisotopes. Alemtuzumab (Campath) is used in the treatment of chronic lymphocytic leukemia; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer.

Monoclonal antibodies useful in the compositions and methods of the disclosure that have been used in solid tumors include, without limitation, edrecolomab and trastuzumab (herceptin). Edrecolomab targets the 17-1A antigen seen in colon and rectal cancer, and has been approved for use in Europe for these indications. Trastuzumab targets the HER-2/neu antigen. This antigen is seen on 25% to 35% of breast cancers. Cetuximab (Erbitux) is also of interest for use in the methods of the disclosure. The antibody binds to the EGF receptor (EGFR), and has been used in the treatment of solid tumors including colon cancer and squamous cell carcinoma of the head and neck (SCCHN).

Combination therapies could include administration cell-specific antibodies, for example antibodies selective for tumor cell markers (i.e., anti-tumor antibodies), radiation, surgery, and/or hormone deprivation (Kwon et al., Proc. Natl. Acad. Sci U.S.A., 96: 15074-9, 1999). Angiogenesis inhibitors can also be combined with the methods of this disclosure.

In some cases, an opsonizing agent (e.g., an ADCC-inducing antibody, an anti-tumor antibody) is an antibody specific for an antigen selected from: CD20, CD52, CD38, HER-2, 17-1A, and EGFR. In some cases, treatment is accomplished by administering a combination (co-administration) of a subject BTN3A polypeptide with an opsonizing agent where the opsonizing agent is an anti-tumor antibody (e.g., an antibody that is specific for an antigen selected from: CD20, CD52, CD38, HER-2, 17-1A, and EGFR). In some cases, treatment is accomplished by administering a combination (co-administration) of a subject BTN3A polypeptide with one or more opsonizing agents where each of said one or more opsonizing agents is an anti-tumor antibody (e.g., an antibody that is specific for an antigen selected from: CD20, CD52, CD38, HER-2, 17-1A, and EGFR).

A subject BTN3A ectodomain polypeptide can be co-administered with an APC stimulatory agent, which can include, but are not limited to dendritic cell stimulatory agents, macrophage stimulatory agents, and B-cell stimulatory agents. In some cases, an APC stimulatory agent is a dendritic cell stimulatory agent. In some cases, an APC stimulatory agent is a macrophage stimulatory agent. In some cases, an APC stimulatory agent is a B-cell stimulatory agent. In some cases, an APC stimulatory agent is not a macrophage stimulatory agent. Examples of APC stimulatory agents (e.g., dendritic cell stimulatory agents, macrophage stimulatory agents, B-cell stimulatory agents) include, but are not limited to, a composition that contains (i) a Toll-like receptor (TLR) agonist; (ii) a CD40 agonist and a proinflammatory cytokine; (iii) a checkpoint molecule neutralizing compound; (iv) an indoleamine 2,3-dioxygenase (IDO) inhibitor; (v) an NFkB activator; (vi) a compound that opens calcium channels; (vii) a T cell-related co-stimulatory molecule; or (viii) a combination thereof. In some cases, the TLR agonist is CpG ODN, immunostimulatory DNA, immunostimulatory RNA, immunostimulatory oligonucleotides, Imiquimod, Resiquimod, Loxribine, Flagellin, FSL-I or LPS.

As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions.

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratoma is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Leukemias are cancers that start in blood-forming tissue, such as the bone marrow, and causes large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is effected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Sternberg cell. There are currently 6 recognized types of HL. Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

As used herein, the term “infection” refers to any state in at least one cell of an organism (i.e., a subject) is infected by an infectious agent (e.g., a subject has an intracellular pathogen infection, e.g., a chronic intracellular pathogen infection). As used herein, the term “infectious agent” refers to a foreign biological entity (i.e. a pathogen). For example, infectious agents include, but are not limited to bacteria, viruses, protozoans, and fungi. Intracellular pathogens are of particular interest. Infectious diseases are disorders caused by infectious agents. Some infectious agents cause no recognizable symptoms or disease under certain conditions, but have the potential to cause symptoms or disease under changed conditions. The subject methods can be used in the treatment of chronic pathogen infections, for example including but not limited to viral infections, e.g. retrovirus, lentivirus, hepadna virus, herpes viruses, pox viruses, human papilloma viruses, etc.; intracellular bacterial infections, e.g. Mycobacterium, Chlamydophila, Ehrlichia, Rickettsia, Brucella, Legionella, Francisella, Listeria, Coxiella, Neisseria, Salmonella, Yersinia sp, Helicobacter pylori etc.; and intracellular protozoan pathogens, e.g. Plasmodium sp, Trypanosoma sp., Giardia sp., Toxoplasma sp., Leishmania sp., etc.

Infectious diseases that can be treated using a subject BTN3A ectodomain polypeptide include but are not limited to: HIV, Influenza, Herpes, Giardia, Malaria, Leishmania, the pathogenic infection by the virus Hepatitis (A, B, & C), herpes virus (e.g., VZV, HSV-I, HAV-6, HSV-II, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus, pathogenic infection by the bacteria chlamydia, rickettsial bacteria, mycobacteria, staphylococci, streptococci, pneumonococci, meningococci and conococci, klebsiella, proteus, serratia, pseudomonas, E. coli, legionella, diphtheria, salmonella, bacilli, cholera, tetanus, botulism, anthrax, plague, leptospirosis, and Lyme's disease bacteria, pathogenic infection by the fungi Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum, and pathogenic infection by the parasites Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and/or Nippostrongylus brasiliensis.

A subject BTN3A ectodomain polypeptide can facilitate and/or stimulate cytokine and/or chemokine production by immune cells. For example, the presence of an immune complex (i.e., an antigen-antibody complex) interacting with an immune cell activates the immune cell and induces cytokine production by the immune cell. A subject BTN3A ectodomain polypeptide can be used for altering immunoresponsiveness of an immune cell and thereby may be useful for treating or preventing an immunological disease or disorder (e.g., a disorder associated with immunosuppression). In other words, a subject BTN3A ectodomain polypeptide can be used for immunopotentiation (stimulation of the immune system) as an agent that simulates the immune system.

The methods above include administering to an individual in need of treatment a therapeutically effective amount or an effective dose of a subject BTN3A ectodomain polypeptide, including without limitation combinations of a BTN3A ectodomain polypeptide with a drug (e.g., a chemotherapeutic drug, an ADCC-inducing antibody, an opsonizing agent, a tumor-specific antibody, an anti-inflammatory drug, a drug to treat infection, an immunostimulant, i.e., an immunopotentiator, an agent that simulates the immune system, etc.).

Effective doses of the therapeutic entity of the present disclosure (e.g., BTN3A ectodomain polypeptide), e.g. for the treatment of cancer, vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every day, or once every two days, or once every week, or once every two weeks, or once a month, or once every two months, or once every 3 to 6 months. Therapeutic entities of the present disclosure are usually administered on multiple occasions. Intervals between single dosages can be weekly, monthly or yearly. Intervals can also be irregular as indicated by measuring blood levels of the therapeutic entity in the patient. Alternatively, therapeutic entities of the present disclosure can be administered as a sustained release formulation, in which case less frequent administration is required. Dosage and frequency vary depending on the half-life of the polypeptide in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still other embodiments, methods of the present disclosure include treating, reducing or preventing any of the above discussed conditions, ailments, and/or diseases (e.g., tumor growth, tumor metastasis or tumor invasion of cancers including lymphomas, leukemias, carcinomas, melanomas, glioblastomas, sarcomas, myelomas, etc.). For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Subject BTN3A ectodomain polypeptides can be used in vitro in binding assays in which they can be utilized in liquid phase or bound to a solid phase carrier. In addition, the polypeptides can be detectably labeled in various ways. Examples of types of assays which can utilize BTN3A ectodomain polypeptides are flow cytometry, e.g. FACS, MACS, histochemistry, competitive and non-competitive immunoassays in either a direct or indirect format; and the like. Detection of a BTN3A target molecule (e.g., receptor) using a BTN3A ectodomain polypeptide can be done with assays which are run in either the forward, reverse, or simultaneous modes, including histochemical assays on physiological samples.

Subject BTN3A ectodomain polypeptides can be bound to many different carriers and used to detect the presence of cells expressing a BTN3A target molecule (e.g., receptor). Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding proteins, or will be able to ascertain such, using routine experimentation.

There are many different labels and methods of labeling known to those of ordinary skill in the art. Examples of the types of labels which can be used in the present disclosure include but are not limited to enzymes, radioisotopes, fluorescent compounds, colloidal metals, nanoparticles, chemiluminescent compounds, and bio-luminescent compounds. Those of ordinary skill in the art will know of other suitable labels for binding to the polypeptides of the disclosure, or will be able to ascertain such, using routine experimentation. Furthermore, the binding of these labels to the polypeptides of the disclosure can be done using standard techniques common to those of ordinary skill in the art.

The imaging conjugates of BTN3A ectodomain polypeptides can be administered to the subject in a series of more than one administration. The imaging conjugate compositions may be administered at an appropriate time before the visualization technique. For example, administration within an hour before direct visual inspection may be appropriate, or administration within twelve hours before a PET or MRI scan may be appropriate. Care should be taken, however, to not allow too much time to pass between administration and visualization, as the imaging compound may eventually be cleared from the patient's system.

Compositions of the disclosure (e.g., for the treatment of cancer, chronic infection, immunosuppression, inflammation, etc.) can be administered locally (e.g., into a tumor, at the site of a tumor resection, at the site of infection, etc.) or systemically (e.g., orally, intravenously, etc.). Compositions of the disclosure (e.g., for the treatment of cancer, chronic infection, immunosuppression, inflammation, etc.) can be administered by parenteral, topical, intravenous, intratumoral, oral, subcutaneous, intraarterial, intracranial, intraperitoneal, intranasal or intramuscular means. For example, such means can include, but are not limited to: parenteral injection, intramuscular injection, intraperitoneal injection, intravenous injection, subcutaneous injection, intratumoral injection, inhalation, rectal delivery, vaginal delivery, nasal delivery, oral delivery, opthamalical delivery, topical delivery, transdermal delivery, and intradermal delivery. A typical route of administration is intravenous or intratumoral, although other routes can be equally effective.

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the BTN3A ectodomain polypeptides described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

Identifying and/or Generating a High Affinity BTN3A Ectodomain Polypeptide

Also provided are methods of identifying and/or generating a high affinity BTN3A ectodomain polypeptide (a BTN3A ectodomain polypeptide that includes a high affinity BTN3A ectodomain). Such methods can include mutating a BTN3A ectodomain amino acid sequence such that the newly produced BTN3A ectodomain has increased affinity for the target molecule of the BTN3A ectodomain polypeptide (i.e., in increased affinity for the molecule to which the BTN3A ectodomain specifically binds). Such methods can include a step of mutagenizing a starting sequence (e.g., nucleic acid sequence encoding a BTN3A ectodomain) to generate one or more nucleic acids comprising nucleotides sequences that encode candidate high affinity BTN3A ectodomains. As such, in some cases, a method of generating (and/or identifying) a high affinity BTN3A ectodomain polypeptide includes a step of mutagenizing a nucleic acid that includes a nucleotide sequence encoding a BTN3A ectodomain to produce a nucleic acid that includes a nucleotide sequence encoding a candidate high affinity BTN3A ectodomain. In some cases, a method of generating (and/or identifying) a high affinity BTN3A ectodomain polypeptide includes a step of providing a nucleic acid that includes a nucleotide sequence encoding a candidate high affinity BTN3A ectodomain.

To test whether a given candidate high affinity BTN3A ectodomain is in fact a high affinity BTN3A ectodomain, any convenient assay can be used to assess whether the candidate has increased affinity for the target molecule (the molecule bound by the wild type BTN3A protein) relative to the affinity of a corresponding wild type sequence for the target molecule. For example, a direct binding assay can be used to assess the binding affinity of the candidate compared to the starting protein (the protein encoded by the nucleic acid that served as the starting point for mutation in order to arrive at a nucleic acid encoding the candidate high affinity sequence). For example, an assay can be used to directly detect binding to the target molecule (e.g., the molecule to which the BTN3A ectodomain specifically binds), and/or to a cell that expresses the target molecule (e.g., an APC cell). In some cases, the target molecule bound by the BTN3A ectodomain is selected from: LTβR, FLT1, HLA-E, CD163, and ROR2. In some cases, the target molecule bound by the BTN3A ectodomain is LTβR. In some cases, the target molecule bound by the BTN3A ectodomain is FLT1. In some cases, the target molecule bound by the BTN3A ectodomain is HLA-E. In some cases, the target molecule bound by the BTN3A ectodomain is CD163. In some cases, the target molecule bound by the BTN3A ectodomain is ROR2.

Thus, in some cases a high affinity BTN3A ectodomain binds with higher affinity (compared to the affinity with which a corresponding wild type BTN3A ectodomain binds) to one or more molecules selected from: LTβR, FLT1, HLA-E, CD163, and ROR2. In some cases a high affinity BTN3A ectodomain binds with higher affinity to LTβR than the affinity with which a corresponding wild type BTN3A ectodomain binds to LTβR. In some cases a high affinity BTN3A ectodomain binds with higher affinity to FLT1 than the affinity with which a corresponding wild type BTN3A ectodomain binds to FLT1. In some cases a high affinity BTN3A ectodomain binds with higher affinity to HLA-E than the affinity with which a corresponding wild type BTN3A ectodomain binds to HLA-E. In some cases a high affinity BTN3A ectodomain binds with higher affinity to CD163 than the affinity with which a corresponding wild type BTN3A ectodomain binds to CD163. In some cases a high affinity BTN3A ectodomain binds with higher affinity to ROR2 than the affinity with which a corresponding wild type BTN3A ectodomain binds to ROR2.

Thus, in some embodiments, a binding assay can be used to assess the binding affinity of the candidate high affinity BTN3A ectodomain polypeptide for a target molecule compared to the affinity with which a corresponding wild type BTN3A ectodomain binds to the target molecule, where the target molecule is selected from: LTβR, FLT1, HLA-E, CD163, and ROR2. In some cases, a binding assay can be used to assess the binding affinity of the candidate high affinity BTN3A ectodomain polypeptide for LTβR compared to the affinity with which a corresponding wild type BTN3A ectodomain binds to LTβR. In some cases, a binding assay can be used to assess the binding affinity of the candidate high affinity BTN3A ectodomain polypeptide for FLT1 compared to the affinity with which a corresponding wild type BTN3A ectodomain binds to FLT1. In some cases, a binding assay can be used to assess the binding affinity of the candidate high affinity BTN3A ectodomain polypeptide for HLA-E compared to the affinity with which a corresponding wild type BTN3A ectodomain binds to HLA-E. In some cases, a binding assay can be used to assess the binding affinity of the candidate high affinity BTN3A ectodomain polypeptide for CD163 compared to the affinity with which a corresponding wild type BTN3A ectodomain binds to CD163. In some cases, a binding assay can be used to assess the binding affinity of the candidate high affinity BTN3A ectodomain polypeptide for ROR2 compared to the affinity with which a corresponding wild type BTN3A ectodomain binds to ROR2.

To test whether a given candidate high affinity BTN3A ectodomain is in fact a high affinity BTN3A ectodomain, any convenient assay can be used to assess whether the candidate has increased affinity for a target molecule relative to a corresponding wild type sequence. For example, a direct binding assay can be used to assess the binding affinity of the candidate (to the target molecule) compared to the starting protein.

Binding can be determined by any convenient method, for example, measuring the ability of an unlabeled BTN3A ectodomain polypeptide to compete with a labeled BTN3A ectodomain (e.g., a labeled native BTN3A ectodomain polypeptide, as defined above) for binding to a binding partner (e.g., a particular protein, an APC cell, etc.). Accordingly, relative biding can be assessed by comparing the results using a candidate unlabeled high-affinity BTN3A ectodomain polypeptide to results using an unlabeled native BTN3A ectodomain polypeptide (as defined above, a BTN3A ectodomain polypeptide that does not have an amino acid change relative to the corresponding sequence of a corresponding wild type BTN3A protein).

In some cases, one or more functional assays can be used (in addition to or instead of direct binding assays) that do not directly measure binding (e.g., binding affinity), but can be considered a biological functional response to binding (e.g., a biological readout that is a result of binding). For example, such an assay can include one or more assays that measure the activity of APCs. For example, such assays can include, but are not limited to: (i) assays that measure secretion of one or more helper cytokines (e.g., two or more, three or more, four or more, five or more helper cytokines) from APCs, (ii) assays that measure the production of one or more costimulatory molecules (e.g., two or more, three or more, four or more, five or more costimulatory molecules) by APCs, and (iii) assays that measure one or more downstream effector functions (e.g., two or more, three or more, four or more, five or more downstream effector functions) of APCs to elicit an immune response. For each of the three assay types listed above, an increase is associated with an increase in APC activity. For example, an increase in the level of APC activation is associated with an increased secretion of one or more helper cytokines (e.g., two or more, three or more, four or more, five or more helper cytokines) from the APC, an increase in the production of one or more costimulatory molecules (e.g., two or more, three or more, four or more, five or more costimulatory molecules), and/or an increase in one or more downstream effector functions (e.g., two or more, three or more, four or more, five or more downstream effector functions) of APCs to elicit an immune response.

In some cases, a high affinity BTN3A ectodomain will be able to produce an increased activity in one or more (e.g., two or more, or all three) of the three listed assay types, compared to the activity produced by the control molecule (e.g., the control non-mutated BTN3A ectodomain) when the control and the candidate BTN3A ectodomain (a candidate high affinity BTN3A ectodomain) are provided at the same or similar concentration. In some cases, a high affinity BTN3A ectodomain will be able to produce comparable activity (e.g., the same or similar level of activity) in one or more (e.g., two or more, or all three) of the three listed assay types, compared to the activity produced by the control molecule (e.g., the control non-mutated BTN3A ectodomain) when the candidate BTN3A ectodomain (a candidate high affinity BTN3A ectodomain) is provided at a reduced concentration compared to the control.

Thus, for example, in some cases, a polypeptide having a high affinity BTN3A ectodomain (e.g., a subject high affinity BTN3A ectodomain polypeptide) can be identified by using one or more assays selected from: (i) an assay that measures secretion of one or more helper cytokines (e.g., two or more, three or more, four or more, five or more helper cytokines) from APCs, (ii) an assay that measures the production of one or more costimulatory molecules (e.g., two or more, three or more, four or more, five or more costimulatory molecules) by APCs, and (iii) an assay that measures one or more downstream effector functions (e.g., two or more, three or more, four or more, five or more downstream effector functions) of APCs to elicit an immune response. For example, in some cases a candidate high affinity BTN3A ectodomain polypeptide can be determined to be a high affinity BTN3A ectodomain polypeptide if the candidate high affinity BTN3A ectodomain polypeptide elicits one or more of: (i) an increase in secretion of one or more helper cytokines (e.g., two or more, three or more, four or more, five or more helper cytokines) from APCs, (ii) an increase in the production of one or more costimulatory molecules (e.g., two or more, three or more, four or more, five or more costimulatory molecules) by APCs, and (iii) an increase in one or more downstream effector functions (e.g., two or more, three or more, four or more, five or more downstream effector functions) of APCs to elicit an immune response.

In some cases, a polypeptide having a high affinity BTN3A ectodomain (e.g., a subject high affinity BTN3A ectodomain polypeptide) can be identified by using two or more selected from: (i) an assay that measures secretion of one or more helper cytokines (e.g., two or more, three or more, four or more, five or more helper cytokines) from APCs, (ii) an assay that measures the production of one or more costimulatory molecules (e.g., two or more, three or more, four or more, five or more costimulatory molecules) by APCs, and (iii) an assay that measures one or more downstream effector functions (e.g., two or more, three or more, four or more, five or more downstream effector functions) of APCs to elicit an immune response. For example, in some cases a candidate high affinity BTN3A ectodomain polypeptide can be determined to be a high affinity BTN3A ectodomain polypeptide if the candidate high affinity BTN3A ectodomain polypeptide elicits two or more of: (i) an increase in secretion of one or more helper cytokines (e.g., two or more, three or more, four or more, five or more helper cytokines) from APCs, (ii) an increase in the production of one or more costimulatory molecules (e.g., two or more, three or more, four or more, five or more costimulatory molecules) by APCs, and (iii) an increase in one or more downstream effector functions (e.g., two or more, three or more, four or more, five or more downstream effector functions) of APCs to elicit an immune response.

In some cases, a polypeptide having a high affinity BTN3A ectodomain (e.g., a subject high affinity BTN3A ectodomain polypeptide) can be identified by using the following three assays: (i) an assay that measures secretion of one or more helper cytokines (e.g., two or more, three or more, four or more, five or more helper cytokines) from APCs, (ii) an assay that measures the production of one or more costimulatory molecules (e.g., two or more, three or more, four or more, five or more costimulatory molecules) by APCs, and (iii) an assay that measures one or more downstream effector functions (e.g., two or more, three or more, four or more, five or more downstream effector functions) of APCs to elicit an immune response. For example, in some cases a candidate high affinity BTN3A ectodomain polypeptide can be determined to be a high affinity BTN3A ectodomain polypeptide if the candidate high affinity BTN3A ectodomain polypeptide elicits: (i) an increase in secretion of one or more helper cytokines (e.g., two or more, three or more, four or more, five or more helper cytokines) from APCs, (ii) an increase in the production of one or more costimulatory molecules (e.g., two or more, three or more, four or more, five or more costimulatory molecules) by APCs, and (iii) an increase in one or more downstream effector functions (e.g., two or more, three or more, four or more, five or more downstream effector functions) of APCs to elicit an immune response.

In some cases, a high affinity BTN3A ectodomain polypeptide has an affinity for the binding partner (e.g., target molecule, target cell, APC, etc.) that is increased by 10% or more (e.g., increased by 20% or more, 30% or more, 40% or more, 50% or more, 60% or more, 70% or more, 80% or more, 90% or more, 100% or more, 150% or more, 200% or more, 250% or more, 300% or more, 400% or more, or 500% or more) relative to the affinity of a control polypeptide (having a corresponding wild type amino acid sequence) for the binding partner.

In some cases, a high affinity BTN3A ectodomain polypeptide induces an increase in the secretion of one or more helper cytokines (e.g., two or more, three or more, four or more, five or more helper cytokines) from APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the secretion of helper cytokines from APCs induced by a control polypeptide (having a corresponding wild type amino acid sequence).

In some cases, a high affinity BTN3A ectodomain polypeptide induces an increase in the production of one or more costimulatory molecules (e.g., two or more, three or more, four or more, five or more costimulatory molecules) by APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the production of costimulatory molecules induced by a control polypeptide (having a corresponding wild type amino acid sequence).

In some cases, a high affinity BTN3A ectodomain polypeptide induces an increase in one or more of the downstream effector functions of APCs (e.g., to elicit an immune response) by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the downstream effector functions of APCs (to elicit an immune response) induced by a control polypeptide (having a corresponding wild type amino acid sequence).

In some cases, a high affinity BTN3A ectodomain polypeptide (a) induces an increase in the secretion of one or more helper cytokines (e.g., two or more, three or more, four or more, five or more helper cytokines) from APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the secretion of helper cytokines from APCs induced by a control polypeptide (having a corresponding wild type amino acid sequence); and (b) induces an increase in the production of one or more costimulatory molecules (e.g., two or more, three or more, four or more, five or more costimulatory molecules) by APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the production of costimulatory molecules induced by a control polypeptide (having a corresponding wild type amino acid sequence).

In some cases, a high affinity BTN3A ectodomain polypeptide (a) induces an increase in the secretion of one or more helper cytokines (e.g., two or more, three or more, four or more, five or more helper cytokines) from APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the secretion of helper cytokines from APCs induced by a control polypeptide (having a corresponding wild type amino acid sequence); and (b) induces an increase in the one or more downstream effector functions (e.g., two or more, three or more, four or more, five or more downstream effector functions) of APCs (to elicit an immune response) by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the downstream effector functions of APCs (to elicit an immune response) induced by a control polypeptide (having a corresponding wild type amino acid sequence).

In some cases, a high affinity BTN3A ectodomain polypeptide (a) induces an increase in the production of one or more costimulatory molecules (e.g., two or more, three or more, four or more, five or more costimulatory molecules) by APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the production of costimulatory molecules induced by a control polypeptide (having a corresponding wild type amino acid sequence); and (b) induces an increase in the one or more downstream effector functions (e.g., two or more, three or more, four or more, five or more downstream effector functions) of APCs (to elicit an immune response) by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the downstream effector functions of APCs (to elicit an immune response) induced by a control polypeptide (having a corresponding wild type amino acid sequence).

In some cases, a high affinity BTN3A ectodomain polypeptide (a) induces an increase in the secretion of one or more helper cytokines (e.g., two or more, three or more, four or more, five or more helper cytokines) from APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the secretion of helper cytokines from APCs induced by a control polypeptide (having a corresponding wild type amino acid sequence); (b) induces an increase in the production of one or more costimulatory molecules (e.g., two or more, three or more, four or more, five or more costimulatory molecules) by APCs by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the production of costimulatory molecules induced by a control polypeptide (having a corresponding wild type amino acid sequence); and (c) induces an increase in the one or more downstream effector functions (e.g., two or more, three or more, four or more, five or more downstream effector functions) of APCs (to elicit an immune response) by 1.1-fold or more (e.g., 1.2-fold or more, 1.5-fold or more, 1.8-fold or more, 2-fold or more, 2.5-fold or more, 3-fold or more, 4-fold or more, 5-fold or more, or 10-fold or more) as compared to the downstream effector functions of APCs (to elicit an immune response) induced by a control polypeptide (having a corresponding wild type amino acid sequence).

In some cases, the above methods (e.g., generating and/or identifying a high affinity BTN3A ectodomain polypeptide) can include a step of mutating a nucleic acid encoding a BTN3A ectodomain polypeptide to generate a nucleic acid encoding a candidate high affinity BTN3A ectodomain polypeptide. Thus, such methods can include steps where by candidate agents are generated, and then tested. In some cases, such methods can include rounds (one one or more, two or more, three or more, four or more rounds, etc.) of generating a nucleic acid encoding a candidate high affinity BTN3A ectodomain polypeptide, followed by testing/assaying the candidate high affinity BTN3A ectodomain polypeptide (e.g., as described above), to determine if the candidate is in fact a high affinity BTN3A ectodomain polypeptide. Such rounds of mutation and selection (assay) can be iterative such that one does not need to stop after identifying a high affinity BTN3A ectodomain polypeptide, but instead one can continue rounds of mutation and selection (assays) to arrive at (i.e., generate) a high affinity BTN3A ectodomain polypeptide with even greater affinity.

Also within the scope of the disclosure are kits comprising the compositions (e.g., BTN3A ectodomain polypeptides and formulations thereof) of the disclosure and instructions for use. The kit can further contain an additional reagent, e.g., a reagent/component described herein, a diluent for reconstitution, etc. The components of a subject kit can be present in one or more containers (e.g., the same or separate containers). As an illustrative example, a subject kit can include, in addition to a subject BTN3A ectodomain polypeptide, or nucleic acid encoding a subject BTN3A ectodomain polypeptide, one or more of: a chemotherapeutic drug, an ADCC-inducing antibody (e.g., an opsonizing antibody, an anti-tumor antibody, and the like), an anti-infection drug (e.g., an anti-viral drug), etc. Kits typically include a label indicating the intended use of the contents of the kit. The term label includes any writing, or recorded material supplied on or with the kit, or which otherwise accompanies the kit.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-71 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of activating an antigen presenting cell (APC), comprising:

contacting an APC or a monocyte with a BTN3A ectodomain polypeptide that comprises a BTN3A ectodomain and lacks a BTN3A transmembrane domain, in an amount and for a period of time effect to activate the APC or to induce the monocyte to differentiate and mature into an activated APC.

2. The method according to 1, wherein the BTN3A ectodomain polypeptide comprises a BTN3A1 ectodomain.
3. The method according to 1, wherein the BTN3A ectodomain polypeptide comprises a BTN3A2 ectodomain.
4. The method according to 1, wherein the BTN3A ectodomain polypeptide comprises a BTN3A3 ectodomain.
5. The method according to 1, wherein the BTN3A ectodomain comprises an amino acid sequence having 80% or more sequence identity with the wild type BTN3A ectodomain amino acid sequence set forth in any of SEQ ID NOs: 10, 13, and 15.
6. The method according to any of 1-5, wherein the BTN3A ectodomain polypeptide comprises a dimerization moiety.
7. The method according to any of 1-5, wherein the BTN3A ectodomain polypeptide is a monomer.
8. The method according to any of 1-7, wherein the BTN3A ectodomain polypeptide comprises a BTN3A ectodomain and a fusion partner.
9. The method according to 8, wherein the fusion partner is part or whole of an Fc region.
10. The method according to 9, wherein the Fc region is a human IgG4 Fc region.
11. The method according to 8, wherein the BTN3A ectodomain polypeptide is a multispecific protein and the fusion partner comprises a region that specifically binds to a target molecule that is different from the target molecule bound by the BTN3A ectodomain.
12. The method according to 11, wherein the fusion partner comprises a region that specifically binds an antigen selected from: CTLA-4, Lag-3, BTLA, Tim-3, CD244, CD40, CD40L, CD47, SIRPα, PD-1, and PD-L1.
13. The method according to any of 1-12, wherein the BTN3A ectodomain polypeptide comprises a detectable label.
14. The method according to any of 1-13, wherein said contacting comprises administering the BTN3A ectodomain polypeptide to an individual with cancer and/or an infectious disease.
15. The method according to 14, wherein the BTN3A ectodomain polypeptide is co-administered with an ADCC-inducing antibody.
16. The method according to 15, wherein the ADCC-inducing antibody specifically binds to a tumor antigen.
17. The method according to 16, wherein the tumor antigen is selected from: CD20, CD52, CD38, HER-2, 17-1A, and EGFR.
18. The method according to any of 1-13, wherein said contacting is in vitro or ex vivo.
19. The method according to 18, wherein the method comprises contacting the APC or monocyte with a tumor antigen.
20. The method according to 19, wherein the method comprises contacting the APC or monocyte with a tumor lystate.
21. The method according to 19 or 20, wherein the APC or monocyte is contacted with the tumor antigen and/or the tumor lysate in the presence of the BTN3A ectodomain polypeptide.
22. The method according to 19 or 20, wherein the APC or monocyte is contacted with the tumor antigen and/or tumor lysate prior to or after said contacting with the BTN3A ectodomain polypeptide.
23. The method according to any of 18-22, wherein the activated APC is introduced into an individual with cancer and/or an infectious disease.
24. The method according to 23, wherein the activated APC is autologous to the individual.
25. The method according to any of 1-24, wherein the activated APC is used to cross-prime a naive T cell into an antigen specific effector cell.
26. The method according to 25, wherein the activated APC is contacted in vitro or ex vivo with the naive T cell.
27. The method according to 25 or 26, wherein the antigen specific effector cell is introduced into an individual with cancer and/or an infectious disease.
28. The method according to any of 25-27, wherein the naive T cell is autologous to the individual.
29. A pharmaceutical BTN3A ectodomain composition, comprising:

(a) a BTN3A ectodomain polypeptide comprising a BTN3A ectodomain and lacking a BTN3A transmembrane domain; and

(b) a pharmaceutical excipient,

wherein the composition is a unit dose formulation that is effective to activate antigen presenting cells (APCs) in an individual.

30. The composition according to 29, wherein the BTN3A ectodomain polypeptide comprises a BTN3A1 ectodomain.
31. The composition according to 29, wherein the BTN3A ectodomain polypeptide comprises a BTN3A2 ectodomain.
32. The composition according to 29, wherein the BTN3A ectodomain polypeptide comprises a BTN3A3 ectodomain.
33. The composition according to any of 29-32, wherein the BTN3A ectodomain comprises an amino acid sequence having 80% or more sequence identity with the amino acid sequence set forth in any of SEQ ID NOs: 10, 13, and 15.
34. The composition according to any of 29-33, wherein the BTN3A ectodomain polypeptide comprises a dimerization moiety.
35. The composition according to 34, wherein the dimerization moiety comprises an amino acid sequence having 80% or more sequence identity with the amino acid sequence set forth in any of SEQ ID NOs: 31-34.
36. The composition according to any of 29-33, wherein the BTN3A ectodomain polypeptide is a monomer.
37. The composition according to any of 29-36, wherein the BTN3A ectodomain polypeptide comprises a BTN3A ectodomain and a fusion partner.
38. The composition according to 37, wherein the fusion partner is part or whole of an Fc region.
39. The composition according to 38, wherein the Fc region is a human IgG4 Fc region.
40. The composition according to 39, wherein the BTN3A ectodomain polypeptide comprises an amino acid sequence having 80% or more sequence identity with the amino acid sequence set forth in SEQ ID NO: 30.
41. The composition according to 37, wherein the BTN3A ectodomain polypeptide is a multispecific protein and the fusion partner comprises a region that specifically binds to a target molecule that is different from the target molecule bound by the BTN3A ectodomain.
42. The composition according to 41, wherein the fusion partner comprises a region that specifically binds to a tumor antigen.
43. The composition according to 41, wherein the fusion partner comprises a region that specifically binds an antigen selected from: CTLA-4, Lag-3, BTLA, Tim-3, CD244, CD40, CD40L, CD47, SIRPα, PD-1, and PD-L1.
44. The composition according to any of 29-43, wherein the BTN3A ectodomain polypeptide comprises a detectable label.
45. The composition according to any of 29-44, further comprising an ADCC-inducing antibody.
46. The composition according to 45, wherein the ADCC-inducing antibody specifically binds to a tumor antigen.
47. The composition according to 45, wherein the ADCC-inducing antibody specifically binds to an antigen selected from: CD20, CD52, CD38, HER-2, 17-1A, and EGFR.
48. A method of treating an individual having cancer and/or having a chronic infection, the method comprising:

administering to the individual, a pharmaceutical BTN3A ectodomain composition according to any of 29-47, in an amount effective to reduce the number of cancer cells and/or infected cells in the individual.

49. The method according to 48, wherein the individual is a human.
50. The method according to 48 or 49, wherein the method comprises co-administering the pharmaceutical BTN3A ectodomain composition with an ADCC-inducing antibody.
51. The method according to 50, wherein the ADCC-inducing antibody specifically binds to a tumor antigen.
52. The method according to 51, wherein the ADCC-inducing antibody specifically binds to an antigen selected from: CD20, CD52, CD38, HER-2, 17-1A, and EGFR.
53. The method according to any of 50-52, wherein the pharmaceutical BTN3A ectodomain composition and the ADCC-inducing antibody are not administered simultaneously.
54. The method according to any of 50-52, wherein the pharmaceutical BTN3A ectodomain composition and the ADCC-inducing antibody are administered simultaneously.
55. A BTN3A ectodomain polypeptide, or a nucleic acid encoding said BTN3A ectodomain polypeptide, wherein the BTN3A ectodomain polypeptide comprises a BTN3A ectodomain and a dimerization moiety, and lacks a BTN3A transmembrane domain.
56. The BTN3A ectodomain polypeptide, or nucleic acid encoding said BTN3A ectodomain polypeptide, according to 55, wherein the BTN3A ectodomain polypeptide comprises a BTN3A1 ectodomain.
57. The BTN3A ectodomain polypeptide, or nucleic acid encoding said BTN3A ectodomain polypeptide, according to 55, wherein the BTN3A ectodomain polypeptide comprises a BTN3A2 ectodomain.
58. The BTN3A ectodomain polypeptide, or nucleic acid encoding said BTN3A ectodomain polypeptide, according to 55, wherein the BTN3A ectodomain polypeptide comprises a BTN3A3 ectodomain.
59. The BTN3A ectodomain polypeptide, or nucleic acid encoding said BTN3A ectodomain polypeptide, according to 55, wherein the BTN3A ectodomain comprises an amino acid sequence having 80% or more sequence identity with the wild type BTN3A ectodomain amino acid sequence set forth in any of SEQ ID NOs: 10, 13, and 15.
60. A BTN3A ectodomain polypeptide, or a nucleic acid encoding said BTN3A ectodomain polypeptide, wherein the BTN3A ectodomain polypeptide comprises a BTN3A ectodomain and a human IgG4 Fc region, and lacks a BTN3A transmembrane domain.
61. The BTN3A ectodomain polypeptide, or nucleic acid encoding said BTN3A ectodomain polypeptide, according to 60, wherein the BTN3A ectodomain polypeptide comprises a BTN3A1 ectodomain.
62. The BTN3A ectodomain polypeptide, or nucleic acid encoding said BTN3A ectodomain polypeptide, according to 60, wherein the BTN3A ectodomain polypeptide comprises a BTN3A2 ectodomain.
63. The BTN3A ectodomain polypeptide, or nucleic acid encoding said BTN3A ectodomain polypeptide, according to 60, wherein the BTN3A ectodomain polypeptide comprises a BTN3A3 ectodomain.
64. The BTN3A ectodomain polypeptide, or nucleic acid encoding said BTN3A ectodomain polypeptide, according to 60, wherein the BTN3A ectodomain comprises an amino acid sequence having 80% or more sequence identity with the wild type BTN3A ectodomain amino acid sequence set forth in any of SEQ ID NOs: 10, 13, and 15.
65. A method for enhancing immune responses to an antigenic compound, comprising:

administering to an individual: (a) a BTN3A ectodomain polypeptide comprising a BTN3A ectodomain and lacking a BTN3A transmembrane domain; and (b) an antigen.

66. The method according to 65, wherein the source of the antigen is selected from: a human, a non-human animal, a plant, a bacterial cell, an archaeal cell, a fungus, a virus, a parasite, and a cancer cell.
67. The method according to 65 or 66, wherein the individual is a mammal.
68. The method according to 67, wherein the individual is a human.
69. The method according to any of 65-68, wherein the antigen is a vaccine.
70. The method according to 69, wherein the vaccine is directed at Tuberculosis, Malaria, Human Immunodeficiency Virus (HIV), RotaVirus, Herpes Simplex Virus (HSV), or Cytomegalovirus (CMV).
71. The method according to 70, wherein the vaccine is a cancer vaccine.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

The following examples demonstrate the creation of BTN3A ectodomain polypeptides that effectively activate APCs. The BTN3A ectodomain polypeptides can be used as therapeutics for a variety of indications, including cancer and infectious disease.

### Example 1

The current model for T-cell activation postulates that naive T-cells require two signals for full activation: (i) a signal provided through the binding of processed antigens presented to the T-cell receptor by major histocompatibility complex (MHC) class I molecules; and (ii) an additional signal provided by the interaction of co-stimulatory molecules on the surface of T-cells and their ligands on antigen presenting cells. Recognition of an antigen by a naive T-cell is insufficient in itself to trigger T-cell activation. Without a co-stimulatory signal, T-cells may be eliminated either by death or by induction of anergy.

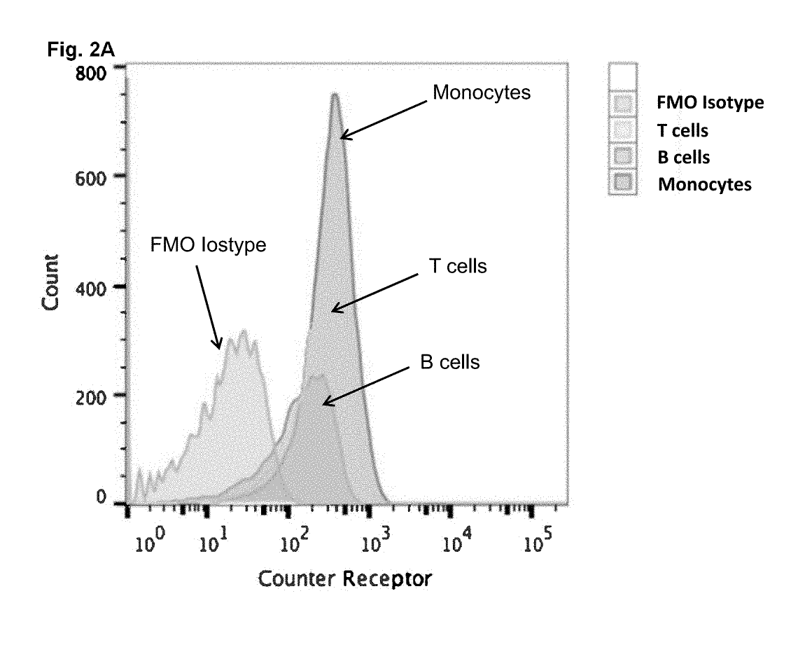

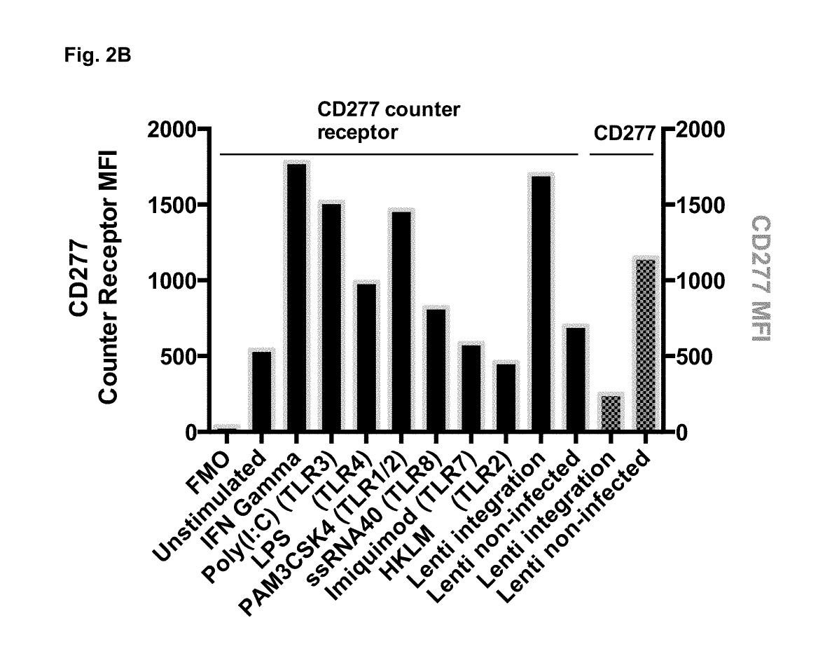

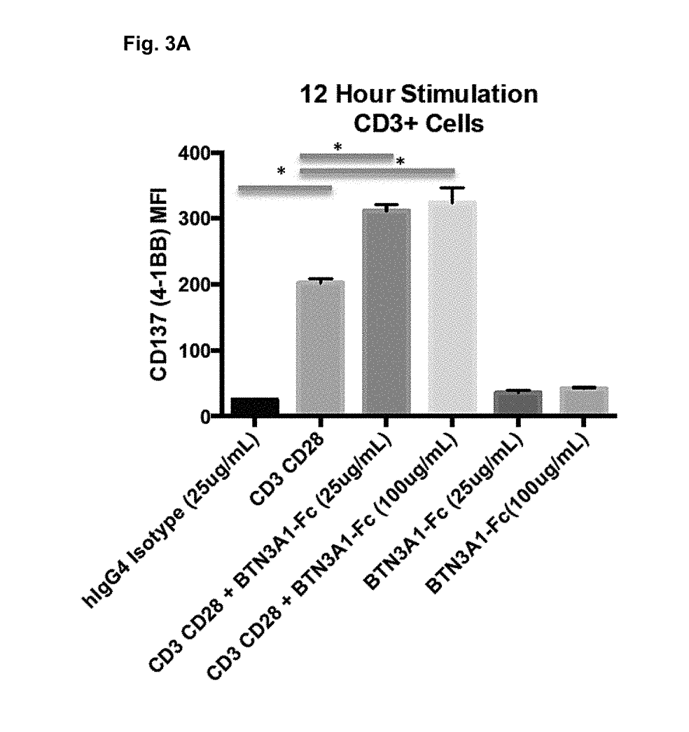

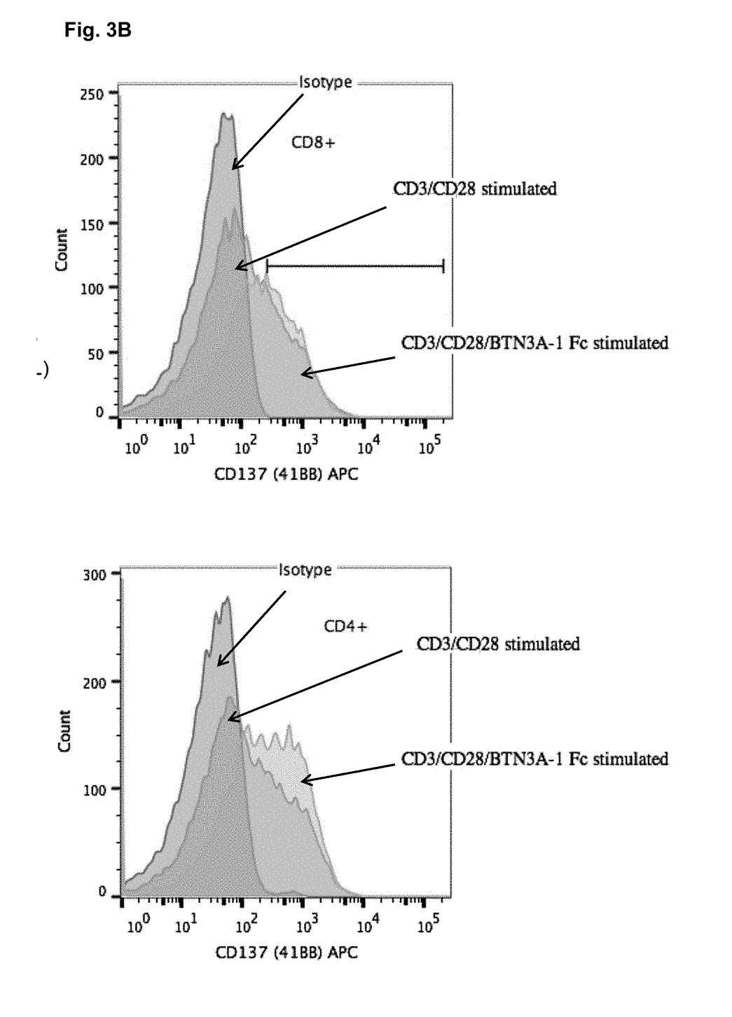

Butyrophilin 3A1 (BTN3A1; also known as BTN3A1) is a member of the Butrophyllin family of molecules. Using fluorescent, recombinant BTN3A1 protein (a detectably labeled BTN3A1 ectodomain polypeptide), broad staining was observed across multiple subsets of peripheral blood (e.g., the presence of a counter-receptor for BTN3A1 on resting T, B, and NK (Natural Killer) cells was observed) (FIG. 2A). In addition, Histogram plots demonstrate exposure to γ (gamma) interferon, TLR ligands, and virus stimulation upregulate CD277 counter-receptor expression (FIG. 2B). Furthermore, administration of recombinant BTN3A1 augmented TCR-mediated T cell activation (FIG. 3A-3B). These results indicate that BTN3A1-Fc enhances CD3/CD28 mediated activation. These results indicated a possible role for BTN3A1 as a co-stimulatory molecule.

### Example 2

### BTN3A1 Ectodomain Polypeptides Induce Maturation and Activation of Antigen Presenting Cells to Induce Downstream T Cell Responses

### BTN3A1 Ectodomain Polypeptides Induce Activation and Maturation of Dendritic Cells (DCs) and Monocyte Populations

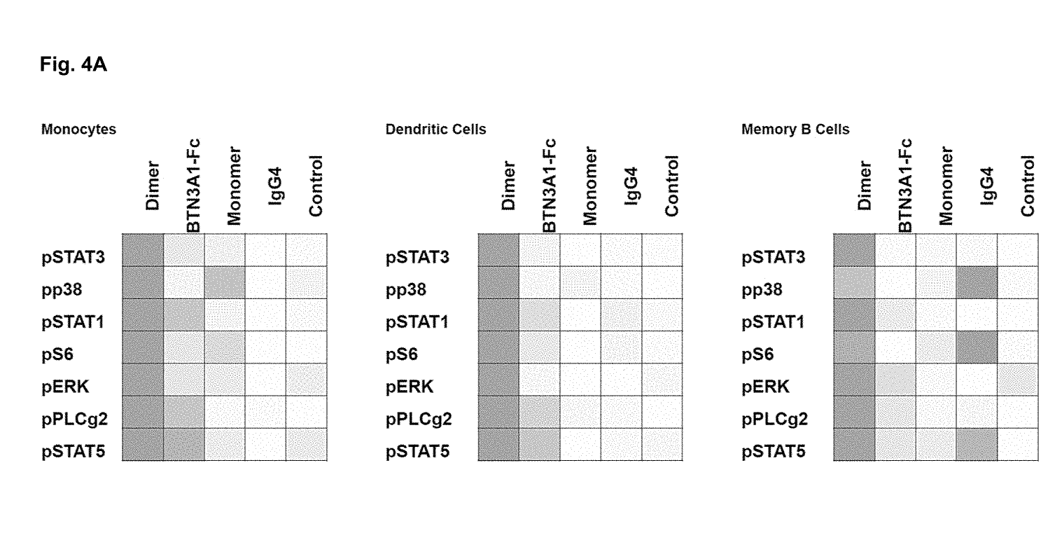

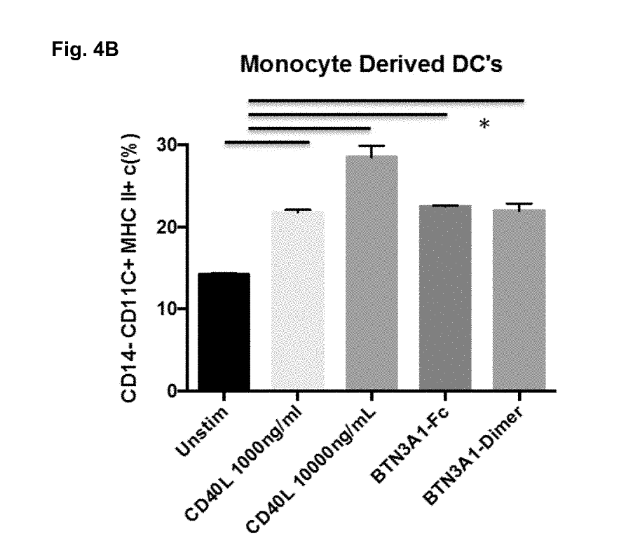

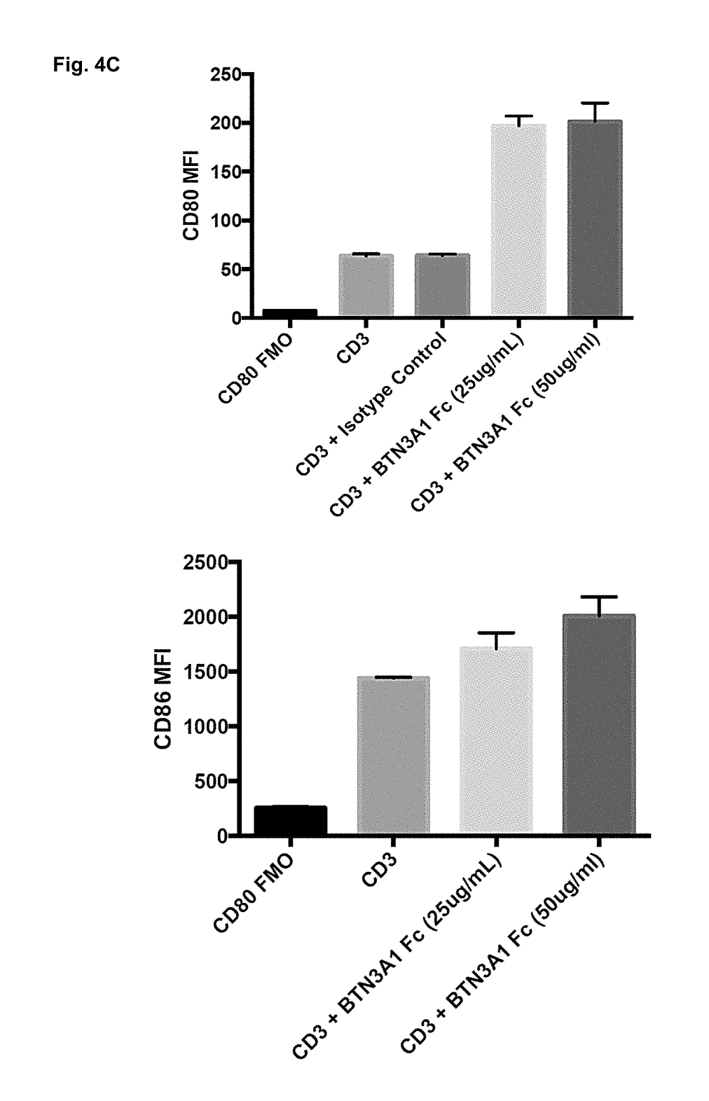

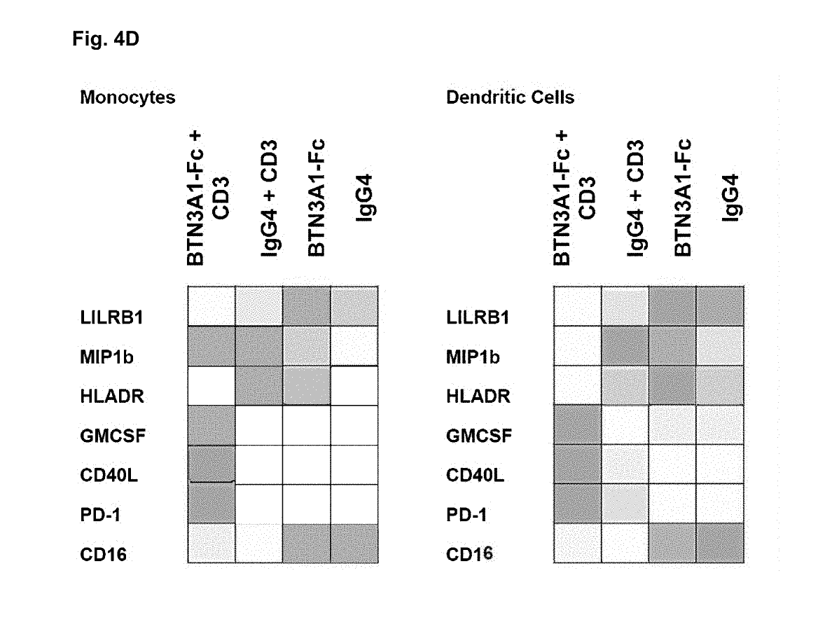

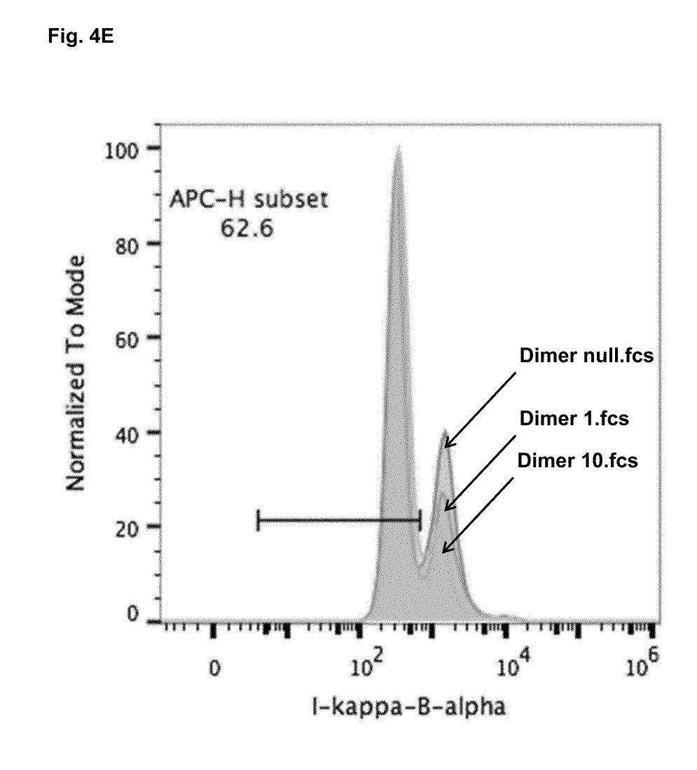

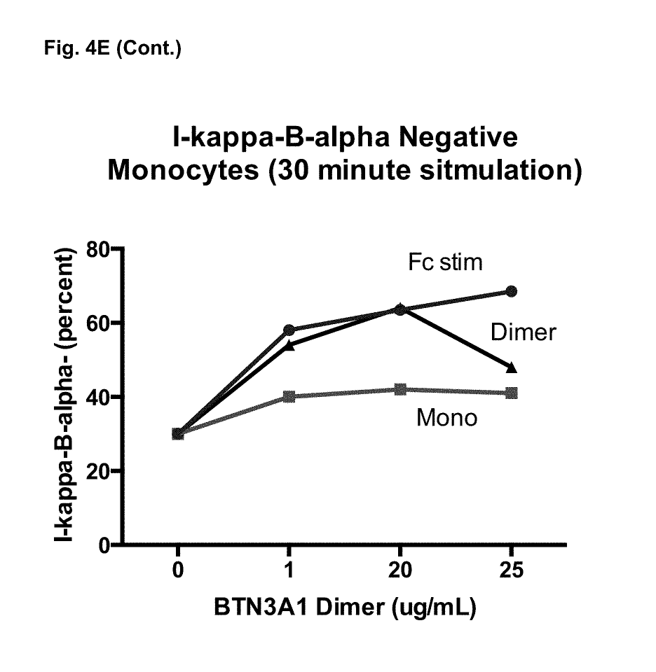

BTN3A1 recombinant proteins (BTN3A1 ectodomain polypeptides) induced rapid signaling changes within antigen presenting populations (dendritic cells, monocytes, B cells, and γδ T cells) (FIG. 4A). In dendritic cells, for example, 30 minutes after administration of 40 μg/ml of a dimeric form of BTN3A1 ectodomain, significant increases were observed in downstream Akt kinase activation, STAT1, STAT5, and ERK1/2 signaling. These signaling changes have been associated with maturation, expansion, and activation of immature monocytes into professional antigen presenting cells (APCs). BTN3A1 proteins promoted differentiation of Dendritic Cells from monocytes. Purified monocytes were plated in standard culture media (RPMI+5% Human Serum) and stimulated with BTN3A1-Fc or Dimer proteins for 72 hours. The percentage of CD14-CD11C+MHCII+ cells were quantified by FACs from the bulk culture (FIG. 4B). The administration of an Fc-fusion with a BTN3A1 ectodomain (“BTN3A1-Fc”) promoted dendritic cell and monocyte up-regulation of molecules requisite for stimulation of T cells (e.g., upregulation of CD80, CD86, and CD40L) (FIG. 4C and FIG. 4D). Moreover, engagement of BTN3A1-Fc on monocytes and dendritic cells resulted in loss of surface expression of the inhibitory cell surface receptors LILRB1 and PD1. The disinhibition of these APC populations was enhanced when used in combination with T cell receptor engagement using CD3 antibody or conjugated CD3+CD28 beads. Additionally, BTN3A1 dimer proteins were shown here to signal through NF-kappa-B complexes. Purified monocytes were stimulated with BTN3A1 dimer proteins at indicated concentrations and stained for the intracellular inhibitor of dimeric NF-kappa-B (I-kappa-B-alpha). Histogram plots indicate rapid degradation of I-kappa-B-alpha, an NF-kappa-B inhibitor, 30 minutes after stimulation with BTN3A1 proteins (FIG. 4E).

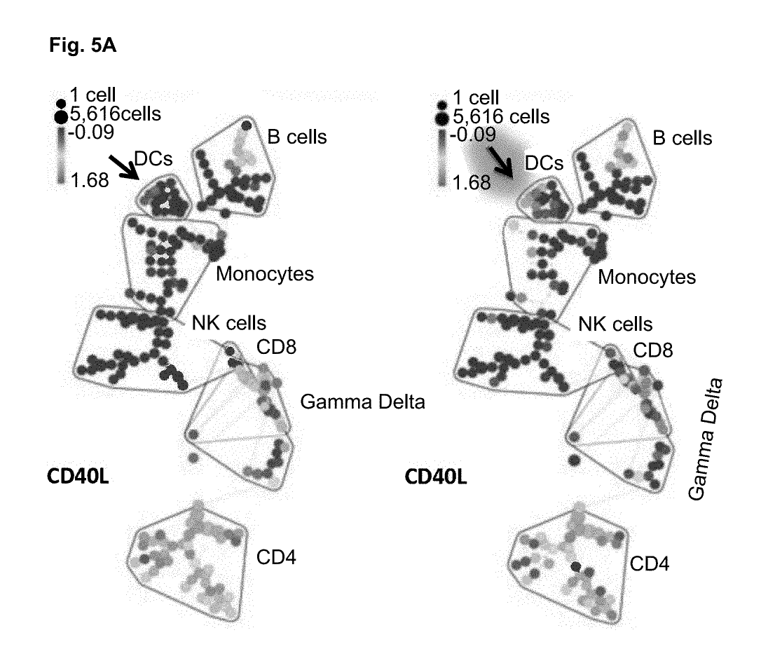

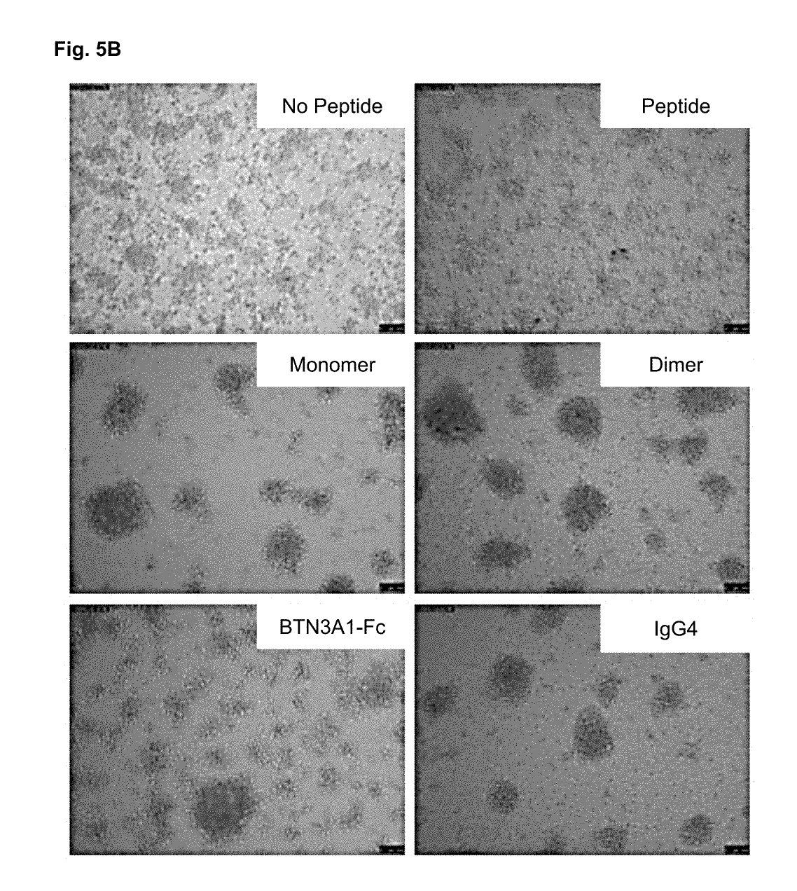

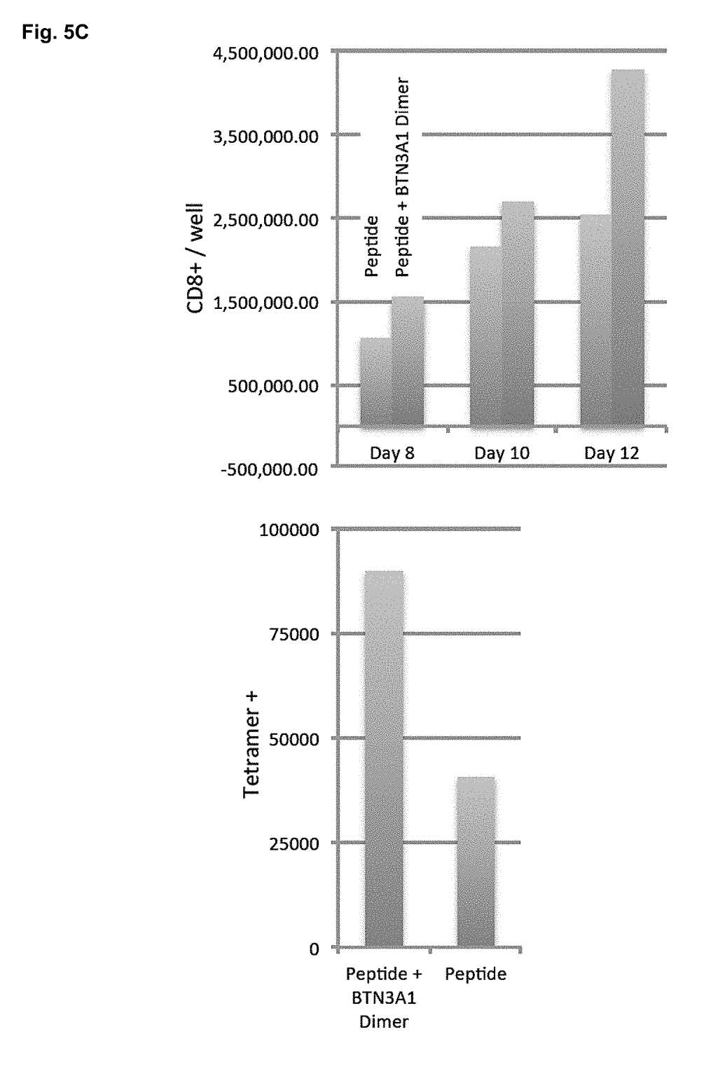

To test for the ability of BTN3A1 ectodomain polypeptides to enhance antigen presentation, BTN3A1 ectodomain polypeptides (monomer, dimer, and BTN3A1-Fc) were administered to immature monocyte derived dendritic cell cultures after 72 hours in media supplemented with IL4 and GMCSF. Matured dendritic cells were pulsed with Melan-A peptide and subsequently co-cultured with naïve CD8 T cells (FIG. 5B and FIG. 5C, also see FIG. 5A). MHC multimer staining for tumor antigen was assessed on day 10. Samples that received BTN3A1 ectodomain polypeptides showed a significant increase in the absolute quantity of CD8+ and CD8+ tetramer+ T cells. Moreover, characteristic morphologic changes of activated dendritic cells were observed within these samples, with large clusters of T cells in close apposition to spindle shaped APCs. Finally, the enhancement in T cell stimulatory capacity in the Butryrophilin groups appeared to be greatest when using the Dimer form of the BTN3A1 ectodomain. These findings suggest an Fc receptor independent activation of both monocytes and dendritic cells.

BTN3A1 Ectodomain Polypeptides Induce Differentiation of Monocytes into Inflammatory Monocytes with Antigen Presentation Properties

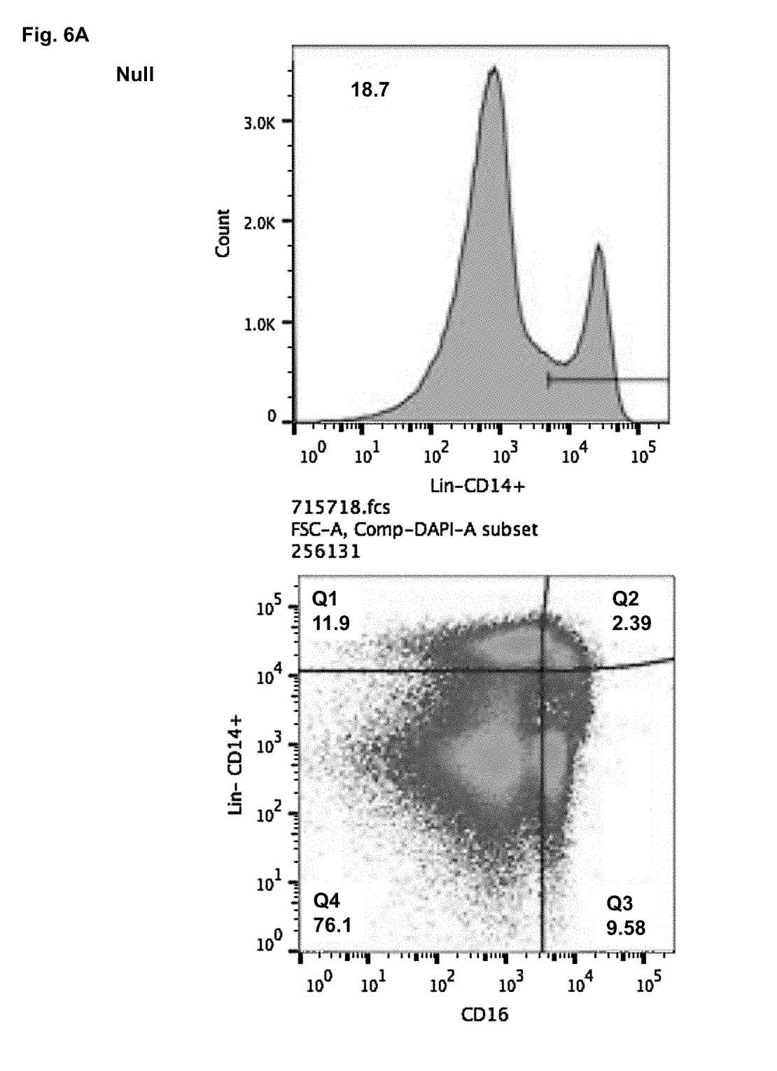

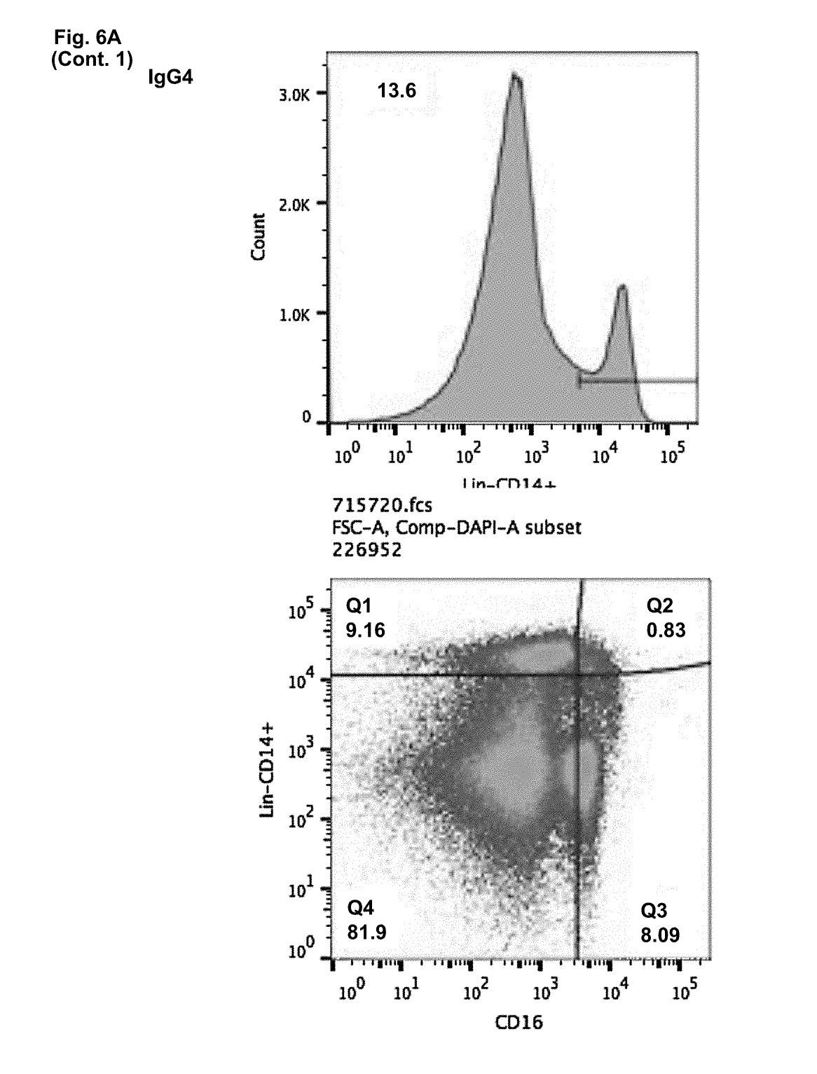

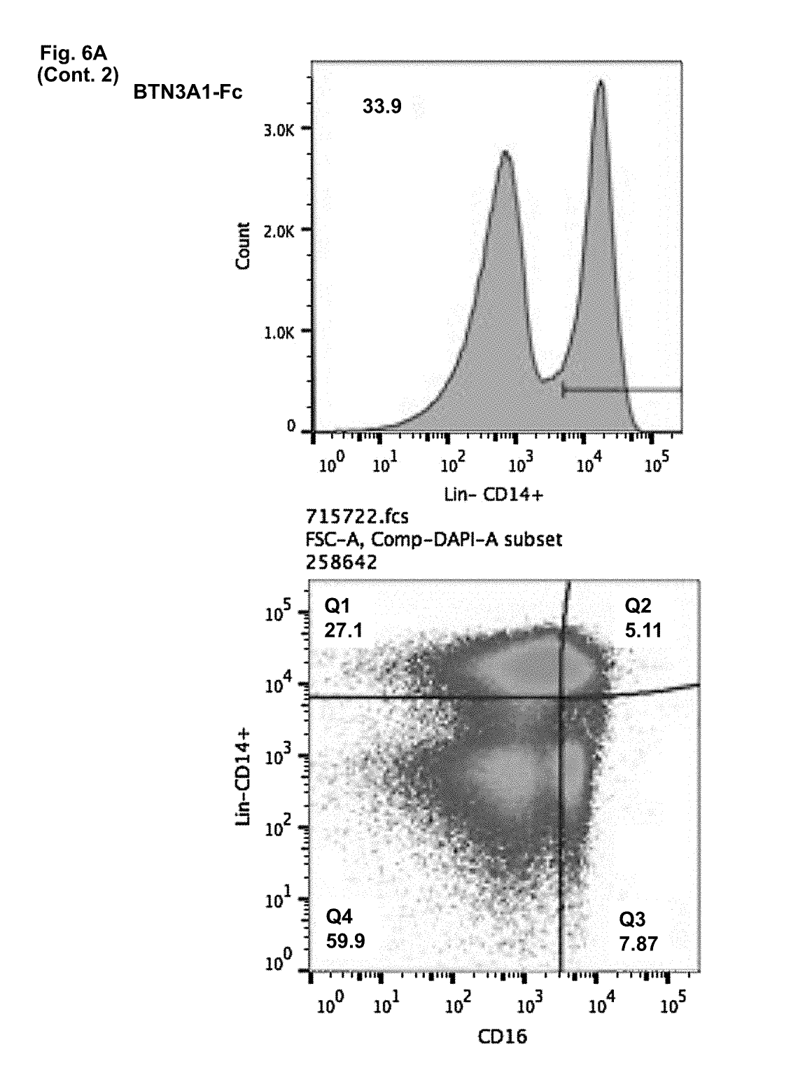

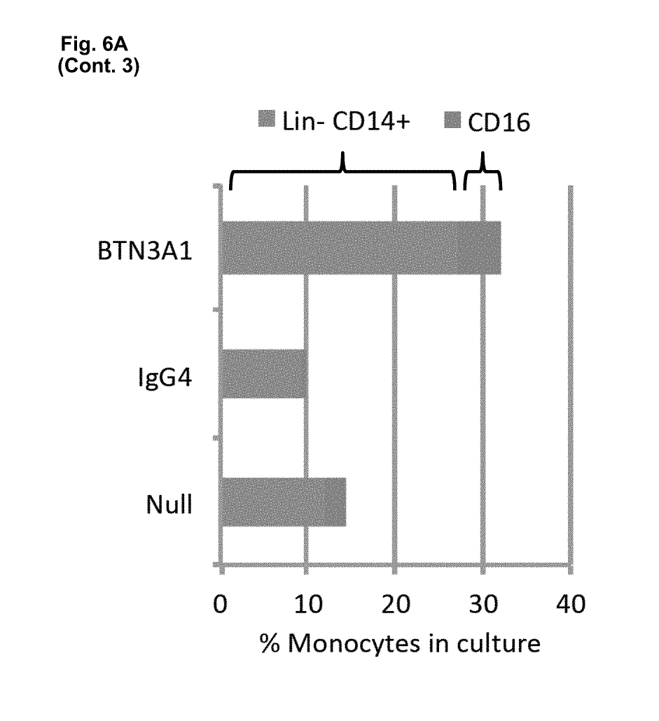

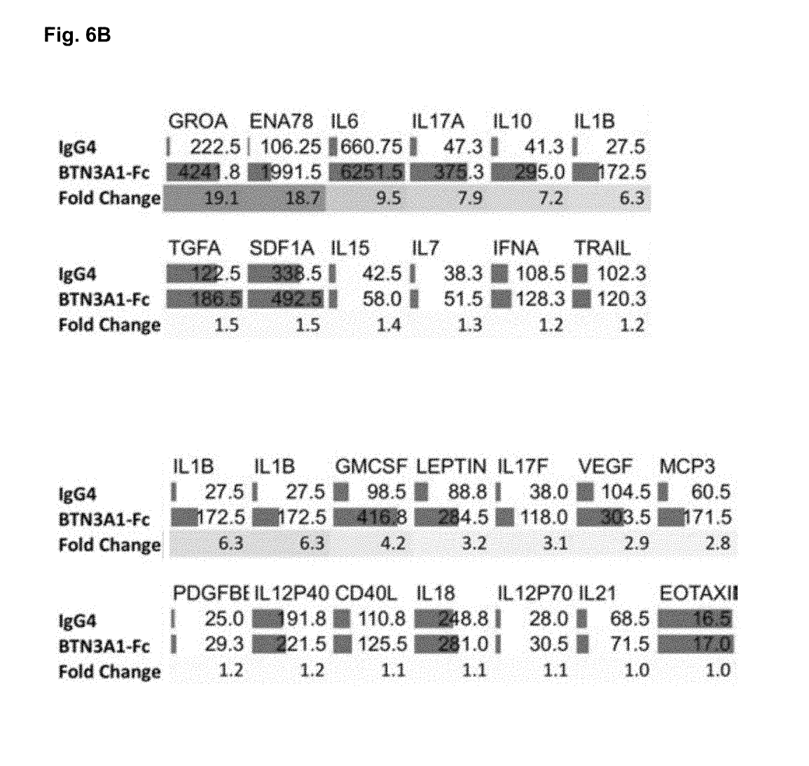

Upon administration of BTN3A1 ectodomain polypeptides to PBMC's, a rapid proliferation of lineage negative (CD3−, CD56−, CD19−, CD20−) CD14+ peripheral blood monocytes was observed (FIG. 6A, compare “BTN3A1-Fc” to “Null” and “IgG4”). The expansion in monocytes was accompanied by a polarization to an inflammatory phenotype; characterized by increases in expression of CD16+, HLA.-DR, CD80, and CD86. These changes were accompanied by secretion of monocyte derived cytokines into culture media known to initiate early activation of CD8+ T cells, naïve and memory B cells, NK cells, and monocyte chemo attractants (FIG. 6B). Significant increases in GM-CSF levels were also observed upon administration of BTN3A1 ectodomain polypeptides. In accordance with this, purified monocyte populations pulsed with tumor-associated antigen after priming with BTN3A1 protein expanded antigen-specific cytotoxic T lymphocytes (FIG. 5B and FIG. 5C).

### BTN3A1 Ectodomain Polypeptides the Antigen Presenting Function of B Cells

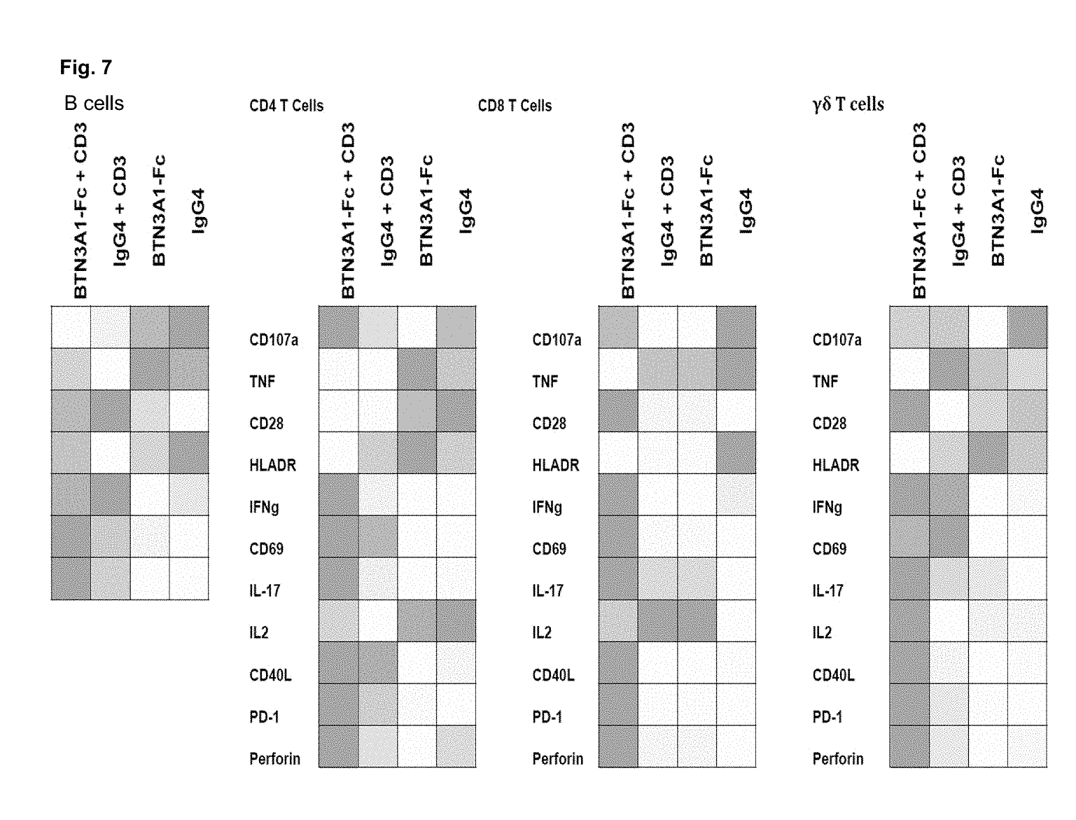

Though infrequent, B cells can activate or tolerize T cells and thus participate in the generation or regulation of immune responses. These responses have shown to be dependent on help from activated CD4 T cells. A role for BTN3A1 ectodomain polypeptides activating the antigen presenting function of B cells is indicated by the data presented herein. After encountering BTN3A1 ectodomain polypeptides, B cells increased expression of CD40L, HLA−. DR, and CD86, the molecule that binds to CD28 (FIG. 7). On the T cell side, significantly enhanced CD28 expression was observed upon BTN3A1 ectodomain polypeptides. In agreement with this, downstream Akt kinase activation, STAT1, STAT5, and ERK1/2 were activated in B cells upon administration of BTN3A1 ectodomain polypeptides. These pathways have been implicated downstream of B cell receptor signaling and in B cell development, and expansion. The most significant changes were observed when using the Dimer form of the BTN3A1 ectodomain.

### BTN3A1 Ectodomain Polypeptides Enhance Antibody Dependent Cell Mediated Cytotoxicity (ADCC)

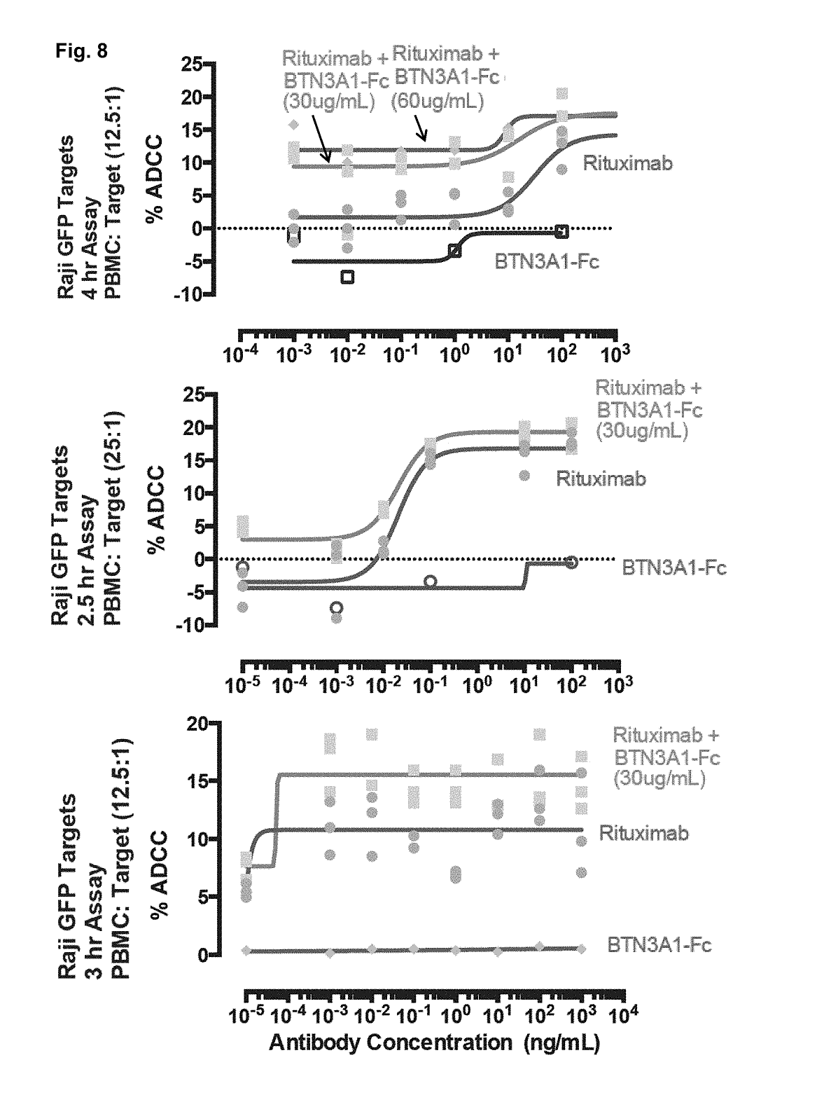

Enhanced killing of lymphoma (RAJI) target cells was observed upon administration of BTN3A1 ectodomain polypeptides in combination with Rituximab, an antibody that induces Antibody Dependent Cell Mediated Cytotoxicity (ADCC) of CD20-expressing cancer cells. These experiments were carried out using the indicated ratio of PBMCs to target cells (FIG. 8).

Enhanced ADCC was also observed upon administration of BTN3A1 ectodomain polypeptides when using in vitro derived cultures from purified γδ T cells as effectors cells. γδ T cells were expanded from fresh PBMC's to >95% purity, and ADCC assays were performed at a 5:1 effector: target ratio. These data are supported by multi-parameter mass cytometry staining that revealed induction of perforin and associated CD8 and γδ T cell activation markers (CD28, 41BB, CD69, CD25, and CD40L) upon administration of a BTN3A1 ectodomain polypeptide (FIG. 7).

### Use of BTN3A1 Ectodomain Polypeptides to Enhance Anti-Pathogen Responses

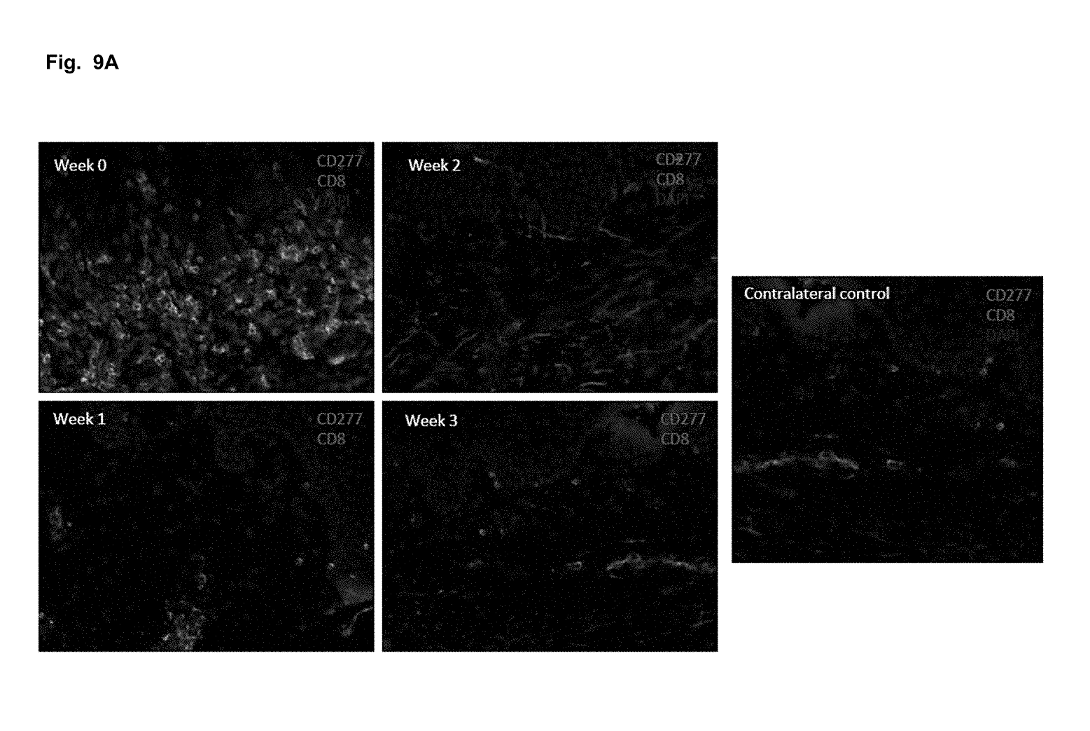

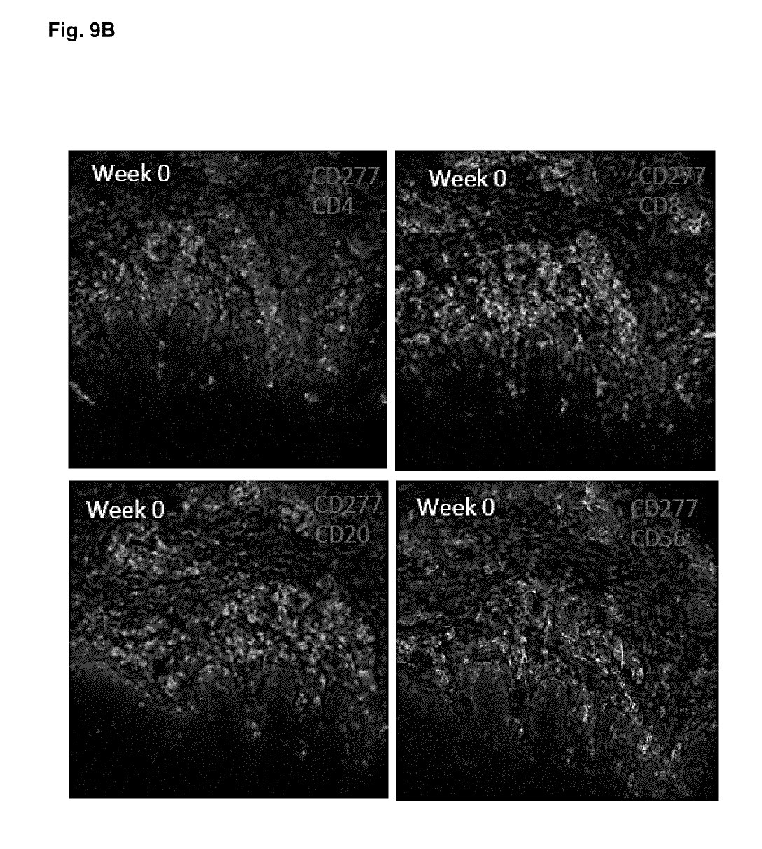

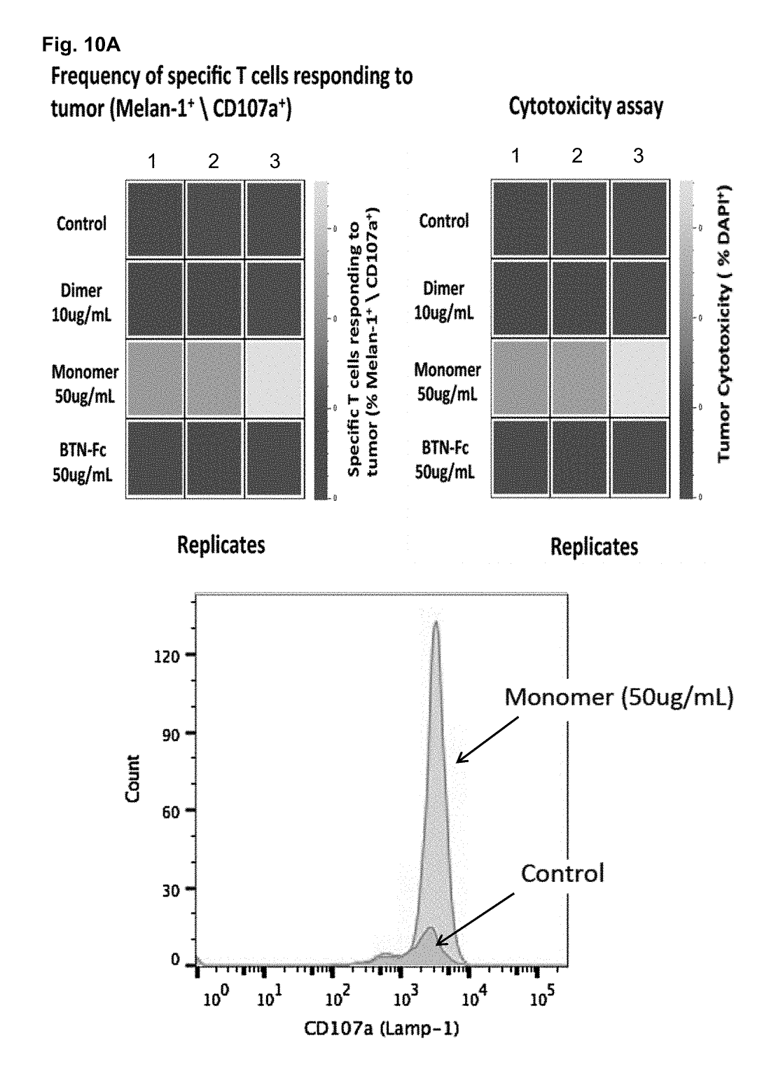

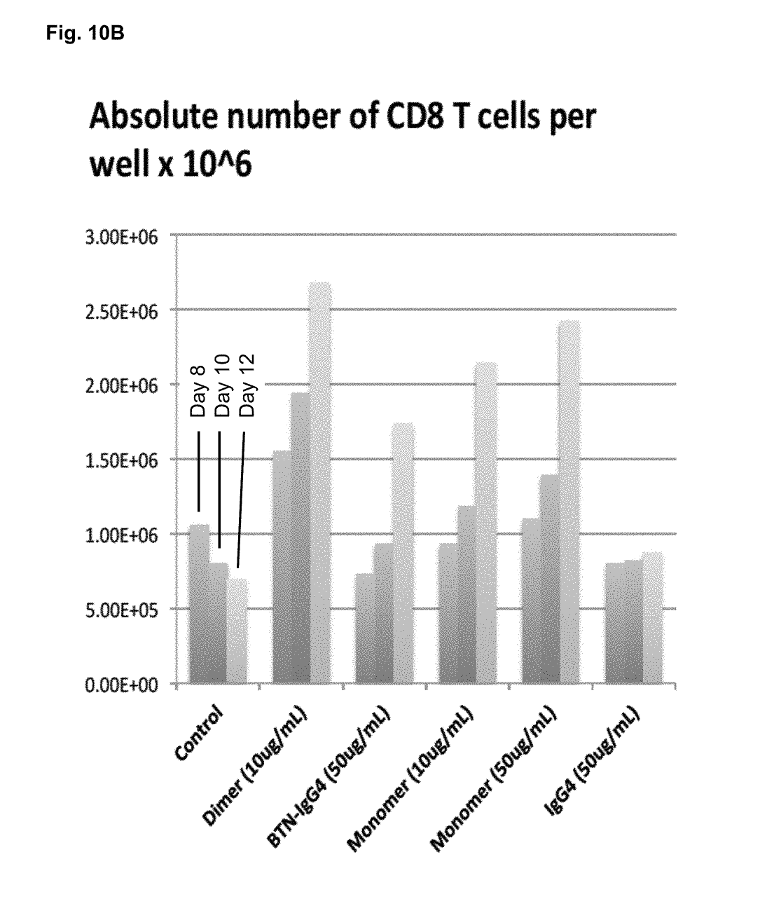

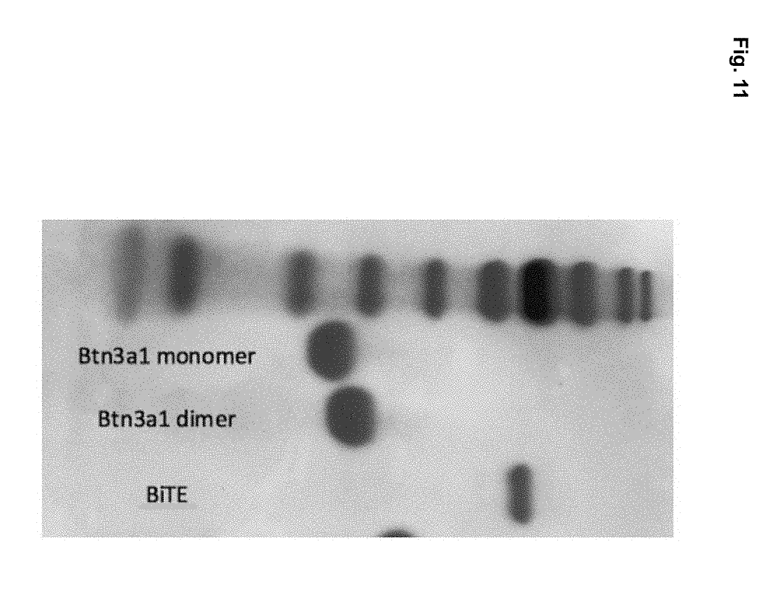

These data suggested an important role for BTN3A1 (BTN3A1) engagement on APCs and innate effector cells to augment anti-tumor immune responses. To investigate, BTN3A1 (BTN3A1) in anti-pathogen immunity tissue biopsy specimens from individuals with acute viral infections were stained. Subjects underwent serial biopsies in the setting of an acute viral skin eruption (HSV-II) and weekly thereafter and stained for leukocyte markers to gain insight into the spatial and temporal distribution of BTN3A1 expressing cells (FIG. 9). As a control, tissue biopsies were obtained in the contralateral limb. In control samples, BTN3A1 staining (using antibody BT3.1) was confined to rare staining CD8 T cells at the dermal:epithelial junction. A massive upregulation of BTN3A1 staining was observed at the site of viral infection, however, with enhanced staining in CD8+ T cells and surrounding keritnocytes. Weekly survellience biopsies of these injured sights revealed gradual diminution in BTN3A1 surface staining as the wound healed. These findings support a role for BTN3A1 immune responses in acute viral infection and suggest promoting BTN3A1 immune responses using BTN3A1 ectodomain polypeptides as an anti-pathogen immunotherapeutic strategy.

### Example 3

### Identification of CD277 Candidate Binding Partners

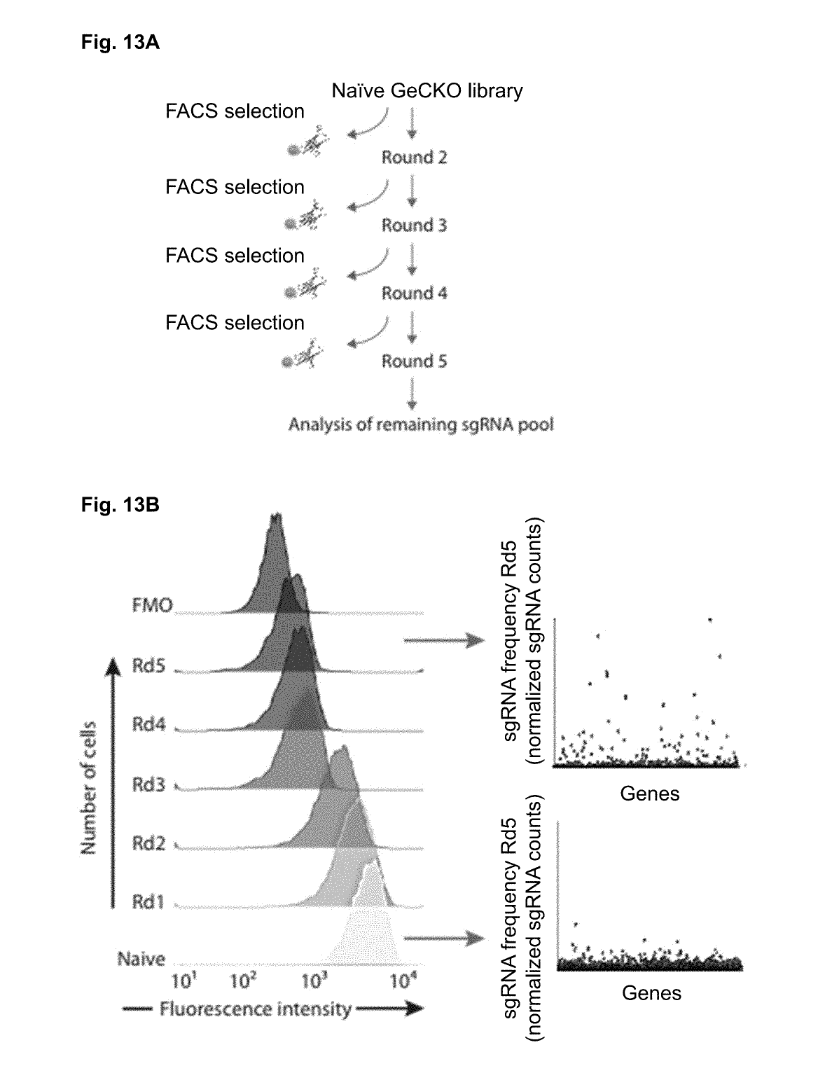

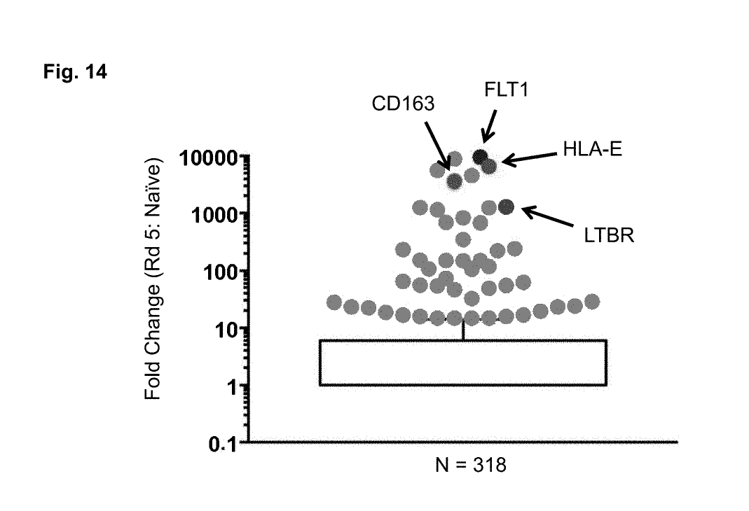

Initial studies using a BTN3A1 ectodomain fused to IgG1 detected high levels of binding on a number of cell lines, suggesting the existence of a counter-receptor (Compte et al, European journal of immunology 34, 2089, August, 2004). To pinpoint gene targets involved in BTN3A binding, a fluorescently coupled BTN3A ectodomain fused to the Fc fragment of human IgG4 was utilized to conduct a FACs-based selection screen from a pooled lentiCRISPR-Cas9 knockout library (GeCKO)(Sanjana et al., Nature methods 11, 783, August, 2014) (FIG. 13A-13B). By selecting cells that were rendered non-binding by CAS9:sgRNA-mediated modification, sgRNAs were identified that ablated BTN3A binding. To assess the feasibility of this approach, cell lines were screened for binding affinity to BTN3A-IgG4; the Natural killer cell line YT-1 showed near uniformly positive staining and was therefore transduced with the GeCKO library. Following puromycin selection, cells were FACs sorted for non-binding to CD277 (BTN3A1), expanded for 7 days in bulk culture, and reanalyzed. The library was subjected to 8 rounds of selection then harvested for amplicon deep sequencing (FIG. 13A-13B). The selection strategy resulted in a significant reduction in library diversity compared to round 0. The overall sgRNA pool by the end of the screen was restricted to a few (n=304), highly abundant sgRNAs. Ranking genes by log 10 fold change yielded several cell surface receptor candidates not previously implicated in CD277 (BTN3A1) biology, including the TNF superfamily member LTβR (sometimes referred to herein as LTBR) (3.69 Log 10 fold change), the VEGF receptor FLT1 (3.70 Log 10 fold change), the non-classical MHC molecule HLA-E (3.66 Log 10 fold change), the macrophage and γδ T cell scavenger receptor CD163 (3.56 Log 10 fold change), and the Wnt receptor receptor tyrosine kinase ROR2 (3.54 Log 10 fold change) (FIG. 14).

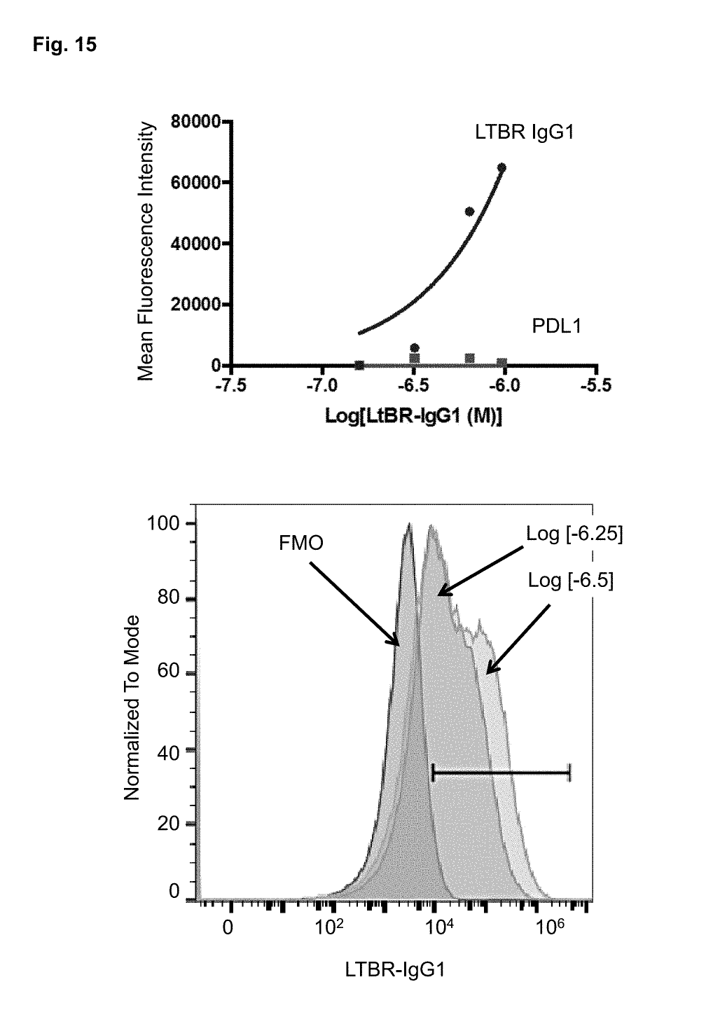

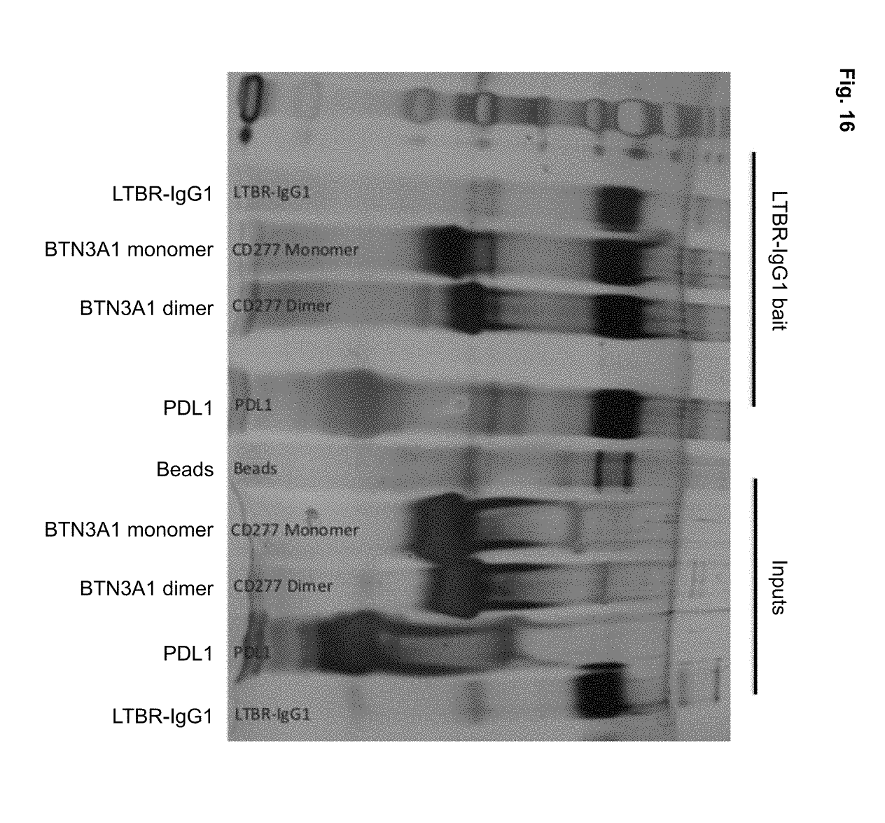

Using independent methods, binding between LTβR and the extracellular domain of BTN3A1 was confirmed. Yeast cells displaying the immunoglobulin constant domain (IgC), N-terminal immunoglobulin variable domain (IgV), and full-length BTN3A1 ectodomain were stained with purified LTβR-IgG1 fusion protein. As a control, we expressed the closest CD277 structural homologue, PDL1. BTN3A1 induced yeast specifically bound to LtβR-IgG1 (FIG. 15). To further investigate the potential interaction between LTβR and CD277 (BTN3A1), a pull-down assay was performed using purified LtβR-IgG1 as bait protein and recombinant BTN3A1 monomers and dimers as ligands. Protein complexes were captured on Protein A agarose affinity beads, washed in PBS, and eluted in SDS-PAGE sample buffer for downstream electrophoresis analysis. CD277 (BTN3A1) monomer and dimers specifically bound to LtβR-IgG1 (FIG. 16).

That which is claimed is:

1. A method of activating an antigen presenting cell (APC), comprising:
contacting an APC or a monocyte with a BTN3A ectodomain polypeptide that comprises a BTN3A ectodomain and lacks a BTN3A transmembrane domain, in an amount and for a period of time effect to activate the APC or to induce the monocyte to differentiate and mature into an activated APC.

2. The method according to claim 1, wherein the BTN3A ectodomain polypeptide comprises a BTN3A1 ectodomain.

3. The method according to claim 1, wherein the BTN3A ectodomain polypeptide comprises a BTN3A2 ectodomain.

4. The method according to claim 1, wherein the BTN3A ectodomain polypeptide comprises a BTN3A3 ectodomain.

5. The method according to claim 1, wherein the BTN3A ectodomain comprises an amino acid sequence having 80% or more sequence identity with the wild type BTN3A ectodomain amino acid sequence set forth in any of SEQ ID NOs: 10, 13, and 15.

6. The method according to any of claims 1-5, wherein the BTN3A ectodomain polypeptide comprises a dimerization moiety.

7. The method according to any of claims 1-5, wherein the BTN3A ectodomain polypeptide is a monomer.

8. The method according to any of claims 1-7, wherein the BTN3A ectodomain polypeptide comprises a BTN3A ectodomain and a fusion partner.

9. The method according to claim 8, wherein the fusion partner is part or whole of an Fc region.

10. The method according to claim 9, wherein the Fc region is a human IgG4 Fc region.

11. The method according to claim 8, wherein the BTN3A ectodomain polypeptide is a multispecific protein and the fusion partner comprises a region that specifically binds to a target molecule that is different from the target molecule bound by the BTN3A ectodomain.

12. The method according to claim 11, wherein the fusion partner comprises a region that specifically binds an antigen selected from: CTLA-4, Lag-3, BTLA, Tim-3, CD244, CD40, CD40L, CD47, SIRPα, PD-1, and PD-L1.

13. The method according to any of claims 1-12, wherein the BTN3A ectodomain polypeptide comprises a detectable label.

14. The method according to any of claims 1-13, wherein said contacting comprises administering the BTN3A ectodomain polypeptide to an individual with cancer and/or an infectious disease.

15. The method according to claim 14, wherein the BTN3A ectodomain polypeptide is co-administered with an ADCC-inducing antibody.

16. The method according to claim 15, wherein the ADCC-inducing antibody specifically binds to a tumor antigen.

17. The method according to claim 16, wherein the tumor antigen is selected from: CD20, CD52, CD38, HER-2, 17-1A, and EGFR.

18. The method according to any of claims 1-13, wherein said contacting is in vitro or ex vivo.

19. The method according to claim 18, wherein the method comprises contacting the APC or monocyte with a tumor antigen.

20. The method according to claim 19, wherein the method comprises contacting the APC or monocyte with a tumor lystate.

21. The method according to claim 19 or claim 20, wherein the APC or monocyte is contacted with the tumor antigen and/or the tumor lysate in the presence of the BTN3A ectodomain polypeptide.

22. The method according to claim 19 or claim 20, wherein the APC or monocyte is contacted with the tumor antigen and/or tumor lysate prior to or after said contacting with the BTN3A ectodomain polypeptide.

23. The method according to any of claims 18-22, wherein the activated APC is introduced into an individual with cancer and/or an infectious disease.

24. The method according to claim 23, wherein the activated APC is autologous to the individual.

25. The method according to any of claims 1-24, wherein the activated APC is used to cross-prime a naive T cell into an antigen specific effector cell.

26. The method according to claim 25, wherein the activated APC is contacted in vitro or ex vivo with the naive T cell.

27. The method according to claim 25 or claim 26, wherein the antigen specific effector cell is introduced into an individual with cancer and/or an infectious disease.

28. The method according to any of claims 25-27, wherein the naive T cell is autologous to the individual.

29. A pharmaceutical BTN3A ectodomain composition, comprising:
(a) a BTN3A ectodomain polypeptide comprising a BTN3A ectodomain and lacking a BTN3A transmembrane domain; and
(b) a pharmaceutical excipient,
wherein the composition is a unit dose formulation that is effective to activate antigen presenting cells (APCs) in an individual.

30. The composition according to claim 29, wherein the BTN3A ectodomain polypeptide comprises a BTN3A1 ectodomain.

31. The composition according to claim 29, wherein the BTN3A ectodomain polypeptide comprises a BTN3A2 ectodomain.

32. The composition according to claim 29, wherein the BTN3A ectodomain polypeptide comprises a BTN3A3 ectodomain.

33. The composition according to any of claims 29-32, wherein the BTN3A ectodomain comprises an amino acid sequence having 80% or more sequence identity with the amino acid sequence set forth in any of SEQ ID NOs: 10, 13, and 15.

34. The composition according to any of claims 29-33, wherein the BTN3A ectodomain polypeptide comprises a dimerization moiety.

35. The composition according to claim 34, wherein the dimerization moiety comprises an amino acid sequence having 80% or more sequence identity with the amino acid sequence set forth in any of SEQ ID NOs: 31-34.

36. The composition according to any of claims 29-33, wherein the BTN3A ectodomain polypeptide is a monomer.

37. The composition according to any of claims 29-36, wherein the BTN3A ectodomain polypeptide comprises a BTN3A ectodomain and a fusion partner.

38. The composition according to claim 37, wherein the fusion partner is part or whole of an Fc region.

39. The composition according to claim 38, wherein the Fc region is a human IgG4 Fc region.

40. The composition according to claim 39, wherein the BTN3A ectodomain polypeptide comprises an amino acid sequence having 80% or more sequence identity with the amino acid sequence set forth in SEQ ID NO: 30.

41. The composition according to claim 37, wherein the BTN3A ectodomain polypeptide is a multispecific protein and the fusion partner comprises a region that specifically binds to a target molecule that is different from the target molecule bound by the BTN3A ectodomain.

42. The composition according to claim 41, wherein the fusion partner comprises a region that specifically binds to a tumor antigen.

43. The composition according to claim 41, wherein the fusion partner comprises a region that specifically binds an antigen selected from: CTLA-4, Lag-3, BTLA, Tim-3, CD244, CD40, CD40L, CD47, SIRPα, PD-1, and PD-L1.

44. The composition according to any of claims 29-43, wherein the BTN3A ectodomain polypeptide comprises a detectable label.

45. The composition according to any of claims 29-44, further comprising an ADCC-inducing antibody.

46. The composition according to claim 45, wherein the ADCC-inducing antibody specifically binds to a tumor antigen.

47. The composition according to claim 45, wherein the ADCC-inducing antibody specifically binds to an antigen selected from: CD20, CD52, CD38, HER-2, 17-1A, and EGFR.

48. A method of treating an individual having cancer and/or having a chronic infection, the method comprising:
administering to the individual, a pharmaceutical BTN3A ectodomain composition according to any of claims 29-47, in an amount effective to reduce the number of cancer cells and/or infected cells in the individual.

49. The method according to claim 48, wherein the individual is a human.

50. The method according to claim 48 or claim 49, wherein the method comprises co-administering the pharmaceutical BTN3A ectodomain composition with an ADCC-inducing antibody.

51. The method according to claim 50, wherein the ADCC-inducing antibody specifically binds to a tumor antigen.

52. The method according to claim 51, wherein the ADCC-inducing antibody specifically binds to an antigen selected from: CD20, CD52, CD38, HER-2, 17-1A, and EGFR.

53. The method according to any of claims 50-52, wherein the pharmaceutical BTN3A ectodomain composition and the ADCC-inducing antibody are not administered simultaneously.

54. The method according to any of claims 50-52, wherein the pharmaceutical BTN3A ectodomain composition and the ADCC-inducing antibody are administered simultaneously.

55. A BTN3A ectodomain polypeptide, or a nucleic acid encoding said BTN3A ectodomain polypeptide, wherein the BTN3A ectodomain polypeptide comprises a BTN3A ectodomain and a dimerization moiety, and lacks a BTN3A transmembrane domain.

56. The BTN3A ectodomain polypeptide, or nucleic acid encoding said BTN3A ectodomain polypeptide, according to claim 55, wherein the BTN3A ectodomain polypeptide comprises a BTN3A1 ectodomain.

57. The BTN3A ectodomain polypeptide, or nucleic acid encoding said BTN3A ectodomain polypeptide, according to claim 55, wherein the BTN3A ectodomain polypeptide comprises a BTN3A2 ectodomain.

58. The BTN3A ectodomain polypeptide, or nucleic acid encoding said BTN3A ectodomain polypeptide, according to claim 55, wherein the BTN3A ectodomain polypeptide comprises a BTN3A3 ectodomain.

59. The BTN3A ectodomain polypeptide, or nucleic acid encoding said BTN3A ectodomain polypeptide, according to claim 55, wherein the BTN3A ectodomain comprises an amino acid sequence having 80% or more sequence identity with the wild type BTN3A ectodomain amino acid sequence set forth in any of SEQ ID NOs: 10, 13, and 15.

60. A BTN3A ectodomain polypeptide, or a nucleic acid encoding said BTN3A ectodomain polypeptide, wherein the BTN3A ectodomain polypeptide comprises a BTN3A ectodomain and a human IgG4 Fc region, and lacks a BTN3A transmembrane domain.

61. The BTN3A ectodomain polypeptide, or nucleic acid encoding said BTN3A ectodomain polypeptide, according to claim 60, wherein the BTN3A ectodomain polypeptide comprises a BTN3A1 ectodomain.

62. The BTN3A ectodomain polypeptide, or nucleic acid encoding said BTN3A ectodomain polypeptide, according to claim 60, wherein the BTN3A ectodomain polypeptide comprises a BTN3A2 ectodomain.

63. The BTN3A ectodomain polypeptide, or nucleic acid encoding said BTN3A ectodomain polypeptide, according to claim 60, wherein the BTN3A ectodomain polypeptide comprises a BTN3A3 ectodomain.

64. The BTN3A ectodomain polypeptide, or nucleic acid encoding said BTN3A ectodomain polypeptide, according to claim 60, wherein the BTN3A ectodomain comprises an amino acid sequence having 80% or more sequence identity with the wild type BTN3A ectodomain amino acid sequence set forth in any of SEQ ID NOs: 10, 13, and 15.

65. A method for enhancing immune responses to an antigenic compound, comprising:
administering to an individual: (a) a BTN3A ectodomain polypeptide comprising a BTN3A ectodomain and lacking a BTN3A transmembrane domain; and (b) an antigen.

66. The method according to claim 65, wherein the source of the antigen is selected from: a human, a non-human animal, a plant, a bacterial cell, an archaeal cell, a fungus, a virus, a parasite, and a cancer cell.

67. The method according to claim 65 or claim 66, wherein the individual is a mammal.

68. The method according to claim 67, wherein the individual is a human.

69. The method according to any of claims 65-68, wherein the antigen is a vaccine.

70. The method according to claim 69, wherein the vaccine is directed at Tuberculosis, Malaria, Human Immunodeficiency Virus (HIV), RotaVirus, Herpes Simplex Virus (HSV), or Cytomegalovirus (CMV).

71. The method according to claim 70, wherein the vaccine is a cancer vaccine.


SEQUENCE LISTING (43 sequences)

SEQ ID NO: 1 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 2 - protein, 352 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPGEEMLQMRLHFVK

SEQ ID NO: 3 - protein, 379 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPGPPIGQTQQQTRGQGSPVALSQESAQRTDSWGPEEGGESA

SEQ ID NO: 4 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 5 - protein, 311 aa, Homo sapiens
MGIPRAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 6 - protein, 584 aa, Homo sapiens
MKMASSLAFLLLNFHVSLFLVQLLTPCSAQFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREKLQEELKWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 7 - protein, 535 aa, Homo sapiens
MVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQPWIAALAGTLPISLLLLAGASYFLWRQQKEKIALSRETEREREMKEMGYAATEQEISLREWRKIQYMARGEKSLAYHEWKMALFKPADVILDPDTANAILLVSEDQRSVQRAEEPRDLPDNPERFEWRYCVLGCENFTSGRHYWEVEVGDRKEWHIGVCSKNVERKKGWVKMTPENGYWTMGLTDGNKYRALTEPRTNLKLPEPPRKVGIFLDYETGEISFYNATDGSHIYTFPHASFSEPLYPVFRILTLEPTALTICPIPKEVESSPDPDLVPDHSLETPLTPGLANESGEPQAEVTSLLLPAHPGAEVSPSATTNQNHKLQARTEALY

SEQ ID NO: 8 - protein, 5 aa, Homo sapiens
AAAAA

SEQ ID NO: 9 - protein, 5 aa, Homo sapiens
AAAAA

SEQ ID NO: 10 - protein, 217 aa, Artificial sequence
  Synthetic sequence
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQ

SEQ ID NO: 11 - protein, 217 aa, Artificial sequence; identical to SEQ ID NO: 10
  Synthetic sequence

SEQ ID NO: 12 - protein, 217 aa, Artificial sequence; identical to SEQ ID NO: 10
  Synthetic sequence

SEQ ID NO: 13 - protein, 217 aa, Artificial sequence
  Synthetic sequence
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQ

SEQ ID NO: 14 - protein, 217 aa, Artificial sequence; identical to SEQ ID NO: 13
  Synthetic sequence

SEQ ID NO: 15 - protein, 217 aa, Artificial sequence
  Synthetic sequence
QFSVLGPSGPILAMVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQ

SEQ ID NO: 16 - protein, 204 aa, Artificial sequence
  Synthetic sequence
MVGEDADLPCHLFPTMSAETMELRWVSSSLRQVVNVYADGKEVEDRQSAPYRGRTSILRDGITAGKAALRIHNVTASDSGKYLCYFQDGDFYEKALVELKVAALGSDLHIEVKGYEDGGIHLECRSTGWYPQPQIKWSDTKGENIPAVEAPVVADGVGLYAVAASVIMRGSSGGGVSCIIRNSLLGLEKTASISIADPFFRSAQ

SEQ ID NO: 17 - protein, 5 aa, Artificial sequence
  Synthetic sequence
AAAAA

SEQ ID NO: 18 - protein, 5 aa, Artificial sequence
  Synthetic sequence
AAAAA

SEQ ID NO: 19 - protein, 5 aa, Artificial sequence
  Synthetic sequence
AAAAA

SEQ ID NO: 20 - protein, 464 aa, Artificial sequence
  Synthetic sequence
MYRMQLLSCIALSLALVTNSQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQAAAPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 21 - protein, 303 aa, Artificial sequence
  Synthetic sequence
MLLVNQSHQGFNKEHTSKMVSAIVLYVLLAAAAHSAFAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQAAARMKQLEDKVEELLSKNYHLENEVARLKKLVGAASGADHHHHHHHH

SEQ ID NO: 22 - protein, 5 aa, Artificial sequence
  Synthetic sequence
AAAAA

SEQ ID NO: 23 - protein, 5 aa, Artificial sequence
  Synthetic sequence
AAAAA

SEQ ID NO: 24 - protein, 5 aa, Artificial sequence
  Synthetic sequence
AAAAA

SEQ ID NO: 25 - protein, 8 aa, Artificial sequence
  Synthetic sequence
HHHHHHHH

SEQ ID NO: 26 - protein, 38 aa, Artificial sequence
  Synthetic sequence
MLLVNQSHQGFNKEHTSKMVSAIVLYVLLAAAAHSAFA

SEQ ID NO: 27 - protein, 20 aa, Artificial sequence
  Synthetic sequence
MYRMQLLSCIALSLALVTNS

SEQ ID NO: 28 - protein, 5 aa, Artificial sequence
  Synthetic sequence
AAAAA

SEQ ID NO: 29 - protein, 5 aa, Artificial sequence
  Synthetic sequence
AAAAA

SEQ ID NO: 30 - protein, 224 aa, Artificial sequence
  Synthetic sequence
PPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 31 - protein, 37 aa, Artificial sequence
  Synthetic sequence
RMKQLEDKVEELLSKNYHLENEVARLKKLVGAASGAD

SEQ ID NO: 32 - protein, 47 aa, Artificial sequence
  Synthetic sequence
NDLDAYEREAEKLEKKNEVLRNRLAALENELATLRQEVASMKQELQS

SEQ ID NO: 33 - protein, 42 aa, Artificial sequence
  Synthetic sequence
NEKEELKSKKAELRNRIEQLKQKREQLKQKIANLRKEIEAYK

SEQ ID NO: 34 - protein, 41 aa, Artificial sequence
  Synthetic sequence
SIAATLENDLARLENENARLEKDIANLERDLAKLEREEAYF

SEQ ID NO: 35 - protein, 5 aa, Artificial sequence
  Synthetic sequence
GSGGS

SEQ ID NO: 36 - protein, 6 aa, Artificial sequence
  Synthetic sequence
GGSGGS

SEQ ID NO: 37 - protein, 4 aa, Artificial sequence
  Synthetic sequence
GGGS

SEQ ID NO: 38 - protein, 4 aa, Artificial sequence
  Synthetic sequence
GGSG

SEQ ID NO: 39 - protein, 5 aa, Artificial sequence
  Synthetic sequence
GGSGG

SEQ ID NO: 40 - protein, 5 aa, Artificial sequence
  Synthetic sequence
GSGSG

SEQ ID NO: 41 - protein, 5 aa, Artificial sequence
  Synthetic sequence
GSGGG

SEQ ID NO: 42 - protein, 5 aa, Artificial sequence
  Synthetic sequence
GGGSG

SEQ ID NO: 43 - protein, 5 aa, Artificial sequence
  Synthetic sequence
GSSSG